(19) United States
(12) Patent Application Publication
(10) Pub. No.: US 2024/0382541 A1
(43) Pub. Date: Nov. 21, 2024

(54) FORMULATIONS TO STABILIZE VIRUS-BASED THERAPEUTICS

(71) Applicant: Boehringer Ingelheim International GmbH, Ingelheim am Rhein (DE)

(72) Inventors: Martin DASS, Biberach-Bergerhausen (DE); Ludger Markus ICKENSTEIN, Biberach an der Riss (DE); Thomas KRIEHUBER, Maselheim (DE); Katharina REICHE, Biberach an der Riss (DE)

(21) Appl. No.: 18/665,666

(22) Filed: May 16, 2024

(30) Foreign Application Priority Data
May 17, 2023 (EP) 23173808.9

(51) Int. Cl. A61K 35/766; A61K 47/10; A61K 47/18; A61K 47/26; A61K 47/42

(52) U.S. Cl. CPC A61K 35/766; A61K 47/10; A61K 47/18; A61K 47/183; A61K 47/26; A61K 47/42

(57) ABSTRACT

The present invention relates to viral formulations for administration to a subject.

### SEQUENCE DISCLOSURE

This application includes, as part of its disclosure, a “Sequence Listing XML” pursuant to 37 C.F.R. § 1.831 (a) which is submitted in XML file format via the USPTO patent electronic filing system in a file named “01-3567-US-1_SL.xml” created on Jun. 11, 2024, and having a size of 49,152 bytes, which is hereby incorporated by reference herein in its entirety.

### FIELD OF THE INVENTION

The present invention relates to a pharmaceutical formulation for virus-based therapeutics and virus-based cancer vaccines. More specifically, the present invention relates to a pharmaceutical formulation for oncolytic viruses as disclosed herein.

### BACKGROUND OF THE INVENTION

Oncolytic viruses, such as the vesicular stomatitis virus (VSV) containing the glycoprotein (GP) of the lymphocytic chorio-meningitis virus (VSV-GP), are an emerging class of biologicals, which selectively replicate in and kill cancer cells. The oncolytic viruses described herein can spread within tumors and efficiently induce tumor cell lysis resulting in cell death. Furthermore, additional genes may be cloned into the virus genome and expression of said proteins can stimulate and/or direct the immune system against tumor cells. In addition, oncolytic viruses expressing cancer-specific antigens can be co-administered together with the antigen and thus enhance and prolong its immune-stimulating effect. Accordingly, oncolytic viruses are beneficial for the treatment and/or the prevention of cancer.

The development of a formulation that stabilizes oncolytic viruses upon storage is challenging. Isolated concentrated live viruses are typically unstable during long term storage and sensitive towards elevated temperatures, mechanical stress, freeze-thaw cycles and the like. To be effective as a therapeutic agent, live viruses need to be formulated to preserve the activity and to prevent aggregation and thus, the formation of visible and subvisible particles (SvPs). Owing to their structural complexity, viruses and in particular VSV-GP tends to self-associate and aggregate. The colloidal stability of virus particles must be ensured, and aggregation must be reduced below compendial limits by formulation development efforts. The number of freezing and thawing events, for instance for filling and labelling of vials, as well as the freezing and storage conditions, such as the freezing and thawing rate and the storage temperature impact greatly the infectivity and colloidal stability especially when viruses are not stored as a frozen liquid at temperatures below the glass transition temperature of the maximally freeze-concentrated solution (T_{g}′).

Although several formulations for virus-based therapeutics are described in the literature for specific viruses, it is commonly understood that each virus species, genus, or family needs to be formulated in its own specific formulation.

Accordingly, there is a need for stable liquid, frozen liquid, and dry formulations (such as lyophilized formulations) for virus-based therapeutics and in particular formulations for oncolytic viruses, such as VSV, in particular VSV-GP, which are suitable for parenteral administration, including intravenous or intratumoral injection into human patients. There exists furthermore a need for formulations that exhibit e.g., increased colloidal stability and no or only minor loss of the biological activity of the therapeutic virus during extended periods of storage at various temperatures.

### SUMMARY OF THE INVENTION

The present invention addresses the needs mentioned above by providing stable formulations and in particular stable liquid and frozen liquid formulations for virus-based therapeutics.

In a first aspect the invention relates to a pharmaceutical composition comprising an enveloped virus, AND a protein agent and/or a poly(ethylene oxide)/poly(propylene oxide) block copolymer.

In one embodiment relating to the first aspect, the poly(ethylene oxide) and poly(propylene oxide) block copolymer is a poloxamer. In one embodiment relating to the first aspect, the poly(ethylene oxide) and poly(propylene oxide) block copolymer is poloxamer 188.

In one embodiment relating to the first aspect, the protein agent is albumin or gelatin. In one embodiment relating to the first aspect, the protein agent is human serum albumin or recombinant human albumin.

In one embodiment relating to the first aspect, the poly(ethylene oxide) and poly(propylene oxide) block copolymer is poloxamer 188, and the protein agent is human serum albumin or recombinant human albumin. In a related embodiment, the pharmaceutical composition comprises poloxamer 188 in a concentration between 0.01-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.01-40 g/L, 0.01-30 g/L, 0.01-20 g/L, 0.01-10 g/L, 0.01-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L. In a further related embodiment, the pharmaceutical composition comprises human serum albumin or recombinant human albumin in a concentration between 0.05-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.05-40 g/L, 0.05-30 g/L, 0.05-20 g/L, 0.05-10 g/L, 0.05-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

In one embodiment relating to the first aspect and any of its embodiments, the pharmaceutical composition further comprises at least one of an amino acid, a buffer, or a sugar. In a related embodiment, the amino acid is selected from the group consisting of alanine, arginine, phenylalanine, glutamic acid, glycine, methionine, lysine, and glutamine, preferably arginine. In a related embodiment, the buffer is selected from the group consisting of acetate, citrate, histidine, succinate, HEPES, tartrate, phosphate, citrate/phosphate, lactate, and Tris, preferably Tris. In a related embodiment, the buffer has a concentration between 1-100 mM, 1-90 mM, 1-80 mM, 1-70 mM, 1-60 mM, 1-50 mM, 1-40 mM, 1-30 mM, 1-20 mM, or 1-10 mM. In a related embodiment, the sugar is selected from the group consisting of dextrose, fructose, galactose, glucose, raffinose, trehalose, and sucrose, preferably trehalose. In a related embodiment, the sugar has a concentration between 10-1000 mM, 10-900 mM, 10-800 mM, 10-700 mM, 10-600 mM, 10-500 mM, 10-400 mM, 10-300 mM, 10-200 mM, 20-1000 mM, 20-900 mM, 20-800 mM, 20-700 mM, 20-600 mM, 20-500 mM, 20-400 mM, 20-300 mM, 20-200 mM, 30-1000 mM, 30-900 mM, 30-800 mM, 30-700 mM, 30-600 mM, 30-500 mM, 30-400 mM, 30-300 mM, 30-200 mM, 40-1000 mM, 40-900 mM, 40-800 mM, 40-700 mM, 40-600 mM, 40-500 mM, 40-400 mM, 40-300 mM, 40-200 mM, 50-1000 mM, 50-900 mM, 50-800 mM, 50-700 mM, 50-600 mM, 50-500 mM, 50-400 mM, 50-300 mM, or 50-200 mM.

In one embodiment relating to the first aspect and any of its embodiments, the composition further comprises one or more sugar alcohol. In a related embodiment, the one or more sugar alcohol is selected from the group consisting of: mannitol, sorbitol, xylitol, maltitol, maltitol symp, lactitol, inositol, glycerol erythritol, isomalt, and hydrogenated starch hydroxylate. In a further related embodiment, the one or more sugar alcohol is mannitol and/or sorbitol, preferably a combination of mannitol and sorbitol.

In one embodiment relating to the first aspect, the composition is substantially free of chloride, preferably substantially free of NaCl.

In one embodiment relating to the first aspect, the pH of the composition is between 5 to 9, or between 6 to 9, or between 6.5 to 8.5, or between 6.5 to 8.0, preferably between 7.0 and 8.0. In a related embodiment, the pH of the composition is adjusted with phosphoric acid or sodium phosphate.

In one embodiment relating to the first aspect, the pharmaceutical composition comprises:
an enveloped virus,
at least one of a buffer, an amino acid, or a sugar,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a buffer, AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a sugar, AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a buffer and a sugar, AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition
an enveloped virus,
a buffer and a sugar, AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
at least one of a buffer, an amino acid, or a sugar, wherein the buffer is selected from the group consisting of acetate, citrate, histidine, succinate, HEPES, tartrate, phosphate, citrate/phosphate, lactate, and Tris, preferably Tris,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a buffer, wherein the buffer is selected from the group consisting of acetate, citrate, histidine, succinate, HEPES, tartrate, phosphate, citrate/phosphate, lactate, and Tris, preferably Tris,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a buffer and a sugar, wherein the buffer is selected from the group consisting of acetate, citrate, histidine, succinate, HEPES, tartrate, phosphate, citrate/phosphate, lactate, and Tris, preferably Tris,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
a buffer and a sugar, wherein the buffer is selected from the group consisting of acetate, citrate, histidine, succinate, HEPES, tartrate, phosphate, citrate/phosphate, lactate, and Tris, preferably Tris,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
at least one of a buffer, an amino acid, or a sugar, wherein the sugar is selected from the group consisting of dextrose, fructose, galactose, glucose, raffinose, trehalose, and sucrose, preferably trehalose,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a buffer, wherein the amino acid is selected from the group consisting of alanine, arginine, phenylalanine, glutamic acid, glycine, methionine, lysine, and glutamine, preferably arginine,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a sugar, wherein the sugar is selected from the group consisting of dextrose, fructose, galactose, glucose, raffinose, trehalose, and sucrose, preferably trehalose,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a buffer and a sugar, wherein the sugar is selected from the group consisting of dextrose, fructose, galactose, glucose, raffinose, trehalose, and sucrose, preferably trehalose,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
a buffer and a sugar, wherein the sugar is selected from the group consisting of dextrose, fructose, galactose, glucose, raffinose, trehalose, and sucrose, preferably trehalose,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition
an enveloped virus,
at least one of a buffer, an amino acid, or a sugar, wherein the amino acid is selected from the group consisting of alanine, arginine, phenylalanine, glutamic acid, glycine, methionine, lysine, and glutamine, preferably arginine,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a sugar, wherein the amino acid is selected from the group consisting of alanine, arginine, phenylalanine, glutamic acid, glycine, methionine, lysine, and glutamine, preferably arginine,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a buffer and a sugar, wherein the amino acid is selected from the group consisting of alanine, arginine, phenylalanine, glutamic acid, glycine, methionine, lysine, and glutamine, preferably arginine,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
at least one of a buffer, an amino acid or a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably arginine;
wherein the sugar is selected from the group consisting of dextrose, fructose, galactose, glucose, raffinose, trehalose, and sucrose, preferably trehalose,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a buffer, wherein the buffer is selected from the group consisting of acetate, citrate, histidine, succinate, HEPES, tartrate, phosphate, citrate/phosphate, lactate, and Tris, preferably Tris; wherein the amino acid is selected from the group consisting of alanine, arginine, phenylalanine, glutamic acid, glycine, methionine, lysine, and glutamine, preferably arginine,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid and a sugar, wherein the amino acid is selected from the group consisting of alanine, arginine, phenylalanine, glutamic acid, glycine, methionine, lysine, and glutamine, preferably arginine; wherein the sugar is selected from the group consisting of dextrose, fructose, galactose, glucose, raffinose, trehalose, and sucrose, preferably trehalose,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition
an enveloped virus,
an amino acid and a buffer and a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably arginine; wherein the sugar is selected from the group consisting of dextrose, fructose, galactose, glucose, raffinose, trehalose, and sucrose, preferably trehalose,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
a buffer and a sugar, wherein the buffer is selected from the group consisting of acetate, citrate, histidine, succinate, HEPES, tartrate, phosphate, citrate/phosphate, lactate, and Tris, preferably Tris; wherein the amino acid is selected from the group consisting of alanine, arginine, phenylalanine, glutamic acid, glycine, methionine, lysine, and glutamine, preferably arginine,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
a buffer, wherein the buffer is Tris
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition
an enveloped virus,
an amino acid, wherein the amino acid is arginine,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
a sugar, wherein the sugar is trehalose or sucrose,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
an amino acid, wherein the amino acid is arginine,
a sugar, wherein the sugar is trehalose or sucrose,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
a buffer, wherein the buffer is Tris
a sugar, wherein the sugar is trehalose or sucrose,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
a buffer, wherein the buffer is Tris
an amino acid, wherein the amino acid is arginine,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect, the pharmaceutical composition comprises:
an enveloped virus,
a buffer, wherein the buffer is Tris
an amino acid, wherein the amino acid is arginine,
a sugar, wherein the sugar is trehalose or sucrose,
AND
a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

In one embodiment relating to the first aspect and any of its embodiments comprising a buffer, the buffer has a concentration between 1-100 mM, 1-90 mM, 1-80 mM, 1-70 mM, 1-60 mM, 1-50 mM, 1-40 mM, 1-30 mM, 1-20 mM, or 1-10 mM.

In one embodiment relating to the first aspect and any of its embodiments comprising a sugar, the sugar has a concentration between 10-1000 mM, 10-900 mM, 10-800 mM, 10-700 mM, 10-600 mM, 10-500 mM, 10-400 mM, 10-300 mM, 10-200 mM, 20-1000 mM, 20-900 mM, 20-800 mM, 20-700 mM, 20-600 mM, 20-500 mM, 20-400 mM, 20-300 mM, 20-200 mM, 30-1000 mM, 30-900 mM, 30-800 mM, 30-700 mM, 30-600 mM, 30-500 mM, 30-400 mM, 30-300 mM, 30-200 mM, 40-1000 mM, 40-900 mM, 40-800 mM, 40-700 mM, 40-600 mM, 40-500 mM, 40-400 mM, 40-300 mM, 40-200 mM, 50-1000 mM, 50-900 mM, 50-800 mM, 50-700 mM, 50-600 mM, 50-500 mM, 50-400 mM, 50-300 mM, or 50-200 mM.

In one embodiment relating to the first aspect and any of its embodiments, the pharmaceutical composition comprises poloxamer 188 in a concentration between 0.01-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.01-40 g/L, 0.01-30 g/L, 0.01-20 g/L, 0.01-10 g/L, 0.01-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

In one embodiment relating to the first aspect and any of its embodiments, the pharmaceutical composition comprises human serum albumin or recombinant human albumin in a concentration between 0.05-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.05-40 g/L, 0.05-30 g/L, 0.05-20 g/L, 0.05-10 g/L, 0.05-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

In one embodiment relating to the first aspect, the pharmaceutical composition comprises:
about 1-100 mM Tris,
about 10-500 mM Arginine,
about 10-500 mM Trehalose
about 0.1-5 mg/ml Poloxamer 188,
about 0.5-10 mg/ml recombinant Human albumin, and
a pH of about 6 to 8.

In one embodiment relating to the first aspect, the pharmaceutical composition comprises:
an enveloped virus
about 10 mM Tris
about 150 mM Arginine
about 100 mM Trehalose
about 0.5 mg/ml Poloxamer 188,
about 2 mg/ml recombinant Human albumin, and
a pH of about 7.5.

In one embodiment relating to the first aspect and any of its embodiments, the pH of the composition is adjusted with phosphoric acid or sodium phosphate.

In one embodiment relating to the first aspect and any of its embodiments, the enveloped virus is a rhabdoviridae, preferably a vesiculovirus or vesicular stomatitis virus (VSV). In a related embodiment, the enveloped virus is a recombinant vesicular stomatitis virus (VSV), wherein the gene coding for the glycoprotein G of the vesicular stomatitis virus is replaced by the gene coding for the glycoprotein GP of LCMV, and/or the glycoprotein G is replaced by the glycoprotein GP of LCMV. In a related embodiment, the pharmaceutical composition comprises the enveloped virus, preferably the vesiculovirus or vesicular stomatitis virus (VSV), in a concentration of at least 1×10⁵ TCID₅₀/mL, at least 1×10⁶ TCID₅₀/mL, at least 1×10⁷ TCID₅₀/mL, at least 1×10⁸ TCID₅₀/mL, at least 1×10⁹ TCID₅₀/mL, or at least 1×10⁹ TCID₅₀/mL. In a further related embodiment, the pharmaceutical composition comprises the enveloped virus, preferably the vesiculovirus or vesicular stomatitis virus (VSV), in a concentration range between 1×10⁵ TCID₅₀/mL to 1×10¹² TCID₅₀/mL, between 1×10⁶ TCID₅₀/mL to 1×10¹² TCID₅₀/mL, between 1×10⁷ TCID₅₀/mL to 1×10¹² TCID₅₀/mL, between 1×10⁸ TCID₅₀/mL to 1×10¹² TCID₅₀/mL, 1×10⁵ TCID₅₀/mL to 1×10¹¹ TCID₅₀/mL, 1×10⁵ TCID₅₀/mL to 1×10¹⁰ TCID₅₀/mL, or 1×10⁵ TCID₅₀/mL to 1×10⁹ TCID₅₀/mL.

In one embodiment relating to the first aspect and any of its embodiments, the pharmaceutical composition is a liquid or frozen liquid pharmaceutical composition. In a related embodiment, the composition is frozen and stored at a temperature of about −80° C., −70° C., −60° C., −50° C., −40° C., −35° C., −30° C., −25° C., −20° C., −15° C., −10° C., or −5° C. In a related embodiment, the invention relates to a product produced by lyophilizing the liquid pharmaceutical composition. A further embodiment relates to a pharmaceutical composition comprising water and the product.

In one embodiment relating to the first aspect and any of its embodiments, the invention further relates to a dry pharmaceutical composition produced by a method comprising removing water from a pharmaceutical composition according to the first aspects and any of its embodiments. In a related embodiment, the dry pharmaceutical composition is frozen to obtain a pharmaceutical composition comprising ice prior to removing water. In a related embodiment, the method further comprises placing the liquid pharmaceutical composition in a vacuum under controlled temperatures and pressure to remove the water. In a further related embodiment, the method is lyophilization. In a further related embodiment, the dry pharmaceutical composition comprises less than about (0.5%-5%) w/w water. A further embodiment relates to a pharmaceutical composition comprising water and the dry pharmaceutical composition.

### BRIEF DESCRIPTION OF THE FIGURES

FIG. 1 Geometric mean values of VSV-GP-Cargo1 infectivity following storage of liquid formulations at a temperature of 5° C. Error bars indicate the standard deviation (n=2 for control, n=3 for others, measured in 3 replicas, each). A frozen crude harvest sample serves as a control.

FIG. 2 Geometric mean values of VSV-GP-Cargo1 infectivity following storage of liquid formulations at a temperature of 25° C. Error bars indicate the standard deviation (n=2 for control, n=3 for others, measured in 3 replicas, each). A frozen crude harvest sample serves as a control.

FIG. 3 Geometric mean values of SvPs≥10 μm of VSV-GP-Cargo1 following storage of liquid formulations at a temperature of 5° C. Error bars indicate the standard deviation (n=2 for control, n=3 for others, measured in 3 replicas, each).

FIG. 4 Geometric mean values of SvPs≥10 μm of VSV-GP-Cargo1 following storage of liquid formulations at a temperature of 25° C. Error bars indicate the standard deviation (n=2 for control, n=3 for others, measured in 3 replicas, each).

FIG. 5 Geometric mean values of VSV-GP-Cargo1 infectivity following no, one, or three freeze/thaw cycles and storage of liquid formulations at a temperature of 25° C. Error bars indicate the standard deviation (n=2 for control, n=3 for others, measured in 3 replicas, each). A frozen crude harvest sample serves as a control.

FIG. 6 Geometric mean values of SvPs≥10 μm of VSV-GP-Cargo1 following no, one, or three freeze/thaw cycles of liquid formulations. Error bars indicate the standard deviation (n=2 for control, n=3 for others, measured in 3 replicas, each).

FIG. 7 Geometric mean values of VSV-GP-Cargo1 infectivity following 1, 3, or 5 freeze/thaw cycles at temperatures of −20° C. and +25° C. of frozen liquid formulations. Error bars indicate the standard deviation (n=2 for control, n=3 for others, measured in 3 replicas, each). A frozen crude harvest sample serves as a control.

FIG. 8 Geometric mean values of VSV-GP-Cargo1 infectivity following 1, 3, or 5 freeze/thaw cycles at temperatures of −80° C. and +25° C. of frozen liquid formulations. Error bars indicate the standard deviation (n=2 for control, n=3 for others, measured in 3 replicas, each). A frozen crude harvest sample serves as a control.

FIG. 9 Geometric mean values of SvPs≥10 μm of VSV-GP-Cargo1 following 1, 3, or 5 freeze/thaw cycles at temperatures of −20° C. and +25° C. of frozen liquid formulations. Error bars indicate the standard deviation (n=2 for control, n=3 for others, measured in 3 replicas, each).

FIG. 10 Geometric mean values of SvPs≥10 μm of VSV-GP-Cargo1 following 1, 3, or 5 freeze/thaw cycles at temperatures of −80° C. and +25° C. of frozen liquid formulations. Error bars indicate the standard deviation (n=2 for control, n=3 for others, measured in 3 replicas, each).

FIG. 11 Geometric mean values of VSV-GP-Cargo1 infectivity following storage of lyophilized formulations at a temperature of 25° C. Error bars indicate the standard deviation (n=2 for control, n=3 for others, measured in 3 replicas, each). As a control serves a frozen crude harvest sample. The first time point represents the measurement before lyophilization. The second value (0 weeks) is the value immediately after lyophilisation and reconstitution.

FIG. 12 Geometric mean values of SvPs≥10 μm of VSV-GP-Cargo1 following storage of lyophilized formulations at a temperature of 25° C. Error bars indicate the standard deviation (n=2 for control, n=3 for others, measured in 3 replicas, each). The first time point represents the measurement before lyophilization. The second value is the value immediately after lyophilisation and reconstitution.

FIG. 13 Geometric mean values of SvPs≥2 μm of VSV-GP following storage of frozen liquid formulations for one (T1w) or two weeks (T2w) at a temperature of 25° C. or after one freeze/thaw cycle (T-FT) (n=1 measured in 3 replicas, each). Raw data were multiplied by a dilution factor of 100. The initial to value is the pre-frozen value.

FIG. 14 Geometric mean values of SvPs≥10 μm of VSV-GP following storage of frozen liquid formulations for one (T1w) or two weeks (T2w) at a temperature of 25° C. or after one freeze/thaw cycle (T-FT) (n=1 measured in 3 replicas, each). Raw data were multiplied by a dilution factor of 100. The initial to value is the pre-frozen value.

FIG. 15 Geometric mean values of VSV-GP infectivity following storage of frozen liquid formulations for one (T1w) or two weeks (T2w) at a temperature of 25° C. or after one freeze/thaw cycle (T-FT) (n=1). The initial to value is the pre-frozen value.

FIG. 16 Overview of infectious titers of VSV-GP DP samples at different time points after freeze-thaw (T-FT) or storing for 1 week (T-1w) or 2 weeks (T-2w) at 25° C., determined by TCID₅₀ analysis (n=3). The starting material is VSV-GP in elution buffer.

FIG. 17 Geometric mean values of SvPs≥2 μm of VSV-GP following storage of frozen liquid formulations for one week (T1w) or two weeks (T2w) at a temperature of 25° C. or after one freeze/thaw cycle (T-FT) (n=1). Raw data were multiplied by a dilution factor of 20. The initial to value is the pre-frozen value.

FIG. 18 Geometric mean values of SvPs≥10 μm of VSV-GP DP following storage of frozen liquid formulations for one week (T1w) or two weeks (T2w) at a temperature of 25° C. or after one freeze/thaw cycle (T-FT) (n=1). Raw data were multiplied by a dilution factor of 20. The initial to value is the pre-frozen value.

FIG. 19 Geometric mean values of VSV-GP infectivity following storage of frozen liquid formulations for one week (T1w) or two weeks (T2w) at a temperature of 25° C. or after one freeze/thaw cycle (T-FT). Error bars indicate the standard deviation (n=4 measured in 3 replicas, each). The initial to value is the pre-frozen value. A VSV-GP batch with a titer of 1-2×10⁹ TCID₅₀/mL serves as a control.

FIG. 20 Geometric mean values of VSV-GP infectivity following freeze-thaw (T-FT) or storage for two weeks at a temperature of 25° C. (T-2w 25° C.). Error bars indicate the standard deviation (n=4 measured in 3 replicas, each).

### DETAILED DESCRIPTION OF THE INVENTION

In the following detailed description, numerous specific details are set forth to provide a full understanding of the present invention. It will be apparent, however, to a person ordinarily skilled in the art that the subject technology may be practiced without some of these specific details. In other instances, well-known structures and techniques have not been shown in detail so as not to obscure the present invention. The headings are included merely for convenience to assist in reading and shall not be understood to limit the invention to specific aspects or embodiments.

In one aspect, a formulation of the present invention is useful to stabilize the infectious titer of an enveloped virus. In another aspect, the formulation helps to preserve the infectious titer and/or the colloidal stability of an enveloped virus after one or more freeze-thaw cycles. In a related aspect, the formulation is useful to preserve the infectious titer and/or colloidal stability of an enveloped virus upon storage at elevated temperatures, preferably at a temperature of 2-8° C., at room temperature, or even at temperatures above room temperature. In a related aspect, the formulation is useful to preserve titer and/or activity of an enveloped virus for storage of the virus at various temperatures for certain periods of time.

In another aspect, a formulation of the present invention may reduce, and/or slow down the formation of visible and/or subvisible particles (SvPs) in formulations containing the enveloped virus. In a related aspect, the formulation containing the enveloped virus has a reduced amount of visible and/or SvPs.

In yet another aspect, a formulation of the present invention helps to preserve the infectious titer of an enveloped virus. In a related aspect, the infectious titer is retained or the loss in infectious titer over time is slowed down. In a related aspect, the loss in infectious titer due to prolonged storage, repeated freeze-thaw cycles, storage at elevated temperatures or mechanical stress is slowed down.

In another aspect, a formulation of the present invention helps to suppress, slow down, or prevent aggregation of an enveloped virus. In another aspect, the formulation is useful to suppress, reduce, or prevent clouding of a formulation containing the enveloped virus.

In another aspect, a formulation of the present invention helps to suppress, slow down, or prevent the formation of visible and/or SvPs within a formulation containing an enveloped virus. Preferably, the formulation containing the enveloped virus is substantially free of visible and/or SvPs.

In another aspect, a formulation of the present invention containing an enveloped virus preserves the infectivity of the enveloped virus and/or slows down the loss of infectivity upon storage at a temperature of 25° C.

In another aspect, the formulation is useful to preserve the infectious titer and/or the colloidal stability of an enveloped virus upon storage at a temperature of 2-8° C.

In another aspect, a formulation of the present invention containing an enveloped virus has visible/sub-visible particle concentrations that are below the compendial limits.

In an aspect relating to any of the aforementioned aspects, the formulation containing the enveloped virus is a liquid formulation, a frozen liquid formulation, or a dry formulation such as a lyophilized formulation.

In another aspect, a liquid and/or frozen liquid formulation of the present invention containing an enveloped virus preserves infectivity of the enveloped virus and/or slows down loss of infectivity upon storage at a temperature of 25° C. for at least 28 days.

In another aspect, a liquid and/or frozen liquid formulation of the present invention containing an enveloped virus is useful to reduce visible and/or SvPs formation upon storage at a temperature of 25° C. for up to 28 days.

In another aspect, a liquid and/or frozen liquid formulation of the present invention containing an enveloped virus largely prevents the formation of particles≥2 μm and/or ≥10 μm upon freeze-thaw stress.

In another aspect, a liquid and/or frozen liquid formulation of the present invention containing an enveloped virus largely prevents the formation of particles≥2 μm and/or ≥10 μm following shaking stress.

In another aspect, a liquid and/or frozen liquid formulation of the present invention containing an enveloped virus is useful to reduce, slow down, and/or prevent formation of visible particles upon storage at a temperature of 25° C. In a related aspect, a liquid and/or frozen liquid formulation of the present invention containing an enveloped virus the concentration of SvPs≥2 μm upon storage at a temperature of 25° C. and after one freeze-thaw cycle is below 5000 particles/mL, below 4000 particles/mL 3000 particles/mL, below 2000 particles/mL, or below 1000 particles/mL. In a further related aspect, a liquid and/or frozen liquid formulation of the present invention containing an enveloped virus the concentration of SvPs≥10 μm upon storage at a temperature of 25° C. and after one freeze-thaw cycle is below 6000 particles/container, below 5000 particles/container, below 4000 particles/container, below 3000 particles/container, below 2000 particles/container, below 1000 particles/container, below 800 particles/container, below 600 particles/container, below 400 particles/container, below 200 particles/container, or below 100 particles/container.

In another aspect, adjustment of formulation buffer with trisodium phosphate (Na₃PO₄) and/or phosphoric acid (H₃PO₄) together with omission of sodium chloride reduces osmolality and increases T_{g} value.

In another aspect, adjustment of formulation buffer with phosphoric acid (H₃PO₄) instead of HCl reduces osmolality in formulations without trehalose and increases T_{g} value by 13° C. to 19° C.

In another aspect, omission of sodium chloride eliminates formation of chloride salts upon freezing.

In relation to any of the aforementioned aspects, the titer of a particular formulation can be tested by determining the TCID₅₀ according to the method as shown in the Examples. In relation to any of the aforementioned aspects, the particles can be assessed according to the method as described in the Examples.

Without being bound by theory, it has been found that poloxamer and particular poloxamer 188 reduces the formation of aggregates and/or particles in liquid and/or frozen liquid oncolytic virus formulations, in particular following freeze/thaw cycling.

It was also found that recombinant human albumin (rHA) was able to reduce the formation of aggregates and/or particles in frozen liquid formulations, presumably by interaction with the virus surface. Moreover, rHA was found to stabilize the infectivity of lyophilized virus upon storage in frozen liquid formulations.

Surprisingly, it was also found that in liquid and/or frozen liquid formulations stress induced particles in rHA containing formulations could be effectively prevented by the addition of poloxamer 188.

The pharmaceutical formulations and compositions described herein are particularly useful for the formulation of enveloped viruses. According to the present invention, the term “enveloped virus” refers to any of the genera of enveloped viruses capable of infecting humans, such as but not limited to herpesvirus, poxvirus, orthomyxovirus, paramyxoviruses, rhabdovirus, and filoviruses. As used herein a rhabdovirus can belong to the genus of: almendravirus, curiovirus, cytorhabdovirus, dichorhavirus, ephemerovirus, hapavirus, ledantevirus, lyssavirus, novirhabdovirus, nucleorhabdovirus, perhabdovirus, sigmavirus, sprivivirus, sripuvirus, tibrovirus, tupavirus, varicosavirus, or vesiculovirus.

Preferably, the enveloped virus is replication competent. Further preferred are oncolytic viruses and replication competent oncolytic viruses. In this respect, the term oncolytic is meant in its regular meaning, which is known in the art and refers to the ability of a virus to infect and lyse (break down) cancer cells but not normal cells (to any significant extend). Preferably, the oncolytic virus is capable of replication within cancer cells. Oncolytic activity may be tested in different assay systems known to the skilled artisan (an exemplary in vitro assay is described by Muik et al., Cancer Res., 74(13), 3567-78, 2014). It is understood that an oncolytic virus may infect and lyse only specific types of cancer cells. Also, the oncolytic effect may vary depending on the type of cancer cells. It is understood that an oncolytic virus is a live virus that is able to infect and replicate in cancer cells.

The enveloped virus of any of the embodiments can be a recombinant or non-recombinant enveloped virus, preferably a recombinant enveloped virus, preferably a recombinant enveloped virus belonging to the family of rhabdoviridae, more preferably a recombinant vesiculovirus, and even more preferably a recombinant vesicular stomatitis virus.

The term “recombinant” refers to a virus, more particularly an enveloped virus, comprising an exogenous nucleic acid sequence inserted in its genome, which is not naturally present in the parent virus. A recombinant virus thus refers to a nucleic acid or virus made by an artificial combination of two or more segments of nucleic acid sequence of synthetic or semisynthetic origin which does not occur in nature or is linked to another nucleic acid in an arrangement not found in nature. The artificial combination is most commonly accomplished by artificial manipulation of isolated segments of nucleic acids, using well-established genetic engineering techniques. Generally, a “recombinant” enveloped virus as described herein refers to enveloped viruses that are produced by standard genetic engineering methods, e.g., enveloped viruses of the present invention are thus genetically engineered or genetically modified enveloped virus. The term “recombinant enveloped virus” thus includes enveloped viruses, which have stably integrated recombinant nucleic acid in their genome.

Characterizing features of members of the family of rhabdoviruses include a negative-sense, single-stranded RNA of 10.8-16.1 kb, which are mostly unsegmented and a genome encoding for at least 5 genes encoding the structural proteins nucleoprotein (N), large protein (L), phosphoprotein (P), matrix protein (M), and glycoprotein (G).

Vesiculovirus species have been defined primarily by serological means coupled with phylogenetic analysis of the genomes. Biological characteristics such as host range and mechanisms of transmission are also used to distinguish viral species within the genus. As such, the genus of vesiculovirus form a distinct monophyletic group well-supported by maximum likelihood trees inferred from complete L sequences.

Viruses assigned to different species within the genus vesiculovirus may have one or more of the following characteristics: A) a minimum amino acid sequence divergence of 20% in L; B) a minimum amino acid sequence divergence of 10% in N; C) a minimum amino acid sequence divergence of 15% in G; D) can be distinguished in serological tests; and E) occupy different ecological niches as evidenced by differences in hosts and or arthropod vectors.

In a preferred embodiment the vesicular stomatitis virus encodes in its genome at least for a vesicular stomatitis virus nucleoprotein (N), large protein (L), phosphoprotein (P), matrix protein (M), glycoprotein (G).

In a preferred embodiment the vesicular stomatitis virus encodes in its genome at least for a vesicular stomatitis virus nucleoprotein (N) comprising an amino acid sequence as set forth in SEQ ID NO:1 or a functional variant at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:1, a phosphoprotein (P) comprising an amino acid sequence as set forth in SEQ ID NO:2 or a functional variant at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:2, a large protein (L) comprising an amino acid sequence as set forth in SEQ ID NO:3 or a functional variant at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:3, and a matrix protein (M) comprising an amino acid sequence as set forth in SEQ ID NO:4 or a functional variant at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:4.

It is understood by the skilled artisan that modifications to the vesicular stomatitis virus nucleoprotein (N), large protein (L), phosphoprotein (P), matrix protein (M), or glycoprotein (G) sequence can be made without losing the basic functions of those proteins. Such functional variants as used herein retain all or part of their basic function or activity. The protein L for example is the polymerase and has an essential function during transcription and replication of the virus. A functional variant thereof must retain at least part of this ability. A good indication for retention of basic functionality or activity is the successful production of viruses, including these functional variants, that are still capable to replicate and infect tumor cells. Production of viruses and testing for infection and replication in tumor cells may be tested in different assay systems known to the skilled artisan (an exemplary in vitro assay is described by Muik et al., Cancer Res., 74(13), 3567-78, 2014).

In a preferred embodiment the vesicular stomatitis virus encodes in its genome at least for a vesicular stomatitis virus nucleoprotein (N), large protein (L), phosphoprotein (P), matrix protein (M) and glycoprotein (G), wherein the large protein (L) comprises an amino acid sequence having a sequence identity≥80% of SEQ ID NO:3.

In a preferred embodiment the vesicular stomatitis virus encodes in its genome at least for a vesicular stomatitis virus nucleoprotein (N), large protein (L), phosphoprotein (P), matrix protein (M) and glycoprotein (G), wherein the nucleoprotein (N) comprises an amino acid sequence having a sequence identity≥90% of SEQ ID NO:1.

In a further preferred embodiment, the vesicular stomatitis virus encodes in its genome at least for a vesicular stomatitis virus nucleoprotein (N), large protein (L), phosphoprotein (P), matrix protein (M) and glycoprotein (G), wherein the large protein (L) comprises an amino acid sequence having a sequence identity≥80% of SEQ ID NO:3 and the nucleoprotein (N) comprises an amino acid sequence having a sequence identity≥90% of SEQ ID NO:1.

It is known that certain wildtype VSV strains are considered to be neurotoxic. It is also reported that infected individuals are able to rapidly mount a strong humoral response with high antibody titers directed mainly against the glycoprotein. Neutralizing antibodies targeting the glycoprotein G of VSV are able to limit virus spread and thereby mediate protection of individuals from virus re-infection. Virus neutralization, however, limits repeated application of the virus to the cancer patient.

To eliminate these drawbacks the wildtype glycoprotein G may be replaced with the glycoprotein from another virus. In this respect replacing the glycoprotein refers to (i) replacement of the gene coding for the wild-type glycoprotein G with the gene coding for the glycoprotein GP of another virus, and/or (ii) replacement of the wild-type glycoprotein G with the glycoprotein GP of another virus.

In a preferred embodiment the enveloped virus is a vesicular stomatitis virus and the VSV glycoprotein G is replaced with the glycoprotein GP of the lymphocytic choriomeningitis virus (LCMV), preferably with the glycoprotein of the strain WE-HPI. Such VSV is for example described in WO2010/040526 and named VSV-GP.

Hence, in a most preferred embodiment the enveloped virus is a recombinant vesicular stomatitis virus, wherein the gene coding for the glycoprotein G of the vesicular stomatitis virus is replaced by the gene coding for the glycoprotein GP of LCMV, and/or the glycoprotein G is replaced by the glycoprotein GP of LCMV.

In a preferred embodiment, the gene coding for the glycoprotein GP of the LCMV encodes for a protein with an amino acid sequence as shown in SEQ ID NO:5 or an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO:5 while the functional properties of the enveloped virus comprising a glycoprotein GP encoding an amino acid sequence as shown in SEQ ID NO:5 are maintained.

It is to be understood that a recombinant enveloped virus may encode in its genome further cargos, such as tumor antigens, further chemokines, cytokines, or other immunomodulatory elements.

In a preferred embodiment the RNA genome of the vesicular stomatitis virus comprises or consists of a sequence as shown in SEQ ID NO: 6 or 7 or 8. Furthermore, the RNA genome of the vesicular stomatitis virus may also consist of or comprise those sequences, wherein nucleic acids of the RNA genome are exchanged according to the degeneration of the genetic code, without leading to an alteration of respective amino acid sequence. In a further preferred embodiment, the RNA genome of the vesicular stomatitis virus comprises or consists of a coding sequence identical or at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6 or 7 or 8.

### Definitions

As used herein, the terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions×100). In some embodiments, the two sequences that are compared are the same length after gaps are introduced within the sequences, as appropriate (e.g., excluding additional sequence extending beyond the sequences being compared).

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

The term “about” shall generally mean an acceptable degree of error or variation for the quantity measured given the nature or precision of the measurements. Typical, exemplary degrees of error or variation are within 5% or within 3% or within 1% of a given value or range of values. For example, the expression of “about 100” includes 105 and 95 or 103 and 97 or 101 and 99, and all values in between (e.g., 95.1, 95.2, etc. for range of 95-105; or 97.1, 97.2, etc. for the range of 97-103; 99.1, 99.2, etc. for the range of 99-101). Numerical quantities given herein are approximates unless stated otherwise, meaning that the term “about” can be inferred when not expressly stated.

The general embodiments “comprising” or “comprise” as used herein encompass the more specific embodiment “consisting of”. Furthermore, singular and plural forms are not used in a limiting way. As used herein, the singular forms “a”, “an” and “the” designate both the singular and the plural, unless expressly stated to designate the singular only.

A “pharmaceutical formulation” or “formulation” refers to the process but also the product of a process in which an active drug or agent is combined with chemical substances to produce a final medicinal or drug product, the final formulation therefore refers to medicinal products such as liquids, frozen liquids, dry preparations, or compositions. Therefore, in one embodiment, a pharmaceutical formulation is a pharmaceutical composition.

A “pharmaceutical composition” refers in this context to a liquid, frozen liquid or dry preparation which is in such form as to permit the biological activity of the active ingredient(s) to be unequivocally effective, and which contains no additional components which are significantly toxic to the subjects to which the composition would be administered. Such compositions are sterile.

A “dry preparation” or a “dry pharmaceutical composition” is prepared by removing the liquid of a preparation containing the enveloped virus that has been formulated in a liquid solution. The removal of the liquid can be accomplished by e.g., evaporation, such as by the application of the liquid solution to a solid substrate and evaporation of the liquid and/or by sublimation such as by lyophilization (freeze-drying). The dry preparations/dry pharmaceutical compositions of the present invention are stored as dried formulations generally with 0.5% to 10.0% (w/w) residual moisture content (RMC). The dry preparations/dry pharmaceutical compositions can be reconstituted prior to administration, e.g., in an aqueous solutions, such as but not limited to sterile water, saline solutions, aqueous dextrose or glycerol and the like. In particular embodiments the dry preparations/dry pharmaceutical compositions of the present invention are stored as dried formulations comprising 0.5% to 5% (w/w) residual moisture content. In more particular embodiments the dry preparations/dry pharmaceutical compositions of the present invention are stored as dried formulations comprising 0.5% to 3% (w/w) residual moisture content.

It will be understood that a dry pharmaceutical composition according to the invention will be originally prepared as a liquid pharmaceutical composition. Therefore, it will be further understood that if concentration ranges and/or pH ranges are given for a dry pharmaceutical composition, said concentration ranges and/or pH ranges refer to the originally prepared liquid pharmaceutical composition before it is dried and/or to the liquid pharmaceutical composition obtained after reconstitution of the dry pharmaceutical composition with an aqueous solution, e.g. with water.

It follows that in one embodiment a pharmaceutical composition according to the invention is a liquid pharmaceutical composition comprising an aqueous solution, preferably water, and any of the (dry) pharmaceutical compositions as described herein.

It further follows that in another embodiment, a pharmaceutical composition according to the invention is a liquid pharmaceutical composition obtained by reconstituting a dry pharmaceutical composition according to the invention in an aqueous solution, preferably water.

It will be understood that the different formulations described here can be provided as liquids, frozen liquids, and/or dry formulations. Hence, if not specified otherwise, the general term formulation or pharmaceutical formulation or composition or pharmaceutical composition encompasses all of liquids, frozen liquids, and/or dry compositions/formulations.

As used herein, the term “water” refers to water for injection.

The “pharmaceutically acceptable” excipients (vehicles, additives) are those, which are suitable for parenteral administration to a subject.

In one embodiment, the pharmaceutical formulation of the present invention is stable.

“Stability” refers to chemical stability and physical stability and can be evaluated qualitatively and/or quantitatively using various analytical techniques that are described in the art and are reviewed in for example in: Moving oncolytic viruses into the clinic: clinical-grade production, purification, and characterization of diverse oncolytic viruses. Mol Ther Methods Clin Dev. 2016 Apr. 6; 3:16018. doi: 10.1038/mtm.2016.18. PMID: 27088104; PMCID: PMC4822647. Those methods include the evaluation of aggregate and particle formation (for example using high-performance size exclusion chromatography (HP-SEC), by measuring turbidity, sub-visible particles by light obscuration (LO) of or microflow imaging (MFI=flow imaging microscopy (FIM)), dynamic imaging analysis (DIA), and/or by visual inspection of color and clarity); by assessing charge heterogeneity using cation exchange chromatography (CEX), or capillary isoelectric focusing; mass spectrometric analysis; capillary gel electrophoresis (CGE) analysis; peptide map (for example tryptic or Lys-C digest) analysis; and evaluating biological activity (infectivity); etc. In order to measure stability, a sample of the formulation of the invention may be tested in a stability study, wherein a sample is exposed for a selected time period to a stress condition followed by quantitative and qualitative analysis of the chemical and physical stability and infectivity using an adequate analytical technique.

Accordingly, stability can be measured at a selected temperature for a selected time period for instance by storing a sample at different temperatures such as −80° C., −20°, 2-8° C., or 25° C. for up to 12 months and by using for instance HP-SEC, CEX, MFI, LO, CGE, or infectivity for qualitative and quantitative analysis.

According to the above, a “stable formulation” is one in which the formulation containing the enveloped virus is physically and chemically stable and/or retains its biological activity upon storage.

“Physical stability” refers substantially in context of the invention to an enveloped virus having little or no signs of aggregation, precipitation, and/or loss of infectivity. Methods to access the physical stability are for example SEC, LO, MFI, or dynamic imaging analysis (DIA) and visual inspections. For SEC, extensive peak broadening or tailing might be considered as a significantly difference in the context of the invention under the tested conditions depending on the column used, operating pressure, flow rate of the buffer. Using MFI, a significant increase in the number of particles, in particular particles larger than 2 μm and/or 10 μm might be considered as a significantly difference especially if particle numbers exceed compendial limits. Methods to access the infectivity are the median tissue culture infective dose, i.e., the quantity of a cytopathogenic agent, which will produce a cytopathic effect in 50% of the seeded cells.

The term “stress” or “stress condition” in context of the invention refers to e.g., mechanical stress, thermal stress, light stress, or stress resulting from freezing and thawing and particularly as shown in the Example section. Methods and conditions to simulate mechanical stress, thermal stress, light stress, or stress resulting from freezing and thawing are diverse and known to those skilled in the art. Mechanical stress may be for example shaking with 300 rpm at room temperature for up to 48 hours or vigilantly shaking the virus-containing vial by hand. Thermal stress refers for example to the storage at decreased or increased temperatures for an amount of time, in one example samples may be stored at a temperature of 5° C., 25° C., or 30° C., wherein for instance 25° C. and 30° C. refer to an accelerated stress condition. Light stress might be for example storing the samples at a light intensity of about 1100 lux for 5 days at various temperatures. Samples might be exposed to stress from freezing and thawing by exposing the samples to several cycles of freezing, e.g., at a temperature of −80° C. for 24 hours and thawing at room temperature for 2 hours, wherein the cycles are repeated 3-5 times.

The term “substantially free of chloride” has the meaning that no source of chloride ions has been added to the pharmaceutical composition, preferably no source of chloride ions has been exogenously added to the pharmaceutical composition. More preferably, the pharmaceutical composition is free of chloride ions. Buffers

As used herein “buffer” refers to a buffered solution that resists changes in pH by the action of its acid-base conjugate components. The “pH” herein refers to the acidity or basicity of the composition at room temperature. Standard methods to measure the pH of a composition are known to the skilled in the art. Typically, measuring pH consists of calibrating the instrument, placing the electrodes in a well-mixed sample, and then reading the pH directly from the pH meter.

In various aspects, the pharmaceutical composition may comprise a buffer. The exemplary buffers of the present invention include acetate, citrate, histidine, succinate, HEPES, tartrate, phosphate, citrate/phosphate, lactate, and Tris.

### Sugars

In various aspects, the pharmaceutical composition may comprise a sugar or a combination of several sugars. The exemplary sugars of the present invention include dextrose, fructose, galactose, glucose, raffinose, trehalose, or sucrose.

### Sugar Alcohols

In various aspects, the pharmaceutical composition may comprise a sugar alcohol or a combination of several sugar alcohols. The exemplary sugar alcohols of the present invention include mannitol, sorbitol, xylitol, maltitol, maltitol symp, lactitol, inositol, glycerol erythritol, isomalt, or hydrogenated starch hydroxylate.

### Poloxamer

As used herein, “EO-PO block copolymer” means a copolymer consisting of blocks of poly(ethylene oxide) and poly(propylene oxide).

As used herein, “Pluronic” means EO-PO block copolymers in the EOₓ-PO_{y}-EO_{z} configuration. This configuration is also referred to as “poloxamer”.

For the generic term poloxamer, these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g., P188=poloxamer with a polyoxypropylene molecular mass of 7680-9510 g/mol and a 20% polyoxyethylene content). For poloxamer 188 the PPO chain contains a unit number ranging from 25 to 30, and each PEO block is composed of 75 to 85 EO units in average (L. Bollenbach, J. Buske, K. Mader, P. Garidel, International Journal of Pharmaceutics, Volume 620, 2022).

As used herein a “protein agent” is albumin, gelatin, serum albumin, recombinant albumin, bovine serum albumin, porcine serum albumin, human serum albumin, recombinant human albumin, preferably human serum albumin (HSA) or recombinant human albumin (rHA).

In some embodiments, the pharmaceutical composition comprises human serum albumin (HSA), preferably, recombinant human albumin (rHA). HSA is the most abundant protein found in human blood plasma. As used herein the term “recombinant” in the context of “HA” means that the rHA is a genetically engineered product or made by recombinant production methods. A rHA is not derived from (isolated or purified from) a natural product (e.g., human plasma) but may be produced e.g. via genetically engineered cells to produce the rHA—although other methods to obtain rHA may be used equally by the skilled artisan.

All of the following tables should be read in the way that the formulation comprises or consists of the components in the indicated concentration ranges. Optional components may or may not be part of the formulation. If the terms “at least one” or “one or more” are used in conjunction with a single concentration range then said concentration range is to be understood to apply individually for each component, e.g. if two amino acids are present and only one concentration range from 1 to 300 mM is given, then both amino acid individually have a concentration range from 1 to 300 mM.

TABLE 1
Typical concentration ranges of the components of the formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| (optionally) A buffer | 1 to 100 mM |
| (optionally) At least one sugar | 1 to 500 mM |
| One or more amino acids | each in 1 to 300 mM |
| (optionally) One or more sugar alcohols | 1 to 200 mM |
| A poly(ethylene oxide) and poly(propylene | 0.01 g/L to 50 g/L |
| oxide) block copolymer, preferably | 0.1 g/L to 50 g/L |
| poloxamer 188 and/or | |
| a protein agent, preferably human serum | |
| albumin or recombinant human albumin | |

TABLE 1a
Typical concentration ranges of the components of the formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| (optionally) A Buffer, selected from the group | 1 to 100 mM |
| consisting of: Acetate, citrate, histidine, succinate, | |
| HEPES, tartrate, phosphate, citrate/phosphate, | |
| lactate, or Tris | |
| (optionally) At least one sugar, selected from the | 1 to 500 mM |
| group consisting of: | |
| Dextrose, fructose, galactose, glucose, raffinose, | |
| trehalose, and sucrose | |
| One or more amino acids, selected from the group | 1 to 300 mM |
| consisting of: | |
| Arginine, alanine, phenylalanine, glycine, | |
| glutamine, glutamic acid, methionine, and lysine | |
| (optionally) One or more sugar alcohols, selected | 1 to 200 mM |
| from the group consisting of: | |
| Mannitol, sorbitol, xylitol, maltitol, maltitol | |
| symp, lactitol, inositol, glycerol erythritol, | |
| isomalt, and hydrogenated starch hydroxylate | |
| A poly(ethylene oxide) and poly(propylene oxide) | 0.01 g/L to 50 g/L |
| block copolymer, preferably poloxamer 188 | 0.1 g/L to 50 g/L |
| and/or | |
| a protein agent: albumin, gelatin, preferably | |
| human serum albumin or recombinant human | |
| albumin | |

In the following formulations according to the invention are shown. Preferably, said formulations are provided as liquid or frozen liquid formulations.

TABLE 2
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| A Buffer | 1 to 100 mM |
| (Optionally) A sugar | 1 to 500 mM |
| An amino acid | 1 to 300 mM |
| A poly(ethylene oxide) and poly(propylene oxide) | 0.01 g/L to 50 g/L |
| block copolymer, preferably poloxamer 188 | |
| and/or | |
| Protein agent: human serum albumin, preferably | 0.1 g/L to 50 g/L |
| recombinant human albumin | |

TABLE 2a
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| A Buffer selected from the group consisting of: | 1 to 100 mM |
| acetate, citrate, histidine, succinate, HEPES, | |
| tartrate, phosphate, citrate/phosphate, lactate, | |
| and Tris; preferably Tris | |
| (Optionally) A sugar | 1 to 500 mM |
| An amino acid | 1 to 300 mM |
| A poly(ethylene oxide) and poly(propylene oxide) | 0.01 g/L to 50 g/L |
| block copolymer, preferably poloxamer 188 | |
| and/or | |
| Protein agent: human serum albumin, preferably | 0.1 g/L to 50 g/L |
| recombinant human albumin | |

TABLE 2b
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| A Buffer | 1 to 100 mM |
| (Optionally) A sugar | 1 to 500 mM |
| An amino acid, selected from the group consisting | 1 to 300 mM |
| of: arginine, alanine, phenylalanine, glycine, | |
| glutamine, glutamic acid, methionine, and | |
| lysine; preferably arginine | |
| A poly(ethylene oxide) and poly(propylene oxide) | 0.01 g/L to 50 g/L |
| block copolymer, preferably poloxamer 188 | |
| and/or | |
| Protein agent: human serum albumin, preferably | 0.1 g/L to 50 g/L |
| recombinant human albumin | |

TABLE 2c
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| A Buffer, selected from the group consisting | 1 to 100 mM |
| of: acetate, citrate, histidine, succinate, | |
| HEPES, tartrate, phosphate, citrate/ | |
| phosphate, lactate, and Tris | |
| (Optionally) A sugar, selected from the group | 1 to 500 mM |
| consisting of: dextrose, fructose, galactose, | |
| glucose, raffinose, trehalose, and sucrose | |
| An Amino acid, selected from the group | 1 to 300 mM |
| consisting of: arginine, alanine, phenylalanine, | |
| glycine, glutamine, glutamic acid, methionine, | |
| and lysine | |
| A poly(ethylene oxide) and poly(propylene oxide) | 0.01 g/L to 50 g/L |
| block copolymer, preferably poloxamer 188 | |
| and/or | |
| Protein agent: human serum albumin, preferably | 0.1 g/L to 50 g/L |
| recombinant human albumin | |

TABLE 2d
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Tris buffer | 1 to 100 mM |
| (Optionally) A sugar, selected from the group | 1 to 500 mM |
| consisting of: dextrose, fructose, galactose, | |
| glucose, raffinose, trehalose, and sucrose | |
| An Amino acid selected from the group | 1 to 300 mM |
| consisting of: arginine, alanine, phenylalanine, | |
| glycine, glutamine, glutamic acid, methionine, | |
| and lysine | |
| A poly(ethylene oxide) and poly(propylene oxide) | 0.01 g/L to 50 g/L |
| block copolymer, preferably poloxamer 188 | |
| and/or | |
| Protein agent: human serum albumin, preferably | 0.1 g/L to 50 g/L |
| recombinant human albumin | |

TABLE 2e
Typical concentration ranges of the components of a formulation,
preferably a liquid or frozen liquid formulation

| | Concentration |
|---|---|
| Component | Range |
| An enveloped virus, preferably a vesiculovirus or | 1 × 10⁵ TCID₅₀/mL to |
| a vesicular stomatitis virus, more preferably | 1 × 10¹² TCID₅₀/mL |
| VSV-GP | |
| A Tris buffer | 1 to 100 mM |
| (Optionally) A sugar, selected from the group | 1 to 500 mM |
| consisting of: dextrose, fructose, galactose, | |
| glucose, raffinose, trehalose, and sucrose | |
| Arginine as an amino acid | 1 to 300 mM |
| A poly(ethylene oxide) and poly(propylene oxide) | 0.01 g/L to 50 g/L |
| block copolymer, preferably poloxamer 188 | |
| and/or | |
| Protein agent: human serum albumin, preferably | 0.1 g/L to 50 g/L |
| recombinant human albumin | |

TABLE 2f
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Tris buffer | 1 to 100 mM |
| (Optionally) A sugar, selected from the group | 1 to 500 mM |
| consisting of: dextrose, fructose, galactose, | |
| glucose, raffinose, trehalose, and sucrose | |
| Arginine as an amino acid | 1 to 300 mM |
| poloxamer 188 | 0.01 g/L to 50 g/L |
| and/or | |
| recombinant human albumin | 0.1 g/L to 50 g/L |

TABLE 2g
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Tris buffer | 1 to 100 mM |
| (Optionally) A sugar, selected from the group | 1 to 500 mM |
| consisting of: dextrose, fructose, galactose, | |
| glucose, raffinose, trehalose, and sucrose | |
| Arginine as an amino acid | 1 to 300 mM |
| poloxamer 188 | 0.01 g/L to 50 g/L |
| and | |
| recombinant human albumin | 0.1 g/L to 50 g/L |

TABLE 2h
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Tris buffer | 1 to 100 mM |
| Trehalose as a sugar | 1 to 500 mM |
| Arginine as an amino acid | 1 to 300 mM |
| poloxamer 188 | 0.01 g/L to 50 g/L |
| and | |
| recombinant human albumin | 0.1 g/L to 50 g/L |

TABLE 2i
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Buffer, selected from the group consisting of: | 1 to 100 mM |
| acetate, citrate, histidine, succinate, HEPES, | |
| tartrate, phosphate, citrate/phosphate, lactate, | |
| and Tris | |
| (Optionally) A sugar, selected from the group | 1 to 500 mM |
| consisting of: dextrose, fructose, galactose, | |
| glucose, raffinose, trehalose, and sucrose | |
| An Amino acid selected from the group consisting | 1 to 300 mM |
| of: arginine, alanine, phenylalanine, glycine, | |
| glutamine, glutamic acid, methionine, or lysine | |
| poloxamer 188 | 0.01 g/L to 50 g/L |
| and/or | |
| recombinant human albumin | 0.1 g/L to 50 g/L |

TABLE 2j
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Buffer, selected from the group consisting of: | 1 to 100 mM |
| acetate, citrate, histidine, succinate, HEPES, | |
| tartrate, phosphate, citrate/phosphate, lactate, | |
| and Tris | |
| (Optionally) A sugar, selected from the group | 1 to 500 mM |
| consisting of: dextrose, fructose, galactose, | |
| glucose, raffinose, trehalose, and sucrose | |
| An Amino acid selected from the group consisting | 1 to 300 mM |
| of: arginine, alanine, phenylalanine, glycine, | |
| glglutamine, utamic acid, methionine, and lysine | |
| poloxamer 188 | 0.01 g/L to 50 g/L |
| and | |
| recombinant human albumin | 0.1 g/L to 50 g/L |

TABLE 2k
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Buffer, selected from the group consisting of: | 1 to 100 mM |
| acetate, citrate, histidine, succinate, HEPES, | |
| tartrate, phosphate, citrate/phosphate, lactate, | |
| and Tris | |
| (Optionally) A sugar, selected from the group | 1 to 500 mM |
| consisting of: dextrose, fructose, galactose, | |
| glucose, raffinose, trehalose, and sucrose | |
| Arginine as an amino acid | 1 to 300 mM |
| poloxamer 188 | 0.01 g/L to 50 g/L |
| and | |
| recombinant human albumin | 0.1 g/L to 50 g/L |

TABLE 2l
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Tris buffer | 5 to 50 mM |
| Trehalose as a sugar | 50 to 250 mM |
| Arginine as an amino acid | 50 to 250 mM |
| poloxamer 188 | 2.5 g/L to 10 g/L |
| and | |
| recombinant human albumin | 2.5 g/L to 10 g/L |

In a preferred embodiment, a liquid or frozen liquid formulation comprises an enveloped virus, about 10 mM Tris, about 150 mM arginine, about 106 mM trehalose, about 5 mg/ml rHA, a pH between 7.0 and 8.0, preferably about pH 7.5.

In a preferred embodiment, a liquid or frozen liquid formulation comprises an enveloped virus, about 9.75 mM Tris, about 146 mM arginine, about 103 mM trehalose, about 5 mg/mL rHA, a pH between 7.0 and 8.0, preferably about pH 7.5.

In a preferred embodiment, prior to spiking with rHA and poloxamer 188 the liquid or frozen liquid formulation comprises an enveloped virus, about 10 mM Tris, about 150 mM arginine, about 200 mM trehalose, a pH between 7.0 and 8.0, preferably about pH 7.5. After spiking rHA and poloxamer 188 to a final concentration of about 5 mg/mL each, the liquid or frozen liquid formulation comprises the following composition: an enveloped virus, about 9.75 mM Tris, about 146 mM arginine, about 195 mM trehalose, about 5 mg/mL rHA, about 5 mg/mL poloxamer 188, a pH between 7.0 and 8.0, preferably about pH 7.5.

In any of the above-mentioned formulations, preferably liquid or frozen liquid formulation embodiments the buffer concentration can be in any concentration range between 1 to 100 mM, such as 1 to 90 mM, 1 to 80 mM, 1 to 70 mM, 1 to 60 mM, 1 to 50 mM. Preferably, the buffer concentration is between 5 mM and 50 mM, 5 mM and 40 mM, 5 mM and 30 mM, or 5 and 20 mM. Preferably, the formulation, preferably liquid or frozen liquid formulation comprises a Tris buffer and more preferably a Tris buffer in a concentration between 5 and 50 mM.

In any of the above mentioned formulations, preferably liquid or frozen liquid formulation embodiments the sugar concentration can be in any concentration range between 1 to 500 mM, such as 1 to 490 mM, 1 to 480 mM, 1 to 470 mM, 1 to 460 mM, 1 to 450 mM, 1 to 440 mM, 1 to 430 mM, 1 to 420 mM, 1 to 410 mM, 1 to 400 mM, 1 to 390 mM, 1 to 380 mM, 1 to 370 mM, 1 to 360 mM, 1 to 350 mM, 1 to 340 mM, 1 to 330 mM, 1 to 320 mM, 1 to 310 mM, 1 to 300 mM, 1 to 290 mM, 1 to 280 mM, 1 to 270 mM, 1 to 260 mM, 1 to 250 mM, 1 to 240 mM, 1 to 230 mM, 1 to 220 mM, 1 to 210 mM, 1 to 200 mM. Preferably, the sugar concentration is between 50 mM and 300 mM, 50 mM and 250 mM, 50 mM and 200 mM. Preferably, the formulation, preferably liquid or frozen liquid formulation comprises a sugar, such as dextrose, fructose, galactose, glucose, raffinose, trehalose, or sucrose. More preferably the sugar is trehalose and in a concentration between 50 and 250 mM.

In any of the above mentioned formulations, preferably liquid or frozen liquid formulation embodiments the amino acid concentration can be in any concentration range between 1 to 300 mM such as 1 to 290 mM, 1 to 280 mM, 1 to 270 mM, 1 to 260 mM, 1 to 250 mM, 1 to 240 mM, 1 to 230 mM, 1 to 220 mM, 1 to 210 mM, 1 to 200 mM, 1 to 190 mM, 1 to 180 mM, 1 to 170 mM, 1 to 160 mM, 1 to 150 mM, 1 to 140 mM, 1 to 130 mM, 1 to 120 mM, 1 to 110 mM, 1 to 100 mM, 1 to 90 mM, 1 to 80 mM, 1 to 70 mM, 1 to 60 mM, 1 to 50 mM, 1 to 40 mM, 1 to 30 mM, 1 to 20 mM, 1 to 10 mM. Preferably, the amino acid concentration is between 50 mM and 300 mM, 50 mM and 290 mM, 50 mM or 280 mM, 50 mM and 270 mM, 50 mM and 260 mM, 50 mM and 250 mM, 50 mM and 240 mM, 50 mM and 230 mM, 50 mM and 220 mM, 50 mM and 210 mM, 50 mM and 200 mM, 50 mM and 190 mM, 50 mM and 180 mM, 50 mM and 170 mM, 50 mM and 160 mM, 50 mM and 150 mM, 50 mM and 140 mM, 50 mM and 130 mM, 50 mM and 120 mM, 50 mM and 110 mM, 50 mM and 100 mM. Preferably, the formulations, preferably liquid or frozen liquid formulation comprises an amino acid, such as arginine, alanine, phenylalanine, glycine, glutamine, glutamic acid, methionine, or lysine. More preferably the amino acid is arginine. Most preferred is arginine in a concentration between 50 and 300 mM or 50 and 250 mM or 50 and 200 mM.

In any of the above mentioned formulations, preferably liquid or frozen liquid formulation embodiments the poloxamer concentration can be in any concentration range between 0.01 g/L to 50 g/L, such as 0.1 g/L to 50 g/L, 1 g/L to 50 g/L, 1.1 g/L to 50 g/L, 1.2 g/L to 50 g/L, 1.3 g/L to 50 g/L, 1.4 g/L to 50 g/L, 1.5 g/L to 50 g/L, 1.6 g/L to 50 g/L, 1.7 g/L to 50 g/L, 1.8 g/L to 50 g/L, 1.9 g/L to 50 g/L, 2.0 g/L to 50 g/L, 2.1 g/L to 50 g/L, 2.2 g/L to 50 g/L, 2.3 g/L to 50 g/L, 2.4 g/L to 50 g/L, 2.5 g/L to 50 g/L. More preferred ranges include 1 g/L to 45 g/L, 1 g/L to 40 g/L, 1 g/L to 35 g/L, 1 g/L to 30 g/L, 1 g/L to 25 g/L, 1 g/L to 20 g/L, 1 g/L to 15 g/L, 1.5 g/L to 10 g/L, 2.0 g/L to 10 g/L, or 2.5 g/L to 10 g/L. Preferably, the formulations, preferably liquid or frozen liquid formulation comprises a pharmaceutically acceptable poloxamer. More preferably the poloxamer is poloxamer 188. Most preferred is poloxamer 188 in a concentration between 1 g/L to 20 g/L, 1 g/L to 15 g/L, 1.5 g/L to 10 g/L, 2.0 g/L to 10 g/L, or 2.5 g/L to 10 g/L.

In any of the above mentioned formulations, preferably liquid or frozen liquid formulation embodiments the protein agent concentration can be in any concentration range between 0.1 g/L to 50 g/L, such as 1 g/L to 50 g/L, 1.1 g/L to 50 g/L, 1.2 g/L to 50 g/L, 1.3 g/L to 50 g/L, 1.4 g/L to 50 g/L, 1.5 g/L to 50 g/L, 1.6 g/L to 50 g/L, 1.7 g/L to 50 g/L, 1.8 g/L to 50 g/L, 1.9 g/L to 50 g/L, 2.0 g/L to 50 g/L, 2.1 g/L to 50 g/L, 2.2 g/L to 50 g/L, 2.3 g/L to 50 g/L, 2.4 g/L to 50 g/L, 2.5 g/L to 50 g/L. More preferred ranges include 1 g/L to 45 g/L, 1 g/L to 40 g/L, 1 g/L to 35 g/L, 1 g/L to 30 g/L, 1 g/L to 25 g/L, 1 g/L to 20 g/L, 1 g/L to 15 g/L, 1.5 g/L to 10 g/L, 2.0 g/L to 10 g/L, or 2.5 g/L to 10 g/L. Preferably, the formulations, preferably liquid or frozen liquid formulation comprises recombinant human albumin. Most preferred is recombinant human albumin in a concentration between 1 g/L to 20 g/L, 1 g/L to 15 g/L, 1.5 g/L to 10 g/L, 2.0 g/L to 10 g/L, or 2.5 g/L to 10 g/L.

The pH of the above-mentioned formulations, preferably liquid or frozen liquid formulation embodiments are usually kept in a range between 5 to 9, or between 6 to 9, or between 6.5 to 8.5, or between 6.5 to 8.0, preferably between 7.0 and 8.0.

In any of the above-mentioned formulations, preferably liquid or frozen liquid formulation embodiments the enveloped virus is preferably a vesiculovirus, more preferably a vesicular stomatitis virus and most preferred a vesicular stomatitis virus having the glycoprotein G replaced with the glycoprotein GP of the lymphocytic choriomeningitis virus (LCMV). In any of the above-mentioned formulations, preferably liquid or frozen liquid formulation embodiments the virus concentration can be in any concentration range between 1×10⁵ TCID₅₀/mL to 1×10¹² TCID₅₀/mL or in a concentration of at least 1×10⁵ TCID₅₀/mL, at least 1×10⁶ TCID₅₀/mL, at least 1×10⁷ TCID₅₀/mL, at least 1×10⁸ TCID₅₀/mL, or at least 1×10⁹ TCID₅₀/mL. Other ranges include between 1×10⁶ TCID₅₀/mL to 1×10¹² TCID₅₀/mL, between 1×10⁶ TCID₅₀/mL to 1×10¹¹ TCID₅₀/mL and the like.

In the following further formulations according to the invention are shown.

TABLE 3
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| (optionally) At least one of a buffer, a sugar, | Buffer 1 to 100 mM |
| or an amino acid | Sugar 1 to 500 mM |
| | Amino acid 1 to 300 mM |
| As poly(ethylene oxide) and poly(propylene | 0.01 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3a
Typical concentration ranges of the components of a formulation,
preferably a liquid or frozen liquid formulation

| | Concentration |
|---|---|
| Component | Range |
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Buffer selected from the group consisting of: | 1 to 100 mM |
| acetate, citrate, histidine, succinate, HEPES, | |
| tartrate, phosphate, citrate/phosphate, lactate, | |
| and Tris; preferably Tris | |
| As poly(ethylene oxide) and poly(propylene | 0.01 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3b
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more | 1 × 10¹² TCID₅₀/mL |
| preferably VSV-GP | |
| A sugar, selected from the group consisting of: | 1 to 500 mM |
| dextrose, fructose, galactose, glucose, | |
| raffinose, trehalose, and sucrose | |
| As poly(ethylene oxide) and poly(propylene | 0.01 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3c
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| An Amino acid selected from the group | 1 to 300 mM |
| consisting of: arginine, alanine, phenylalanine, | |
| glycine, glutamine, glutamic acid, methionine, | |
| and lysine | |
| As poly(ethylene oxide) and poly(propylene | 0.01 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3d
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Buffer selected from the group consisting of: | 1 to 100 mM |
| acetate, citrate, histidine, succinate, HEPES, | |
| tartrate, phosphate, citrate/phosphate, lactate, | |
| and Tris; preferably Tris | |
| A sugar selected from the group consisting of: | 1 to 500 mM |
| dextrose, fructose, galactose, glucose, | |
| raffinose, trehalose, and sucrose | |
| As poly(ethylene oxide) and poly(propylene | 0.01 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3d
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Buffer selected from the group consisting of: | 1 to 100 mM |
| acetate, citrate, histidine, succinate, HEPES, | |
| tartrate, phosphate, citrate/phosphate, lactate, | |
| and Tris; preferably Tris | |
| An Amino acid selected from the group | 1 to 300 mM |
| consisting of: arginine, alanine, phenylalanine, | |
| glycine, glutamine, glutamic acid, methionine, | |
| and lysine | |
| As poly(ethylene oxide) and poly(propylene | 0.01 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3e
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Buffer selected from the group consisting of: | 1 to 100 mM |
| acetate, citrate, histidine, succinate, HEPES, | |
| tartrate, phosphate, citrate/phosphate, lactate, | |
| and Tris; preferably Tris | |
| An Amino acid selected from the group | 1 to 300 mM |
| consisting of: arginine, alanine, phenylalanine, | |
| glycine, glutamine, glutamic acid, methionine, | |
| and lysine | |
| A sugar selected from the group consisting of: | 1 to 500 mM |
| dextrose, fructose, galactose, glucose, raffinose, | |
| trehalose, or sucrose | |
| As poly(ethylene oxide) and poly(propylene | 0.01 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3f
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| An Amino acid selected from the group | 1 to 300 mM |
| consisting of: arginine, alanine, phenylalanine, | |
| glycine, glutamine, glutamic acid, methionine, | |
| and lysine | |
| A sugar selected from the group consisting of: | 1 to 500 mM |
| dextrose, fructose, galactose, glucose, | |
| raffinose, trehalose, and sucrose | |
| As poly(ethylene oxide) and poly(propylene | 0.01 g/L to 50 g/L |
| oxide) block copolymer: poloxamer 188 | |
| and/or | |
| As protein agent: human serum albumin or | 0.05 g/L to 50 g/L |
| recombinant human albumin | |

TABLE 3g
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| Tris buffer | 1 to 100 mM |
| An Amino acid selected from the group | 1 to 300 mM |
| consisting of: arginine, alanine, phenylalanine, | |
| glycine, glutamine, glutamic acid, methionine, | |
| and lysine | |
| A sugar selected from the group consisting of: | 1 to 500 mM |
| dextrose, fructose, galactose, glucose, raffinose, | |
| trehalose, and sucrose | |
| As poly(ethylene oxide) and poly(propylene | 0.01 g/L to 10 g/L |
| oxide) block copolymer: poloxamer 188 | |
| and/or | |
| As protein agent: human serum albumin or | 0.05 g/L to 10 g/L |
| recombinant human albumin | |

TABLE 3h
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Buffer selected from the group consisting of: | 1 to 100 mM |
| acetate, citrate, histidine, succinate, HEPES, | |
| tartrate, phosphate, citrate/phosphate, lactate, | |
| and Tris; preferably Tris | |
| Arginine | 1 to 300 mM |
| A sugar selected from the group consisting of: | 1 to 500 mM |
| dextrose, fructose, galactose, glucose, raffinose, | |
| trehalose, and sucrose | |
| As poly(ethylene oxide) and poly(propylene | 0.01 g/L to 10 g/L |
| oxide) block copolymer: poloxamer 188 | |
| and/or | |
| As protein agent: human serum albumin or | 0.05 g/L to 10 g/L |
| recombinant human albumin | |

TABLE 3i
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Buffer selected from the group consisting of: | 1 to 100 mM |
| acetate, citrate, histidine, succinate, HEPES, | |
| tartrate, phosphate, citrate/phosphate, lactate, | |
| and Tris; preferably Tris | |
| An Amino acid selected from the group | 1 to 300 mM |
| consisting of: arginine, alanine, phenylalanine, | |
| glycine, glutamine, glutamic acid, methionine, | |
| and lysine | |
| Trehalose, or sucrose | 1 to 500 mM |
| As poly(ethylene oxide) and poly(propylene | 0.01 g/L to 10 g/L |
| oxide) block copolymer: poloxamer 188 | |
| and/or | |
| As protein agent: human serum albumin or | 0.05 g/L to 10 g/L |
| recombinant human albumin | |

TABLE 3j
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| Tris | 1 to 100 mM |
| Arginine | 1 to 300 mM |
| A sugar selected from the group consisting of: | 1 to 500 mM |
| dextrose, fructose, galactose, glucose, | |
| raffinose, trehalose, and sucrose | |
| As poly(ethylene oxide) and poly(propylene | 0.01 g/L to 10 g/L |
| oxide) block copolymer: poloxamer 188 | |
| and/or | |
| As protein agent: human serum albumin or | 0.05 g/L to 10 g/L |
| recombinant human albumin | |

TABLE 3k
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| Tris buffer | 1 to 100 mM |
| An Amino acid selected from the group | 1 to 300 mM |
| consisting of: arginine, alanine, phenylalanine, | |
| glycine, glutamine, glutamic acid, methionine, | |
| and lysine | |
| Trehalose or sucrose | 1 to 500 mM |
| As poly(ethylene oxide) and poly(propylene | 0.01 g/L to 10 g/L |
| oxide) block copolymer: poloxamer 188 | |
| and/or | |
| As protein agent: human serum albumin or | 0.05 g/L to 10 g/L |
| recombinant human albumin | |

TABLE 3l
Typical concentration ranges of the components of a
formulation, preferably a liquid or frozen liquid formulation

| Component | Concentration Range |
|---|---|
| An enveloped virus, preferably a vesiculovirus | 1 × 10⁵ TCID₅₀/mL to |
| or a vesicular stomatitis virus, more preferably | 1 × 10¹² TCID₅₀/mL |
| VSV-GP | |
| A Buffer selected from the group consisting of: | 1 to 100 mM |
| acetate, citrate, histidine, succinate, HEPES, | |
| tartrate, phosphate, citrate/phosphate, lactate, | |
| and Tris; preferably Tris | |
| Arginine | 1 to 300 mM |
| Trehalose or sucrose | 1 to 500 mM |
| As poly(ethylene oxide) and poly(propylene | 0.01 g/L to 10 g/L |
| oxide) block copolymer: poloxamer 188 | |
| and/or | |
| As protein agent: human serum albumin or | 0.05 g/L to 10 g/L |
| recombinant human albumin | |

In its broadest form the invention relates to a pharmaceutical composition comprising an enveloped virus and a protein agent and/or a poly(ethylene oxide)/poly(propylene oxide) block copolymer. The phrase at least one of a buffer, a sugar, or an amino acid means that said formulation must contain at least one of those components but can also contain two or all three of those components. For example, a formulation comprising at least one of a buffer, a sugar, or an amino acid may contain a buffer; a sugar; an amino acid; a buffer and a sugar; a buffer and an amino acid; a buffer and a sugar and an amino acid; or a sugar and an amino acid. It will be understood, that said formulations may comprise also e.g., two or more amino acids, two or more sugars, etc. Preferably, said formulations are provided as liquid or frozen liquid formulations.

In any of the above mentioned formulations, preferably liquid or frozen liquid formulation embodiments the poloxamer concentration can be in any concentration range between 0.01-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.01-40 g/L, 0.01-30 g/L, 0.01-20 g/L, 0.01-10 g/L, 0.01-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L. Preferably the poloxamer is a poloxamer 188 and the concentration is in a range between 0.1-10 g/L.

In any of the above mentioned formulations, preferably liquid or frozen liquid formulation embodiments the protein agent concentration can be in any concentration range between 0.05-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.05-40 g/L, 0.05-30 g/L, 0.05-20 g/L, 0.05-10 g/L, 0.05-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L. Preferably, the protein agent is recombinant human albumin or human serum albumin and the concentration is in a range between 0.5-10 g/L.

In any of the above-mentioned formulations, preferably liquid or frozen liquid formulation embodiments the buffer concentration can be in any concentration range between 1-100 mM, 1-90 mM, 1-80 mM, 1-70 mM, 1-60 mM, 1-50 mM, 1-40 mM, 1-30 mM, 1-20 mM, or 1-10 mM. Preferably, the formulation, preferably liquid or frozen liquid formulation comprises a Tris buffer and more preferably a Tris buffer in a concentration between 5 and 50 mM.

In any of the above mentioned formulations, preferably liquid or frozen liquid formulation embodiments the sugar concentration can be in any concentration range between 10-1000 mM, 10-900 mM, 10-800 mM, 10-700 mM, 10-600 mM, 10-500 mM, 10-400 mM, 10-300 mM, 10-200 mM, 20-1000 mM, 20-900 mM, 20-800 mM, 20-700 mM, 20-600 mM, 20-500 mM, 20-400 mM, 20-300 mM, 20-200 mM, 30-1000 mM, 30-900 mM, 30-800 mM, 30-700 mM, 30-600 mM, 30-500 mM, 30-400 mM, 30-300 mM, 30-200 mM, 40-1000 mM, 40-900 mM, 40-800 mM, 40-700 mM, 40-600 mM, 40-500 mM, 40-400 mM, 40-300 mM, 40-200 mM, 50-1000 mM, 50-900 mM, 50-800 mM, 50-700 mM, 50-600 mM, 50-500 mM, 50-400 mM, 50-300 mM, or 50-200 mM. Preferably, the sugar concentration is between 50 mM and 300 mM, between 50 mM and 250 mM, between 50 mM or 200 mM. Preferably, the formulation, preferably liquid or frozen liquid formulation comprises a sugar, such as dextrose, fructose, galactose, glucose, raffinose, trehalose, or sucrose. More preferably the sugar is trehalose and in a concentration between 50 and 250 mM.

The pH of the above-mentioned formulations, preferably liquid or frozen liquid formulation embodiments are usually kept in a range between 5 to 9, or between 6 to 9, or between 6 to 8, or between 6.5 to 8.5, or between 6.5 to 8.0, preferably between 7.0 and 8.0.

In any of the above-mentioned formulations, preferably liquid or frozen liquid formulation embodiments the enveloped virus is preferably a vesiculovirus, more preferably a vesicular stomatitis virus and most preferred a vesicular stomatitis virus having the glycoprotein G replaced with the glycoprotein GP of the lymphocytic choriomeningitis virus (LCMV). In any of the above-mentioned formulations, preferably liquid or frozen liquid formulation embodiments the virus concentration can be in any concentration range between 1×10⁵ TCID₅₀/mL to 1×10¹² TCID₅₀/mL or in a concentration of at least 1×10⁵ TCID₅₀/mL, at least 1×10⁶ TCID₅₀/mL, at least 1×10⁷ TCID₅₀/mL, at least 1×10⁸ TCID₅₀/mL, or at least 1×10⁹ TCID₅₀/mL. Other ranges include between 1×10⁶ TCID₅₀/mL to 1×10¹² TCID₅₀/mL, between 1×10⁶ TCID₅₀/mL to 1×10¹¹ TCID₅₀/mL and the like.

In a preferred embodiment, a liquid or frozen liquid formulation comprises an enveloped virus, about 10 mM Tris, about 150 mM arginine, about 100 mM trehalose, about 0.5 mg/mL poloxamer 188, about 2 mg/ml rHA, a pH between 7.0 and 8.0, preferably about pH 7.5. Preferably, the pH of the composition is adjusted with phosphoric acid or sodium phosphate.

In a preferred embodiment, a liquid or frozen liquid formulation comprises an enveloped virus, about 8-12 mM Tris, about 140-160 mM arginine, about 90-110 mM trehalose, about 0.4-0.6 mg/mL poloxamer 188, about 1.5-2.5 mg/ml rHA, a pH between 7.0 and 8.0, preferably about pH 7.5. Preferably, the pH of the composition is adjusted with phosphoric acid or sodium phosphate.

In another embodiment, a liquid or frozen liquid formulation comprises an enveloped virus, about 150 mM arginine, about 0.5 mg/ml poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 140-160 mM arginine, about 0.4-0.6 mg/mL poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 10 mM Tris, about 150 mM arginine, about 0.5 mg/ml poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 8-12 mM Tris, about 140-160 mM arginine, about 0.4-0.6 mg/mL poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 150 mM arginine, about 0.5 mg/mL poloxamer 188, about 2 mg/mL rHA, a pH between 7.0 and 8.0, preferably about pH 7.5. Preferably, the pH of the composition is adjusted with phosphoric acid.

In another embodiment, a liquid or frozen liquid formulation comprises an enveloped virus, about 140-160 mM arginine, about 0.4-0.6 mg/mL poloxamer 188, about 1.5-2.5 mg/mL rHA, a pH between 7.0 and 8.0, preferably about pH 7.5. Preferably, the pH of the composition is adjusted with phosphoric acid.

In another embodiment, a liquid or frozen liquid formulation comprises an enveloped virus, about 10 mM Tris, about 150 mM arginine, about 0.5 mg/ml poloxamer 188, about 2 mg/mL rHA, a pH between 7.0 and 8.0, preferably about pH 7.5. Preferably, the pH of the composition is adjusted with phosphoric acid.

In another embodiment, a liquid or frozen liquid formulation comprises an enveloped virus, about 8-12 mM Tris, about 140-160 mM arginine, about 0.4-0.6 mg/mL poloxamer 188, about 1.5-2.5 mg/ml rHA, a pH between 7.0 and 8.0, preferably about pH 7.5. Preferably, the pH of the composition is adjusted with phosphoric acid.

In another embodiment, a liquid or frozen liquid formulation comprises an enveloped virus, about 150 mM arginine, about 100 mM Trehalose, about 0.5 mg/mL poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 140-160 mM arginine, about 90-110 mM Trehalose, about 0.4-0.6 mg/mL poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 10 mM Tris, about 150 mM arginine, about 100 mM Trehalose, about 0.5 mg/mL poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 8-12 mM Tris, about 140-160 mM arginine, about 90-110 mM Trehalose, about 0.4-0.6 mg/mL poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 150 mM arginine, about 100 mM Trehalose, about 2 mg/ml rHA, about 0.5 g/L poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 140-160 mM arginine, about 90-110 mM Trehalose, about 1.5-2.5 mg/mL rHA, about 0.4-0.6 g/L poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 10 mM Tris, about 150 mM arginine, about 100 mM Trehalose, about 2 mg/mL rHA, about 0.5 g/L poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 8-12 mM Tris, about 140-160 mM arginine, about 90-110 mM Trehalose, about 1.5-2.5 gm/mL rHA, about 0.4-0.6 g/L poloxamer 188, a pH between 7.0 and 8.0, preferably about pH 7.5. Preferably, the pH of the composition is adjusted with phosphoric acid.

In another embodiment, a liquid or frozen liquid formulation comprises an enveloped virus, about 8-12 mM Tris, about 140-160 mM arginine, about 90-110 mM Trehalose, about 4-6 mg/ml rHA, a pH between 7.0 and 8.0, preferably about pH 7.5. Preferably, the pH of the composition is adjusted with phosphoric acid.

In another embodiment, a liquid or frozen liquid formulation comprises an enveloped virus, about 10 mM Tris, about 150 mM arginine, about 200 mM Trehalose, about 5 g/L rHA, about 5 g/L poloxamer 188, a pH between 7.0 and 8.0, preferably about pH 7.5. Preferably, the pH of the composition is adjusted with phosphoric acid.

TABLE 4
Exemplary formulations

| | | | | Poly(ethylene oxide) and | | |
|---|---|---|---|---|---|---|
| | | Sugar / Sugar | | poly(propylene oxide) block | | pH |
| Formula | Buffer | alcohol | Amino acids | copolymer and/or Protein agent | Others | (adjustment) |
| I | 20 mM TRIS | 150 mM Sucrose | 20 mM Glutamic acid | 5 g/L rHA | — | 7.4 HCl/NaOH |
| | | 50 mM Mannitol | | | | |
| | | 50 mM Sorbitol | | | | |
| II | 20 mM TRIS | 100 mM Sucrose | 20 mM Glutamic acid | — | — | 7.4 HCl/NaOH |
| | | 50 mM Mannitol | | | | |
| | | 50 mM Sorbitol | | | | |
| III | 20 mM TRIS | 100 mM Sucrose | 120 mM Alanine | — | 0.5 mM EDTA | 7.4 HCl/NaOH |
| IV | 20 mM TRIS | 100 mM Sucrose | 100 mM Glycine | — | 0.02 g/L PS20 | 7.4 HCl/NaOH |
| | | | 10 mM Methionine | | | |
| V | 20 mM TRIS | 100 mM Sucrose | 100 mM Phenylalanine | — | 50 mM NaCl | 7.4 Phosphoric |
| | | 7.5 mM Dextran | | | | acid / salts |
| VI | 20 mM TRIS | 200 mM Trehalose | 80 mM Alanine | — | — | 7.4 HCl/NaOH |
| | | 50 mM Mannitol | | | | |
| | | 50 mM Sorbitol | | | | |
| VII | 20 mM TRIS | 200 mM Trehalose | 80 mM Arginine | — | — | 7.4 Phosphoric |
| | | | | | | acid / salts |
| VIII | 20 mM TRIS | 200 mM Trehalose | 80 mM Arginine | — | 0.02 g/L PS20 | 7.4 Phosphoric |
| | | | | | | acid / salts |
| IX | 20 mM TRIS | 200 mM Trehalose | 120 mM Phenylalanine | — | — | 7.4 HCl/NaOH |
| | | 5 mM Sorbitol | | | | |
| X | 20 mM TRIS | 150 mM Sucrose | 20 mM Glutamic acid | 0.02 g/L Poloxamer 188 | 50 mM NaCl | 7.4 HCl/NaOH |
| XI | 20 mM TRIS | 150 mM Sucrose | 120 mM Alanine | — | — | 7.4 HCl/NaOH |
| XII | 20 mM TRIS | 125 mM Sucrose | 100 mM Phenylalanine | — | — | 7.4 Phosphoric |
| | | 125 mM Mannitol | | | | acid / salts |
| XIII | 20 mM TRIS | 150 mM Sucrose | 100 mM Phenylalanine | — | 7.5 g/L Dextran 70 | 7.4 Phosphoric |
| | | | | | | acid / salts |

TABLE 4a
Exemplary formulations

| | | | | | Poloxamer | |
|---|---|---|---|---|---|---|
| | | Tris | Arginine | Trehalose | 188 | rHA |
| Formulation | pH | [mM] | [mM] | [mM] | [mg/mL] | [mg/mL] |
| 1 | 7.5 | 10 | 150 | 100 | 0 | 5 |
| 2 | 7.5 | 10 | 150 | 200 | 5 | 5 |
| 3 | 7.5 | 10 | 100 | 100 | 0 | 2 |
| 4 | 7.5 | 10 | 100 | 0 | 0 | 10 |
| 5 | 7.5 | 10 | 300 | 200 | 5 | 10 |
| 6 | 7.5 | 10 | 150 | 0 | 2.5 | 10 |
| 7 | 7.5 | 10 | 300 | 100 | 0 | 10 |
| 8 | 7.5 | 10 | 300 | 200 | 2.5 | 5 |
| 9 | 7.5 | 10 | 150 | 200 | 0 | 10 |
| 10 | 7.5 | 10 | 100 | 0 | 5 | 5 |
| 11 | 7.5 | 10 | 100 | 200 | 2.5 | 10 |
| 12 | 7.5 | 10 | 150 | 100 | 2.5 | 5 |
| 13 | 7.5 | 10 | 150 | 100 | 5 | 10 |
| 14 | 7.5 | 10 | 150 | 0 | 2.5 | 2 |
| 15 | 7.5 | 10 | 300 | 100 | 5 | 2 |
| 16 | 7.5 | 10 | 150 | 100 | 2.5 | 5 |
| 17 | 7.5 | 10 | 300 | 200 | 0 | 2 |
| 18 | 7.5 | 10 | 300 | 0 | 0 | 5 |
| 19 | 7.5 | 10 | 300 | 0 | 5 | 10 |
| 20 | 7.5 | 10 | 100 | 200 | 5 | 2 |

In another embodiment, in any of the formulations shown in Table 4a the buffer Tris may be omitted or may be replaced with any other buffer as described herein.

### Further Aspects:

In a second aspect, the invention relates to a pharmaceutical composition comprising an enveloped virus, a buffer, an amino acid, (optionally) a sugar, AND a protein agent and/or a poly(ethylene oxide)/poly(propylene oxide) block copolymer.

In one embodiment relating to the second aspect, the amino acid is selected from the group consisting of alanine, arginine, phenylalanine, glutamic acid, glycine, methionine, lysine, or glutamine, preferably arginine.

In one embodiment relating to the second aspect, the composition is substantially free of chloride, preferably substantially free of NaCl.

In one embodiment relating to the second aspect, the pH of the composition is between 5 to 9, or between 6 to 9, or between 6.5 to 8.5, or between 6.5 to 8.0, preferably between 7.0 and 8.0. In a related embodiment, the pH of the composition is adjusted with phosphoric acid or sodium phosphate.

In one embodiment relating to the second aspect, the buffer is selected from the group consisting of acetate, citrate, histidine, succinate, HEPES, tartrate, phosphate, citrate/phosphate, lactate, and Tris, preferably Tris.

In one embodiment relating to the second aspect, the sugar is sucrose or trehalose, preferably trehalose.

In one embodiment relating to the second aspect, the composition further comprises one or more sugar alcohols. In a related embodiment, the one or more sugar alcohol(s) is/are selected from the group consisting of: mannitol, sorbitol, xylitol, maltitol, maltitol symp, lactitol, inositol, glycerol erythritol, isomalt, or hydrogenated starch hydroxylate. In a further related embodiment, the one or more sugar alcohol(s) is/are mannitol and/or sorbitol, preferably a combination of mannitol and sorbitol.

In one embodiment relating to the second aspect, the poly(ethylene oxide) and poly(propylene oxide) block copolymer is a poloxamer, preferably a pharmaceutically acceptable poloxamer, more preferably poloxamer 188.

In one embodiment relating to the second aspect, the protein agent is albumin, gelatin, preferably human serum albumin or recombinant human albumin.

In one embodiment relating to the second aspect, the composition comprises both a protein agent and a poly(ethylene oxide)/poly(propylene oxide) block copolymer. In a related embodiment, the protein agent is recombinant human albumin and the poly(ethylene oxide)/poly(propylene oxide) block copolymer is poloxamer 188.

In one embodiment relating to the second aspect or any of its embodiments, the pharmaceutical composition comprises an enveloped virus, a buffer in a concentration between 1 mM to 100 mM, arginine in a concentration between 10 mM to 500 mM, a sugar in a concentration between 10 mM to 1000 mM, AND poloxamer 188 in a concentration between 0.01 g/L to 50 g/L and/or recombinant human albumin in a concentration between 0.1 g/L to 50 g/L.

In one embodiment relating to the second aspect or any of its embodiments, the pharmaceutical composition comprises an enveloped virus; a buffer in a concentration between 1 mM to 100 mM selected from the group consisting of acetate, citrate, histidine, succinate, HEPES, tartrate, phosphate, citrate/phosphate and Tris, preferably a Tris buffer; arginine in a concentration between 10 mM to 500 mM; a sugar in a concentration between 10 mM to 1000 mM selected from the group consisting of dextrose, fructose, galactose, glucose, raffinose, trehalose, or sucrose, preferably trehalose; AND poloxamer 188 in a concentration between 0.01 g/L to 50 g/L and/or recombinant human albumin in a concentration between 0.1 g/L to 50 g/L.

In a third aspect, the invention relates to a pharmaceutical composition comprising an enveloped virus, a Tris buffer, arginine, (optionally) a sugar, AND poloxamer 188 and/or recombinant human albumin.

In one embodiment relating to the third aspect, the pharmaceutical composition comprises an enveloped virus, a Tris buffer, arginine, (optionally) a sugar, AND poloxamer 188 and recombinant human albumin.

In one embodiment relating to the third aspect, the poloxamer 188 is in a concentration between 0.01 g/L to 50 g/L.

In one embodiment relating to the third aspect, the recombinant human albumin is in a concentration between 0.1 g/L to 50 g/L.

In a fourth aspect, the invention relates to a pharmaceutical composition comprising an enveloped virus; a buffer in a concentration between 1 mM to 100 mM selected from the group consisting of acetate, citrate, histidine, succinate, HEPES, tartrate, phosphate, citrate/phosphate and Tris, preferably a Tris buffer; arginine in a concentration between 10 mM to 500 mM; a sugar in a concentration between 10 mM to 1000 mM selected from the group consisting of dextrose, fructose, galactose, glucose, raffinose, trehalose, or sucrose, preferably trehalose; AND poloxamer 188 in a concentration between 0.01 g/L to 50 g/L and/or recombinant human albumin in a concentration between 0.1 g/L to 50 g/L.

In an embodiment relating to the fourth aspect, the pharmaceutical composition comprises an enveloped virus, a Tris buffer in a concentration between 1 mM to 100 mM, arginine in a concentration between 10 mM to 500 mM, trehalose in a concentration between 10 mM to 1000 mM, AND poloxamer 188 in a concentration between 0.01 g/L to 50 g/L and/or recombinant human albumin in a concentration between 0.1 g/L to 50 g/L.

In an embodiment relating to any of the foregoing aspects and their embodiments, the enveloped virus is a rhabdoviridae, preferably a vesiculovirus or vesicular stomatitis virus (VSV). In a related embodiment, the enveloped virus is a recombinant vesicular stomatitis virus (VSV), wherein the gene coding for the glycoprotein G of the vesicular stomatitis virus is replaced by the gene coding for the glycoprotein GP of LCMV, and/or the glycoprotein G is replaced by the glycoprotein GP of LCMV.

In an embodiment relating to any of the foregoing aspects and their embodiments, the pharmaceutical composition is a liquid or frozen liquid pharmaceutical composition. In a related embodiment, the composition is frozen and stored at a temperature of about −80° C., −70° C., −60° C., −50° C., −40° C., −35° C., −30° C., −25° C., −20° C., −15° C., −10° C., or −5° C.

In a fifth aspect, the invention relates to a product produced by lyophilizing the liquid pharmaceutical composition.

In a sixth aspect, the invention relates to a dry pharmaceutical composition produced by a method comprising removing water from a pharmaceutical composition, said composition comprising an enveloped virus, a Tris buffer in a concentration between 1 mM and 100 mM, trehalose in a concentration between 10 mM and 1000 mM, arginine in a concentration between 10 mM to 500 mM, AND poloxamer 188 in a concentration between 0.01 g/L and 50 g/L and/or recombinant human albumin in a concentration between 0.1 g/L and 50 g/L.

In an embodiment relating to the sixth aspect, the pharmaceutical composition is frozen to obtain a pharmaceutical composition comprising ice prior to removing water. In a related embodiment, the method further comprises placing the liquid pharmaceutical composition in a vacuum under controlled temperatures and pressure to remove the water. In a further related embodiment, the method is lyophilization. In another related embodiment, the dry pharmaceutical composition comprises less than about (0.5%-5%) w/w water.

In another embodiment, the pharmaceutical composition comprises water and the product of the fifth aspect or water and the dry pharmaceutical composition of the sixth aspect and any its embodiments.

### EXAMPLES

### Methods

### Formulations and Tested Viruses

The tested viruses were vesicular stomatitis viruses having the wild-type glycoprotein G replaced with the glycoprotein GP of the lymphocytic chorio-meningitis virus; such virus is named subsequently VSV-GP. In some instances, the VSV-GP encodes additionally for a cargo and such virus is named subsequently VSV-GP-Cargo1/2/or 3. The virus material VSV-GP or VSV-GP-cargo1/2/or 3 is used undiluted at a concentration of approx. 5×10⁹ TCID₅₀/ml. To transfer the virus in specific formulations, dialysis cassettes (Slide-a-Lyzer Dialysis Cassette, Thermo, MWCO 10 kDa, 12 ml) are used. Samples are dialyzed three times each for 2 h slowly stirred at a temperature of 2-8° C. The last step is performed overnight. Sterile filtration is done using 0.22 μm PES filters. For macromolecules that do not pass the dialysis membrane, i.e., recombinant human albumin (rHA), dextran, and poloxamer 188, appropriate volumes of stock solutions are added after dialysis. For placebo samples, the amount of rHA and poloxamer 188 is spiked to 15 ml of the respective sterile filtered formulations.

### Method to Determine Infectivity (TCID₅₀)

### Cells and Viruses:

BHK-21 cells (#603126 (C13), CLS) are cultured in 5% CO₂ at a temperature of 37° C. Medium (GMEM #21710082, Thermo) is supplemented with 8.7% FCS and 4.3% tryptose phosphate broth. BHK-21 cells are washed with PBS and detached from the cell culture flask by incubation with TrypLE™ Select Enzyme at a temperature of 37° C. for 6-8 min. Cells are taken up in medium, counted using the Flex2 (nova biomedical) and seeded on 96-well plates.

### TCID₅₀ Assay:

In 96-well plates 104 BHK-21 cells in 100 μl supplemented GMEM are seeded per well. Following an incubation for 24 h, the adherent cells are infected with 11×0.5 log₁₀ serial dilutions of the virus or the diluent alone (negative control) before incubation for three days at a temperature of 37° C. and 5% CO₂. Brightfield images of the cell culture wells are taken with the Cytation5 Multi-Mode Imaging Reader (BioTek) using a 4× magnifying objective. Whether the imaged wells are CPE positive or negative is assessed by eye (visually). The final titer [TCID₅₀/mL] is calculated by the formula of Spearman and Karber. For each virus sample infections with serial dilutions are performed in a total of eight plates at the same day. Based on those eight replicates the TCID₅₀/mL is calculated. As assay control, (VSV-crude-harvest material, stored at a temperature of −80° C. is used and conducted at each time point in a separate 96-well-plate.

### Method to Determine Visible and Subvisible Particles

### Detection of Visible Particles by Visual Inspection (VI)

Visual inspections are performed in accordance with internal standards in a stepwise procedure. The inspection is performed by two trained examiners.

Step 1: Prior to inspection, the samples are equilibrated to room temperature in the dark.

Step 2: The vial is placed outside the inspection zone on the workbench without being swirled or inverted. The bottom of the vial as well as the meniscus of the vial is inspected for particles.

Step 3: The vial is inspected for sediment on the bottom of the vial outside of the inspection zone, directly in front of the light source. The vial is inspected in an upright position and handled carefully to avoid swirling up a potential sediment.

Step 4: The vial is inspected outside of the inspection zone, directly in front of the light source. The vial is held upright and swirled. If there is a sediment on the bottom of the vial, it would rise and form a swirlable subvisible particle sediment.

Step 5: The vial is inspected for the presence or absence of visible particles in compliance to the European Pharmacopoeia (9^{th} edition; monograph 2.9.20) at 2,000-3,750 lux. By avoiding the formation of air bubbles, the solution is homogenized by gentle swirling and the liquid is inspected for 5 sec in front of a white and 5 sec in front of a black background in the inspection area.

Step 6: As a last step, the vial is inspected outside of the inspection zone, directly in front of the light source. The vial is carefully swirled. The sample is inspected for very small particles, which are only visible if present in high numbers.

### Detection of Sub-Visible Particles by MFI

MFI measurements are conducted using a MFI-5200 particle analyzer system equipped with a silane coated high resolution 100-μm flow cell. In brief, samples are diluted 5-fold in ADB. A pre-run volume of 0.25 ml is followed by a sample run of 0.6 ml. Between the measurements, the flow cell is rinsed with water. The background illumination is optimized by using water. MFI View System Software (MVSS) version 2-R5.0.0.43 is used to perform the measurements and MFI View Analysis Suite (MVAS) software version 1.3.0.1007 is used to analyze the samples.

### Lyophilization

Samples are lyophilized using a LCD-2-6D pilot-scale freeze dryer (Martin Christ Gefriertrocknungsanlagen GmbH, Osterode, Germany) in 2R vials (0.4 ml) with Flurotec® stoppers. The vacuum during the freeze-drying process is controlled by a capacitance manometer. Based on the calculated solid contents as well as the estimated glass transition temperature of the maximally freeze-concentrated solution (T_{g}′) a conservative lyophilization process is applied. During the freeze-drying process, product temperature, shelf temperature, condenser temperature, and chamber pressure (capacitance manometer and Pirani gauge) are monitored. The product temperature is monitored by eight Pt₁₀₀ sensors (1 sensor per formulation), which are placed in different vials located in the middle of the samples of the third shelf. At the end of the process, the stoppers of the vials are closed in the freeze-dryer under nitrogen atmosphere at a pressure of 600 mbar. After stoppering the vials, the chamber is aerated to atmospheric pressure by using nitrogen, and the samples are removed. The samples are crimp-capped after removal from the freeze-dryer, labeled, and stored at the respective storage conditions for further analysis. Lyo formulations are stored at a temperature of 25° C. or 30° C. in ICH110 Cabinets (Memmert GmbH & Co. KG, Schwabach, Germany).

### Determining the Glass Transition Temperature

The glass transition temperatures T_{g} and T_{g}′ are determined on a DSC 214 Polyma oven (Erich Netzsch GmbH & Co Holding KG, Selb, Germany). Two to ten mg lyophilized product ware weighed in aluminum pans in a glove box with controlled humidity (approx. 8% rH) and subsequently sealed in aluminum pans. T_{g} values are analyzed with Netzsch Proteus Analysis Software. All measurements are performed as duplicates and the results are calculated as the mean±standard deviation.

### Frozen Liquid Sample Preparation

Samples are frozen by placing samples in a freezer at a temperature of −70° C. or −80° C. using a CoolCell LX BioCision LLC (Larkspure, CA). The temperature is recorded by monitored by thermo-loggers. For freeze/thaw cycling, samples are removed from the freezer and placed into ICH110 Cabinets (Memmert GmbH & Co. KG, Schwabach, Germany) for at least 2 h at a temperature of 25° C.±2° C./60%±5% rH or at a temperature of 30° C.±2° C./65%±5% rH, respectively. Temperature cycling is repeated 1-, 3-, or 5-times.

### Example 1

### Initial Screening Formulations

Outline: The objectives of the initial formulation development activities were to characterize different virus formulations (Tables 5 a-c), by examining the influence of storage and freeze/thaw (F/T) cycles on infectivity (TCID₅₀) and the formation of particles in formulations designed to be stored as a liquid, a frozen liquid, or as a dry formulation, i.e., being lyophilized. In this example VSV-GP-Cargo1 was tested. In the following, WP3a formulations refer to the liquid formulations, WP3b formulations refer to the frozen liquid formulations and WP3d formulations refer to the lyophilized formulations. The general formulation was the same for the initial liquid, frozen liquid, or lyophilized formulations.

TABLE 5a
Initial liquid (WP3a) formulations

| | | General | | | | |
|---|---|---|---|---|---|---|
| | Excipient | Formulation | Liquid 1 | Liquid 2 | Liquid 3 | Liquid 4 |
| Buffer | TRIS [mM] | 20 | 20 | 20 | 20 | 20 |
| | pH 7.4 adjustment | NaOH | NaOH | NaOH | NaOH | Na₃PO₄ |
| | Sucrose [mM] | 150 | 100 | 100 | 100 | 100 |
| | Trehalose [mM] | | | | | |
| Sugar and | Dextran 70 [g/l] | | | | | 7.5 |
| polysaccharides | | | | | | |
| Sugar alcohol | Mannitol [mM] | 50 | 50 | | | |
| | Sorbitol [mM] | 50 | 50 | | | |
| Salt | NaCl [mM] | | | | | 50 |
| Amino acids | Ala [mM] | | | 120 | | |
| | Arg [mM] | | | | | |
| | Phe [mM] | | | | | 100 |
| | Glu [mM] | 20 | 20 | | | |
| | Gly [mM] | | | | 100 | |
| | Met [mM] | | | | 10 | |
| Detergent | Poloxamer 188 [g/l] | | | | | |
| | Polysorbate 20 [g/l] | | | | 0.02 | |
| Macromolecule | rHA [g/l] | 5 | | | | |
| Others | EDTA [mM] | | | 0.5 | | |

TABLE 5b
Initial frozen liquid (WP3b) formulations

| | | General | | | | |
|---|---|---|---|---|---|---|
| | Excipient | Formulation | FroLiq1 | FroLiq 2 | FroLiq3 | FroLiq 4 |
| Buffer | TRIS [mM] | 20 | 20 | 20 | 20 | 20 |
| | pH 7.4 adjustment | NaOH | NaOH | NaOH | Na3PO4 | Na3PO4 |
| Sugar and | Sucrose [mM] | 150 | | | | |
| polysaccharides | Trehalose [mM] | | 150 | 150 | 125 | 150 |
| | Dextran 70 [g/l] | | | | | 7.5 |
| Sugar alcohol | Mannitol [mM] | 50 | | | 125 | |
| | Sorbitol [mM] | 50 | | | | |
| Salt | NaCl [mM] | | 50 | | | |
| Amino acids | Ala [mM] | | | 120 | | |
| | Arg [mM] | | | | | |
| | Phe [mM] | | | | 100 | 100 |
| | Glu [mM] | 20 | 20 | | | |
| | Gly [mM] | | | | | |
| | Met [mM] | | | | | |
| Detergent | Poloxamer 188 [g/l] | | 0.02 | | | |
| | Polysorbate 20 [g/l] | | | | | |
| Macromolecule | rHA [g/l] | 5 | | | | |

TABLE 5c
Initial lyo (WP3d) formulations

| | | General | | | | |
|---|---|---|---|---|---|---|
| | Excipient | Formulation | Lyo 1 | Lyo 2 | Lyo 3 | Lyo 4 |
| Buffer | TRIS [mM] | 20 | 20 | 20 | 20 | 20 |
| | pH 7.4 adjustment | NaOH | NaOH | Na₃PO₄ | Na₃PO₄ | NaOH |
| | Sucrose [mM] | 150 | | | | |
| Sugar and | Trehalose [mM] | | 200 | 200 | 200 | 200 |
| polysaccharides | Dextran 70 [g/l] | | | | | |
| Sugar alcohol | Mannitol [mM] | 50 | 50 | | | |
| | Sorbitol [mM] | 50 | 50 | | | 5 |
| Salt | NaCl [mM] | | | | | |
| Amino acids | Ala [mM] | | 80 | | | |
| | Arg [mM] | | | 80 | 80 | |
| | Phe [mM] | | | | | 120 |
| | Glu [mM] | 20 | | | | |
| | Gly [mM] | | | | | |
| | Met [mM] | | | | | |
| Detergent | Poloxamer 188 [g/l] | | | | | |
| | Polysorbate 20 [g/l] | | | | 0.02 | |
| Macromolecule | rHA [g/l] | 5 | | | | |

### Effects of Storing Formulations at a Temperature of 5° C. or 25° C. on the Infectious Titer and SvPs in Liquid Formulations (WP3a)

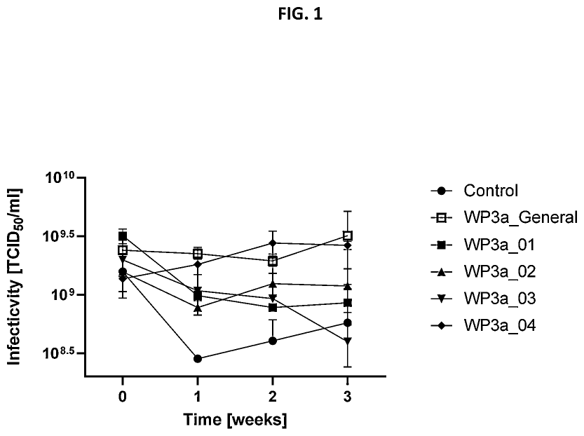

### FIGS. 1 to 4

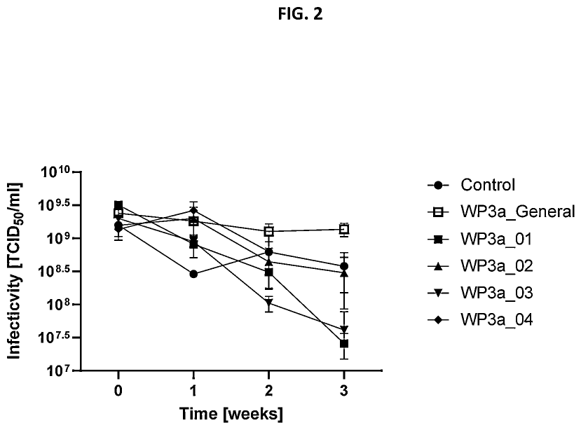

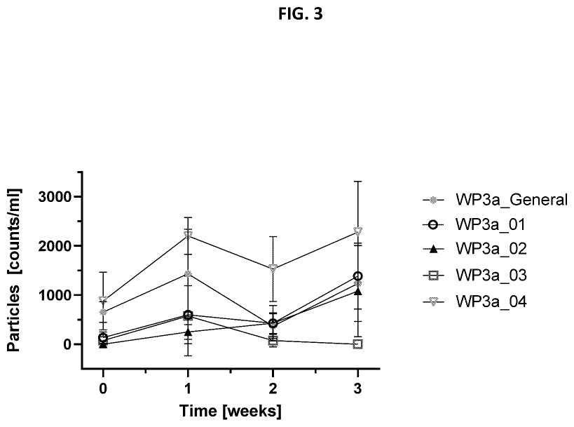

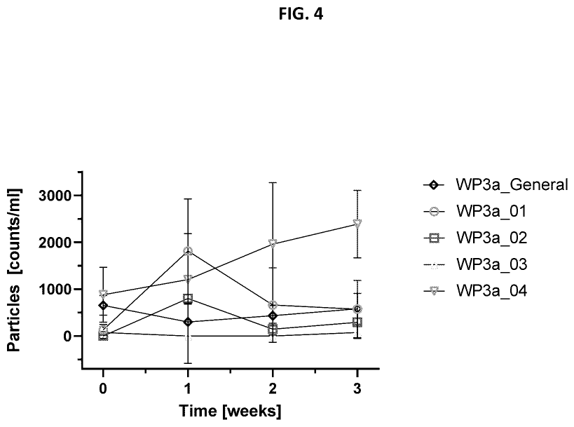

All liquid formulations (except WP3a_03 containing polysorbate at 0.02 g/L) indicated a decent stability during storage for 28 days at a temperature of 5° C. (FIG. 1) with an average loss of titer during storage of 0.2 log units and also for some formulations at a temperature of 25° C. (FIG. 2) with a loss of titer in a between 0.2-2.1 log units. The most stable formulation was WP3a_General containing rHA (at 5 g/L). All formulations had very low levels of sub visible particles (SvPs). The formulation with the lowest amount of SvPs≥10 μm after storage at a temperature of 5° C. (FIG. 3) or at 25° C. (FIG. 4) was WP3a_03 containing polysorbate.

### Effects of F/T Cycles on the Infectious Titer and Subvisible Particle Formation in Liquid Formulations (WP3a)

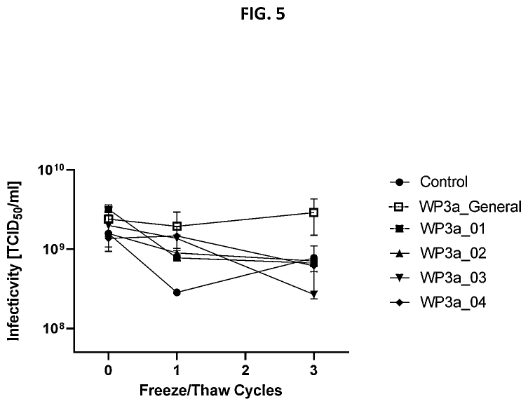

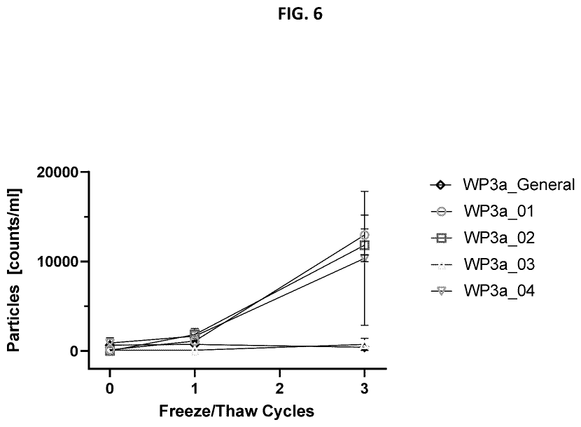

### FIGS. 5 to 6

When liquid formulations of WP3a were stressed with one or three F/T cycles from a temperature of −80° C. to +25° C. (Table 6) three F/T cycles led to higher loss of the infectious titer than one F/T cycle. The most stable formulation was WP3a_General containing rHA. The formulation containing polysorbate (WP3a_03) exhibited the highest loss in titer (FIG. 5) and was thus, no longer considered as an excipient for formulation development despite of the positive impact of polysorbate on particle formation. The negative effect of polysorbate on the stability of VSV likely stems from its effect on its phospholipid envelope, which can be easily damaged by a detergent. F/T cycling increased the amount of SvPs in all formulations except for WP3a_General containing rHA with respect to particles≥10 μM and for WP3a_03 containing polysorbate with respect to particles≥10 μM (FIG. 6).

TABLE 6
Stress and storage conditions of virus in liquid formulations
and without or with additional freeze/thaw cycles (F/T)

| | | | | Storage of virus | | |
|---|---|---|---|---|---|---|
| | F/T | t = 0 | Post | formulations in days | | |
| | (−80° C./25° C.) | (Pre F/T) | F/T | t = 7 | t = 14 | t = 28 |
| 5° C. | 0 | X | | X | X | X |
| | 1 | | X* | X | | |
| | 3 | | X* | X | | |
| 25° C./60% rH | 0 | | | X | X | X |
| | 1 | | X* | X | | |
| | 3 | | X* | X | | |

*5° C. & 25° C. samples and measurements of 1 & 3 F/T are the same.

### Effects of F/T Cycles on the Infectious Titer and Subvisible Particle Formation in Frozen Liquid Formulations (WP3b)

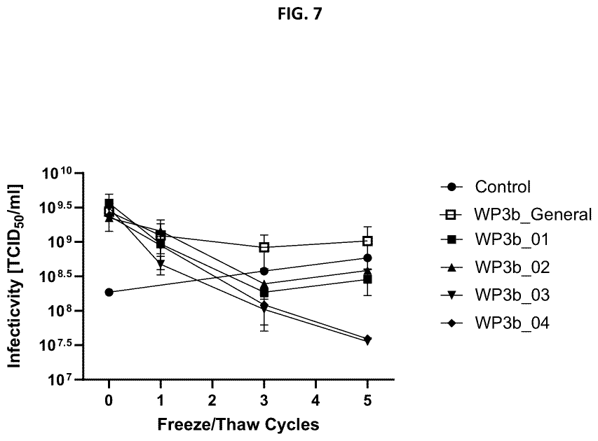

### FIGS. 7 to 10

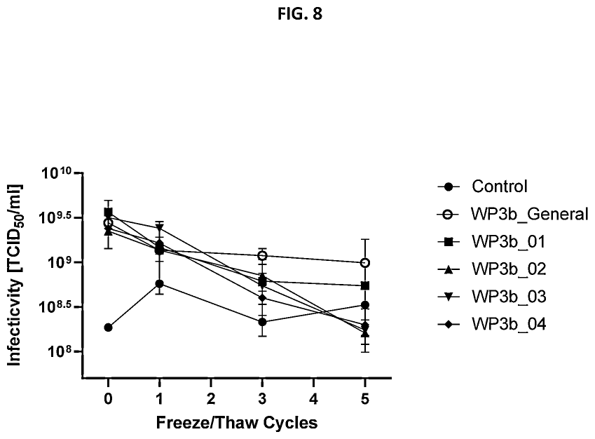

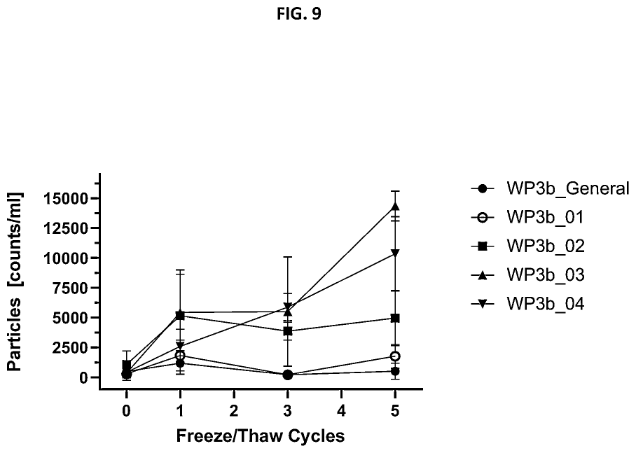

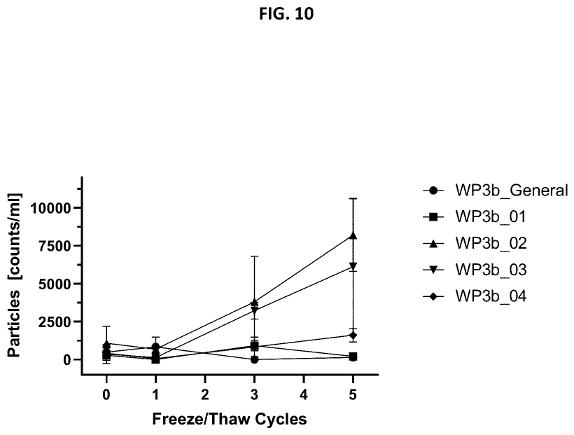

When VSV-GP was cycled from temperatures of −20° C. or −80° C. to +25° C., one, three, or five times (Table 7), the most stable formulation was again WP3a_General, containing rHA followed by the formulation containing poloxamer 188, regardless of whether the formulation was cycled from a temperature of −20° C. to +25° C. (FIG. 7) or from a temperature of −80° C. to +25° C. (FIG. 8). Only in those formulations, five F/T cycles led to no further loss of the infectious titer in comparison to three F/T cycles. Also, in those formulations, the number of SvPs≥10 UM did not increase by F/T, neither when formulations were cycled from a temperature of −20° C. to +25° C. (FIG. 9) or from a temperature of −80° C. to +25° C. (FIG. 10). The number of particles increased by more than 2-fold when cycled at a temperature of −20° C. as compared to −80° C. In view of the earlier observation, when detergents such as polysorbate damaged the envelope of VSV-GP and caused an unacceptable drop in the infectious titer, it was very surprising that the detergent poloxamer 188 did not have such an effect on the envelope and titer. In contrast, the addition of poloxamer 188 to the formulation was beneficial to both, the infectious titer and the suppression of SvPs formation.

TABLE 7
Freeze/thaw cycles at different storage conditions of virus in frozen-
liquid formulations

| | Virus formulations detected after freeze/thaw cycles | | | |
|---|---|---|---|---|
| | 0 F/T | 1 F/T | 3 F/T | 5 F/T |
| −20° C. | X | X | X | X |
| −80° C. | | X | X | X |

### Effects of Storing Formulations at a Temperature of 25° C. on the Infectious Titer and Subvisible Particle Formation in Lyophilized Formulations (WP3d)

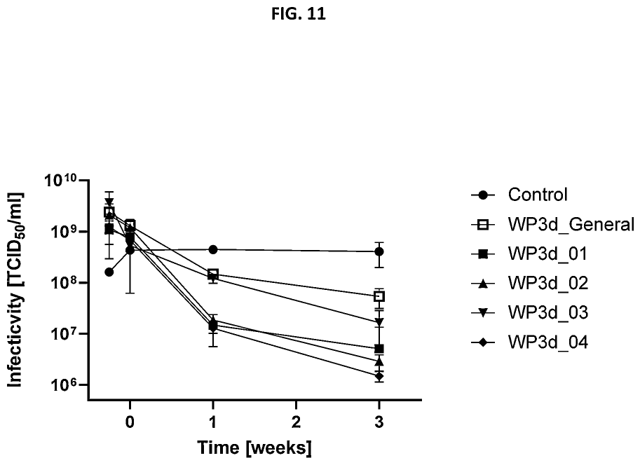

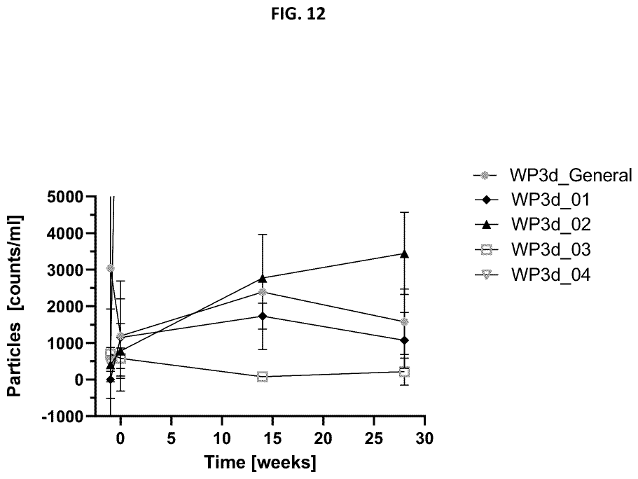

### FIGS. 11 to 12

The infectivity in all formulations was stable immediately after lyophilization but decreased almost linearly when stored at a temperature of 25° C. (FIG. 11). All formulations had very low levels of SvPs. The formulations with the lowest amount of SvPs≥10 μm (FIG. 12) were, as expected, WP3d_General, containing rHA and WP3d_03 containing polysorbate. All lyophilized formulations resulted in stable lyo cakes except WP3d_General. The reason was that in this formulation sucrose was the sugar component instead of trehalose, which was in all other lyo formulations. Since the glass transition temperature (T_{g}) value of WP3d_General was close to the storage temperature at approx. 25° C., sucrose was exchanged in the following experiments with trehalose.

Overall, WP3a_General=WP3b_General=WP3d_General, containing rHA performed well in all stress conditions and served as the basis for further lyo formulation development efforts. The composition was 20 mM Tris (titrated with NaOH), 150 mM sucrose, 50 mM mannitol, 50 mM sorbitol, 20 mM glutamic acid, 5 g/L rHA, pH 7.4.

### Example 2

### Liquid Formulation Development for VSV-GP (Step 1)

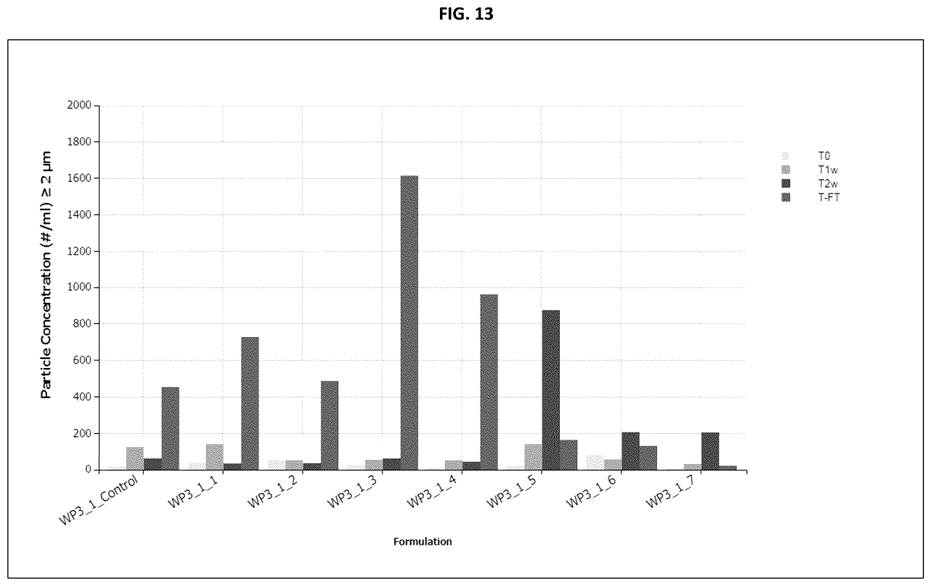

### FIGS. 13 to 16

In parallel to the aforementioned general development activities a frozen liquid formulation for VSV-GP was developed.

In a first work package the following formulations were tested.

TABLE 8
Buffers used within WP 3 step 1

| | | | | Tri- | | | | | |
|---|---|---|---|---|---|---|---|---|---|
| | | | | sodium | | Final conc. of | | | |
| | | Sodium | L- | citrate | D- | excipients | | | |
| | Tris | chloride | arginine | dihydrate | trehalose | (spiked after | | pH | pH |
| Formulation | [mmol/l] | | | | | dialysis) | | adjustment | [22° C.] |
| Control | 50 | 50 | 50 | 100 | — | — | | HCl | 7.5 |
| WP3_1_1 | 50 | 50 | 150 | — | — | — | | HCl | 7.5 |
| WP3_1_2 | 50 | 50 | 150 | — | 106 | — | | HCl | 7.5 |
| WP3_1_3 | 50 | — | 150 | — | 106 | — | | H₃PO₄ | 7.5 |
| WP3_1_4 | 50 | — | 250 | — | — | — | | H₃PO₄ | 7.5 |
| WP3_1_5 | 50 | 50 | 150 | — | — | 20 | mg/ml rHA | HCl | 7.5 |
| WP3_1_6 | 50 | 50 | 150 | — | — | 5 | mg/ml rHA | HCl | 7.5 |
| WP3_1_7 | 50 | 50 | 150 | — | — | 20 | mg/ml gelatin | HCl | 7.5 |

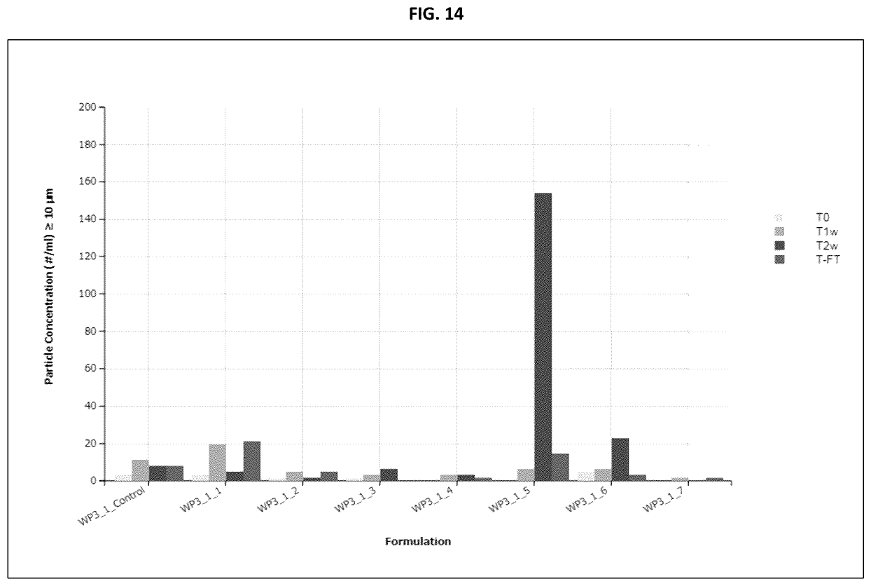

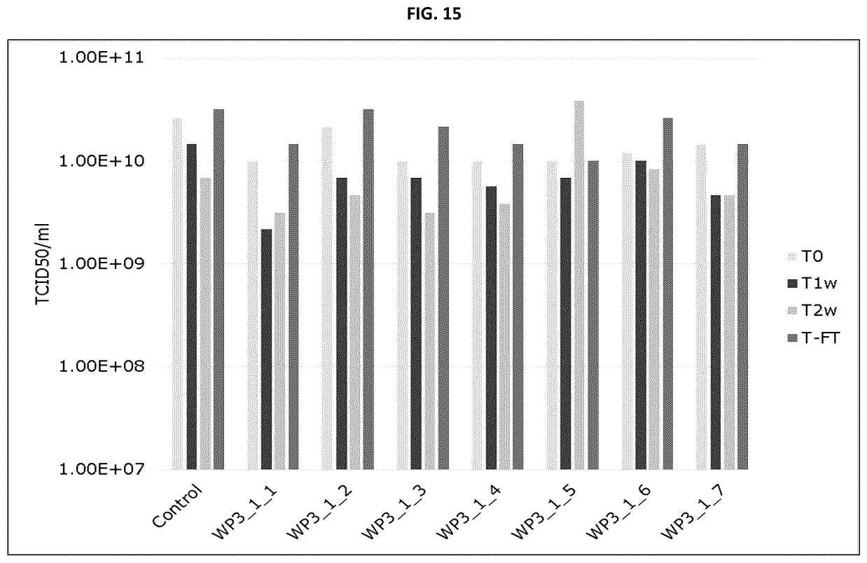

VSV-GP DP was buffer exchanged (by dialysis and spiking) into eight different formulation prototypes. The impact of a F/T cycle and incubation for up to two weeks at a temperature of 25° C. on infectivity and subvisible particle formation was analyzed.
All samples appeared turbid at all time points/conditions. Single visible particles were detected randomly and could not be linked to a certain excipient.
F/T stress increased the concentration of particles≥2 μm in all formulations (FIG. 13) and ≥10 μm (FIG. 14) that did not contain rHA or gelatin after one F/T cycle. Storage at a temperature of 25° C. for up to two weeks induced particles≥2 μm most prominently in the three rHA- or gelatin-spiked formulations, as well as in placebo vials of rHA-spiked formulations measured 23 days after filling. Since storage-induced particle formation occurred also in the absence of VSV-GP DP and the number of particles increased with the concentration of rHA, the observed particles might be attributed to the presence of rHA rather than to a VSV-GP DP/rHA interaction.
Formulations containing rHA exhibited the highest infectivity's after storage (FIG. 15), but differences were all within assay variation.

In a second work package the effect of rHA on infectious titer was again tested for VSV-GP.

For this purpose, a formulation with 10 mM Tris, 106 mM trehalose, 150 mM arginine and 5 mg/ml rHA (pH 7.5) was tested against a formulation containing the same excipients but without rHA. Formulations were either stressed via a freeze-thaw step or by storing the formulation at 25° C. for one or two weeks. Infectious virus titers of the VSV-GP samples were determined by using a TCID₅₀ assay. All samples were analyzed with n=3.

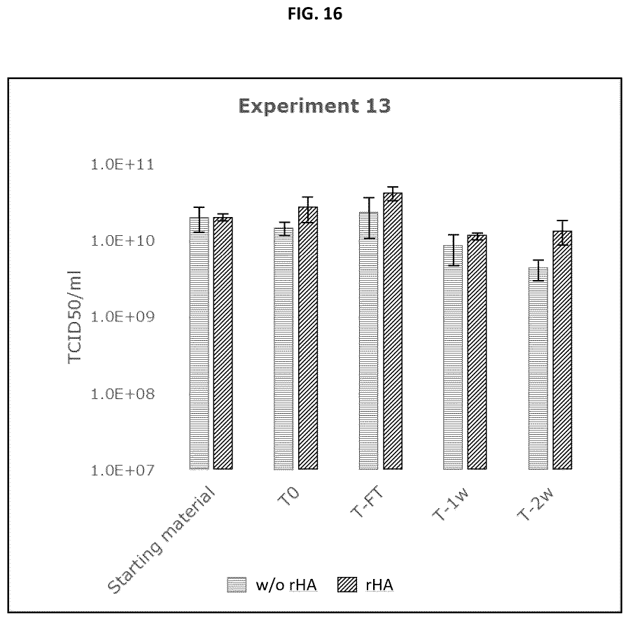

Infectious titers of the starting material with and without rHA were similar (FIG. 16). Filtration (TO) and freeze/thaw stress (T-FT) did not reduce infectious titers, irrespective of the presence of rHA. Upon storage at 25° C. and 60% r.H. for one week (T-1w) or two weeks (T-2w), a trend towards a reduction of infectious titers was determined. The decrease was more pronounced in the sample without rHA, which showed a more than ⅔ log 10 reduced infectious titer at T-2w compared to T-FT.

### Example 3

### Liquid Formulation Development for VSV-GP (Step 2)

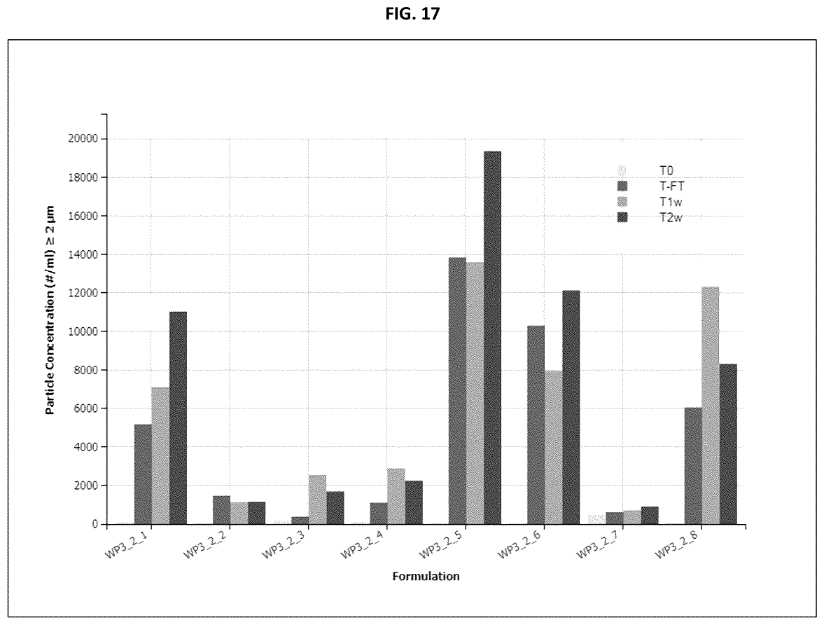

### FIGS. 17 to 19

Based on the results of the first work package a second work package (WP3 step 2) was initiated, and the following formulations were tested.

TABLE 9
Buffers used within WP 3 step 2

| | | | | | Final conc. | | | |
|---|---|---|---|---|---|---|---|---|
| | | Sodium | L- | | of rHA | | | |
| | Tris | chloride | arginine | Trehalose | (spiked after | | pH | pH |
| Formulation | [mmol/l] | | | | dialysis) | | adjustment | [22° C.] |
| WP3_2_1 | 50 | 50 | 150 | — | — | | HCl | 7.5 |
| WP3_2_2 | 50 | 50 | 150 | — | 5 | mg/ml | HCl | 7.5 |
| WP3_2_3 | 50 | 50 | 150 | — | 5 | mg/ml | H₃PO₄ | 7.5 |
| WP3_2_4 | 50 | 50 | 150 | — | 2.5 | mg/ml | H₃PO₄ | 7.5 |
| WP3_2_5 | 50 | 100 | 100 | — | — | | H₃PO₄ | 7.5 |
| WP3_2_6 | 50 | 50 | 150 | — | — | | H₃PO₄ | 7.5 |
| WP3_2_7 | 50 | 50 | 150 | 106 | 5 | mg/ml | H₃PO₄ | 7.5 |

| | | | | | | | |
|---|---|---|---|---|---|---|---|
| WP3_2_8 | 50 | 50 | 150 | 106 | — | H₃PO₄ | 7.5 |

The impact of a F/T cycle alone or a F/T cycle followed by incubation for up to two weeks at a temperature of 25° C. was analyzed by using MFI and TCID₅₀ analysis.

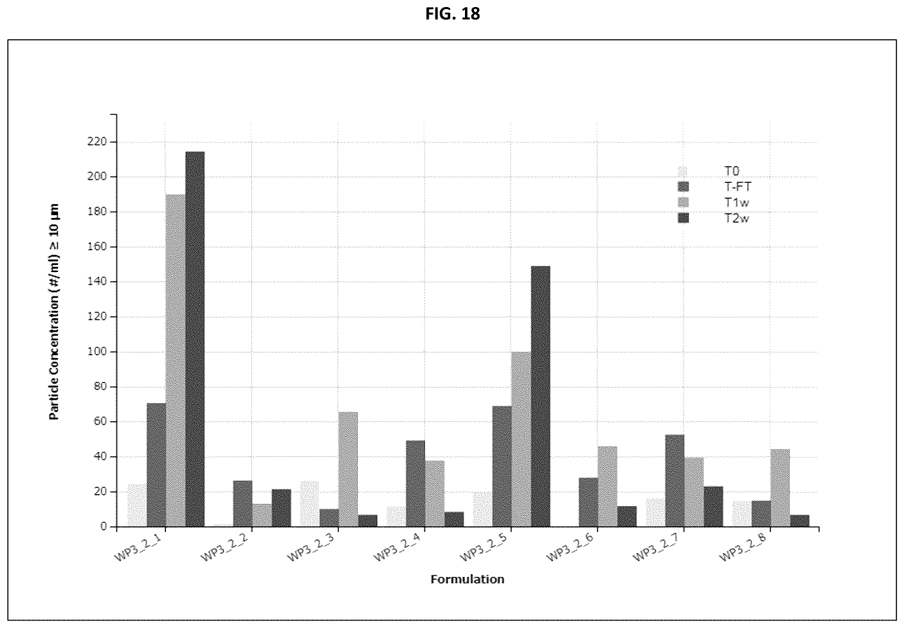

F/T stress increased the concentration of particles≥2 μm) (FIG. 17) and ≥10 μm (FIG. 18) in all formulations. Spiking of rHA largely prevented such particle formation after one F/T cycle. Subsequent incubation of the thawed rHA-spiked samples at a temperature of 25° C. for up to two weeks resulted, if at all, only in a minor increase in the number of particles≥2 μm. Formulations containing 2.5 mg/ml rHA or 5 mg/ml rHA showed comparable results, with nominally lower numbers of particles≥2 μm and ≥10 μm when using 5 mg/ml. As already observed in studies with VSV-GP the addition of trehalose slightly reduced the numbers of particles≥2 μm after F/T. The direct comparison of formulations where the pH was adjusted either with HCl or with phosphoric acid showed reduced numbers of particles≥10 μm when phosphoric acid was added instead of HCl.

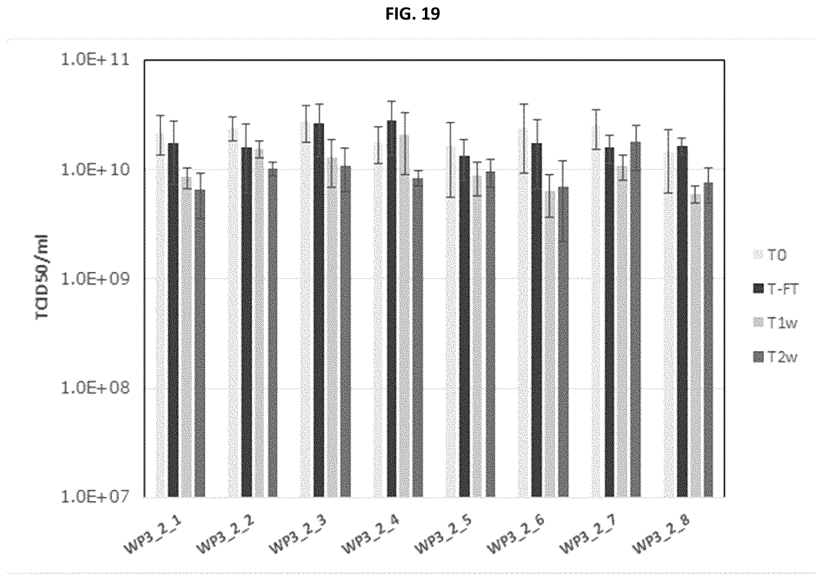

F/T stress followed by incubation at a temperature of 25° C. decreased the infectivity in all formulations. Spiking of 5 mg/ml rHA seems to be protective (FIG. 19).

### Example 8

### Liquid Formulation Development for VSV-GP-Cargo2

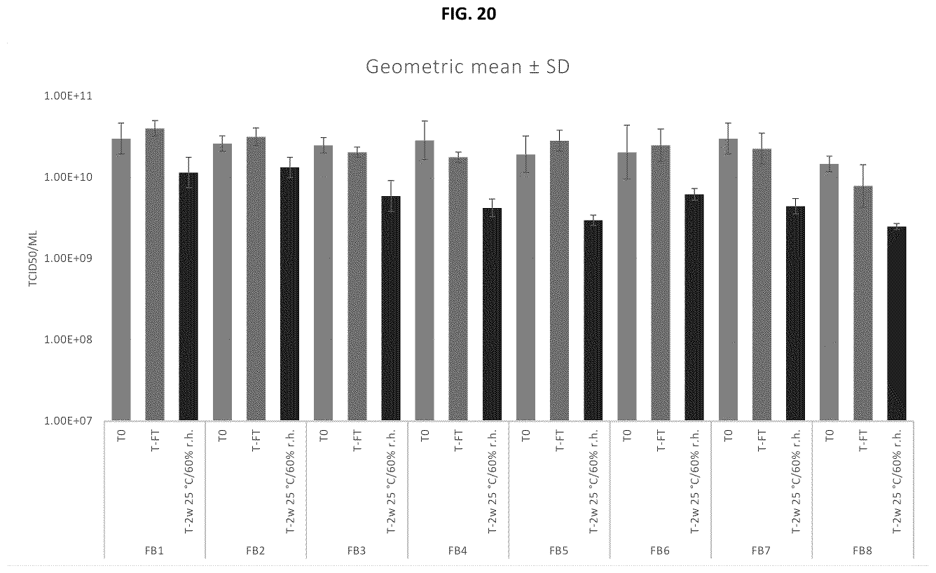

### FIG. 20

In parallel to the aforementioned general development activities a frozen liquid formulation for VSV-GP-Cargo2 (a further variant encoding for a different cargo) was developed. Development studies with VSV-GP-Cargo2 were performed to further optimize the formulation and to verify the excipients of the preliminary lead formulation. The formulation development for liquid-frozen was started with a formulation that was comprised of 9.75 mM Tris, 146 mM L-arginine, 103 mM trehalose, 5 mg/mL rHA at pH 7.5 titrated with ortho-phosphoric acid.

In different work packages the following formulations were tested.

TABLE 10
Formulations tested within WP 3A - Liquid-frozen

| | | | | | | Addition of additives | | |
|---|---|---|---|---|---|---|---|---|
| | Dialysis buffer | | | | | after dialysis² | | |
| | | L- | L- | | | | Poloxamer | Kollidon |
| Formulation | Tris | arginine | histidine | trehalose | pH¹ | rHA | 188 | 30 |
| FB1 | 10 mM | 150 mM | — | 106 mM | 7.5 | 5 mg/ml³ | — | — |
| FB2 | 10 mM | 150 mM | — | 106 mM | 7.5 | 5 mg/ml³ | 5 mg/ml | — |
| FB3 | 10 mM | 150 mM | — | 106 mM | 7.5 | — | 2 mg/ml | — |
| FB4 | 10 mM | 150 mM | — | 106 mM | 7.5 | — | 5 mg/ml | — |
| FB5 | 10 mM | 150 mM | — | 106 mM | 7.5 | — | 2 mg/ml | 10 mg/ml |
| FB6 | 10 mM | 150 mM | — | 200 mM | 7.5 | — | 5 mg/ml | — |
| FB7 | 10 mM | 50 mM | 50 mM | 200 mM | 7.5 | — | 5 mg/ml | — |
| FB8 | 50 mM | — | — | 300 mM | 7.5 | — | 5 mg/ml | — |

¹pH adjustment with ortho-phosphoric acid.
²The pH was not adjusted after addition of additives.
³Due to the addition of rHA after dialysis, dialysis buffer excipients were diluted 39:40.

TABLE 11
Formulation candidates tested within WP 4A - Liquid-frozen

| | | | | | Addition of additives | |
|---|---|---|---|---|---|---|
| | Dialysis buffer | | | | after dialysis² | |
| Formulation | Tris | L-arginine | Trehalose | pH¹ | Excipient 1 | Excipient 2 |
| FB2 | 10 mM | 150 mM | 106 mM | 7.5 | 5 mg/ml | 5 mg/ml |
| | | | | | Poloxamer 188 | rHA³ |
| FB2A | 10 mM | 150 mM | 200 mM | 7.5 | 5 mg/ml | 5 mg/ml |
| | | | | | Poloxamer 188 | rHA³ |
| FB5A | 10 mM | 150 mM | 200 mM | 7.5 | 5 mg/ml | 10 mg/ml |
| | | | | | Poloxamer 188 | Kollidon 30 |
| FB6 | 10 mM | 150 mM | 200 mM | 7.5 | 5 mg/ml | — |
| | | | | | Poloxamer 188 | |
| FB6A | 10 mM | 150 mM | 200 mM | 7.0 | 5 mg/ml | — |
| | | | | | Poloxamer 188 | |
| FB6B | 10 mM | 50 mM | 200 mM | 7.5 | 5 mg/ml | — |
| | | | | | Poloxamer 188 | |

FB[number] corresponds to the numbering in work package 3A. Modifications thereof are marked with “A” or “B”.
¹pH adjustment with ortho-phosphoric acid.
²pH was not adjusted after addition of additives.
³Due to the addition of rHA after dialysis, dialysis buffer excipients were diluted 39:40.

TABLE 12
Formulation candidates tested within WP 4D - Liquid-frozen

| | | | | | Addition of additives | |
|---|---|---|---|---|---|---|
| | Dialysis buffer | | | | after dialysis² | |
| Formulation | Tris | L-arginine | Trehalose | pH¹ | rHA | Poloxamer 188 |
| FB2A | 10 mM | 150 mM | 200 mM | 7.5 | 5 mg/ml³ | 5 mg/ml |
| FB11 | 10 mM | 150 mM | 200 mM | 7.5 | 5 mg/ml³ | 2.5 mg/ml |
| FB12 | 10 mM | 150 mM | 200 mM | 7.0 | 5 mg/ml³ | 5 mg/ml |
| FB13 | 10 mM | 150 mM | 200 mM | 7.8 | 5 mg/ml³ | 5 mg/ml |
| FB14 | 10 mM | 150 mM | 200 mM | 6.5 | 5 mg/ml³ | 5 mg/ml |

FB[number] corresponds to the numbering in work package WP 4A (FB2A) or were continued (FB11-FB14). Modifications of a formulation candidate already existing in WP 3A were marked with “A”.
¹pH adjustment was performed with ortho-phosphoric acid.
²pH was not adjusted after addition of additives.
³Due to the addition of rHA after dialysis, dialysis buffer excipients were diluted 39:40.

TABLE 13
Formulation candidates tested within WP 7A - Liquid-frozen

| | | | | | Excipient | | |
|---|---|---|---|---|---|---|---|
| | Buffer | | | | during DS | | |
| Formulation | Tris | L-arginine | Trehalose | pH | Albumin | Poloxamer 188 | Albumin |
| FB15 | 9.75 mM | 146 mM | 195 mM | 7.0 | 5 mg/ml rHA | 5 mg/ml Poloxamer 188 | |
| FB16 | 10 mM | 150 mM | 200 mM | 7.0 | | 5 mg/ml Poloxamer 188 | 5 mg/ml rHA¹ |
| FB17 | 10 mM | 150 mM | 200 mM | 7.0 | | 5 mg/ml Poloxamer 188 | |

¹Due to the addition of rHA, buffer excipients (except Poloxamer 188) were diluted 39:40.

TABLE 14
Formulation candidates tested within WP 7B - Liquid-frozen

| Formulation | Tris | L-arginine | Trehalose | pH | Poloxamer 188 | Albumin |
|---|---|---|---|---|---|---|
| FB2A | 10 mM | 150 mM | 200 mM | 7.3 | 5 mg/ml Poloxamer 188 | 5 mg/ml rHA¹ |
| FB6 | 10 mM | 150 mM | 200 mM | 7.3 | 5 mg/ml Poloxamer 188 | |

¹Due to the addition of rHA, all buffer excipients were diluted 39:40.

### Results

TABLE 15
Values of SvPs ≥2 μm or ≥10 μm following different stress
conditions, such as freeze-thaw (T-FT), storage for one or two
weeks at a temperature of 25° C. or 37° C., or after
stirring induced stress (T-stir).
Raw data were multiplied by a dilution factor of 5.

| Formu- | | Particle concentration [#/ml] | |
|---|---|---|---|
| lation | Condition | ≥2 μm | ≥10 μm |
| FB1 | T0 | 206 | 18 |
| | T-FT | 24.072 | 372 |
| | T-1 w 25° C./60% r.h. | 26.587 | 696 |
| | T-1 w 37° C./75% r.h. | 36.646 | 1.817 |
| | T-2 w 25° C./60% r.h. | 30.411 | 591 |
| | T-2 w 37° C./75% r.h. | 46.370 | 865 |
| | T-stir | 130.635 | 821 |
| | T-unstir | 31.537 | 329 |
| | Placebo | 701 | 44 |
| FB2 | T0 | 351 | 3 |
| | T-FT | 107 | 7 |
| | T-1 w 25° C./60% r.h. | 356 | 51 |
| | T-1 w 37° C./75% r.h. | 308 | 11 |
| | T-2 w 25° C./60% r.h. | 79 | 8 |
| | T-2 w 37° C./75% r.h. | 390 | 3 |
| | T-stir | 32.037 | 57 |
| | T-unstir | 313 | 21 |
| | Placebo | 203 | 5 |
| FB3 | T0 | 283 | 3 |
| | T-FT | 107 | 3 |
| | T-1 w 25° C./60% r.h. | 365 | 23 |
| | T-1 w 37° C./75% r.h. | 6.323 | 34 |
| | T-2 w 25° C./60% r.h. | 305 | 5 |
| | T-2 w 37° C./75% r.h. | 14.242 | 131 |
| | T-stir | 95.875 | 38 |
| | T-unstir | 462 | 28 |
| | Placebo | 97 | 3 |
| FB4 | T0 | 170 | 2 |
| | T-FT | 164 | 3 |
| | T-1 w 25° C./60% r.h. | 1.973 | 167 |
| | T-1 w 37° C./75% r.h. | 5.566 | 75 |
| | T-2 w 25° C./60% r.h. | 120 | 5 |
| | T-2 w 37° C./75% r.h. | 5.495 | 52 |
| | T-stir | 48.589 | 31 |
| | T-unstir | 285 | 38 |
| | Placebo | 111 | 13 |
| FB5 | T0 | 92 | 8 |
| | T-FT | 138 | 2 |
| | T-1 w 25° C./60% r.h. | 111 | 3 |
| | T-1 w 37° C./75% r.h. | 3.531 | 39 |
| | T-2 w 25° C./60% r.h. | 388 | 10 |
| | T-2 w 37° C./75% r.h. | 5.466 | 43 |
| | T-stir | 58.755 | 61 |
| | T-unstir | 318 | 33 |
| | Placebo | 77 | 3 |
| FB6 | T0 | 238 | 13 |
| | T-FT | 69 | 3 |
| | T-1 w 25° C./60% r.h. | 161 | 13 |
| | T-1 w 37° C./75% r.h. | 6.064 | 75 |
| | T-2 w 25° C./60% r.h. | 298 | 16 |
| | T-2 w 37° C./75% r.h. | 3.628 | 121 |
| | T-stir | 55.619 | 34 |
| | T-unstir | 192 | 20 |
| | Placebo | 74 | 2 |
| FB7 | T0 | 100 | 3 |
| | T-FT | 187 | 10 |
| | T-1 w 25° C./60% r.h. | 1.337 | 51 |
| | T-1 w 37° C./75% r.h. | 7.652 | 165 |
| | T-2 w 25° C./60% r.h. | 205 | 8 |
| | T-2 w 37° C./75% r.h. | 13.235 | 959 |
| | T-stir | 6.392 | 36 |
| | T-unstir | 298 | 11 |
| | Placebo | 134 | 10 |
| FB8 | T0 | 169 | 23 |
| | T-FT | 410 | 18 |
| | T-1 w 25° C./60% r.h. | 77 | 8 |
| | T-1 w 37° C./75% r.h. | 5.042 | 547 |
| | T-2 w 25° C./60% r.h. | 339 | 13 |
| | T-2 w 37° C./75% r.h. | 6.013 | 326 |
| | T-stir | 8.681 | 43 |
| | T-unstir | 274 | 36 |
| | Placebo | 18 | 2 |

TABLE 16
Values of SvPs ≥2 μm or ≥10 μm following different stress
conditions, such as freeze-thaw (T-FT), mechanical stress (T-mech),
storage for one month at a temperature of −80° C, −20° C., or 25° C.,
or after temperature cycling from −20° C. to −80° C. or from
2-8° C. to −80° C.

| | | Particle concentration [#/ml] | |
|---|---|---|---|
| Formulation | Condition | ≥2 μm | ≥10 μm |
| FB2A | T0 | 56 | 7 |
| | T-FT | 577 | 15 |
| | T-mech | 16.044 | 33 |
| | T-1 m −80° C. | 252 | 3 |
| | T-1 m −20° C. | 226 | 7 |
| | T-1 m 25° C. | 2.407 | 93 |
| | T-cyc −20° C./−80° C. | 5.328 | 26 |
| | T-cyc 2-8° C./−80° C. | 552 | 16 |
| | CV [%] | 173 | |
| FB11 | T0 | 121 | 15 |
| | T-FT | 537 | 26 |
| | T-mech | 32.743 | 33 |
| | T-1 m −80° C. | 349 | 2 |
| | T-1 m −20° C. | 269 | 5 |
| | T-1 m 25° C. | 2.412 | 110 |
| | T-cyc −20° C./−80° C. | 3.080 | 15 |
| | T-cyc 2-8° C./−80° C. | 3.046 | 118 |
| | CV [%] | 210 | |
| FB12 | T0 | 238 | 16 |
| | T-FT | 393 | 25 |
| | T-mech | 41.915 | 146 |
| | T-1 m −80° C. | 277 | 3 |
| | T-1 m −20° C. | 179 | 8 |
| | T-1 m 25° C. | 4.345 | 270 |
| | T-cyc −20° C./−80° C. | 3.577 | 5 |
| | T-cyc 2-8° C./−80° C. | 932 | 8 |
| | CV [%] | 222 | |
| FB13 | T0 | 175 | 5 |
| | T-FT | 413 | 28 |
| | T-mech | 29.872 | 74 |
| | T-1 m −80° C. | 342 | 8 |
| | T-1 m −20° C. | 303 | 3 |
| | T-1 m 25° C. | 2.328 | 34 |
| | T-cyc −20° C./−80° C. | 3.470 | 20 |
| | T-cyc 2-8° C./−80° C. | 352 | 5 |
| | CV [%] | 220 | |
| FB14 | T0 | 162 | 11 |
| | T-FT | 383 | 13 |
| | T-mech | 21.169 | 41 |
| | T-1 m −80° C. | 187 | 7 |
| | T-1 m −20° C. | 257 | 8 |
| | T-1 m 25° C. | 8.281 | 780 |
| | T-cyc −20° C./−80° C. | 5.240 | 16 |
| | T-cyc 2-8° C./−80° C. | 1.060 | 7 |
| | CV [%] | 160 | |

In a first screening study it was shown that poloxamer 188 at a concentration of 5 mg/mL reduces the subvisible particle level of samples directly after preparation as well as after freeze-thaw, hold time (25° C. and 37° C. for 1 and 2 weeks) and stir stress (Table 15). In addition, in placebo solutions containing rHA, but no poloxamer 188, high number of subvisible particles were observed after 2 weeks at a temperature of 25° C., whereas a low number of subvisible particles were observed in the same formulation containing 5 mg/mL poloxamer 188. Interestingly, stress induced particles in rHA containing formulations could be effectively prevented by the addition of poloxamer 188.

The presence of rHA was stabilizing the virus solution upon storage at a temperature of 25° C. as indicated by TCID₅₀ (FIG. 20). A similar stabilizing effect of rHA was observed in WP4A, where it was stabilizing the virus upon storage at a temperature of 25° C. for one month, as indicated by TCID₅₀ analysis (data not shown).

A reduction of the arginine concentration from 150 mM to 50 mM (in the absence of rHA) had a negative impact on mechanical stress and hold time (1 months at 25° C.) stability.

The stabilizing effect of poloxamer 188 as already observed before, was examined for two different poloxamer 188 concentrations of 2.5 mg/ml and 5 mg/mL. The sample containing the higher concentration of 5 mg/mL poloxamer 188 showed a lower concentration of SvPs≥2 μm after mechanical stress. This sample also showed lower concentrations of particles≥2 μm and ≥10 μm after cycling between a temperature of 2-8° C. and −80° C.

The impact of pH on the stability of the virus in the presence of rHA and poloxamer 188 was examined in a further work package. The following pH values were examined: pH 6.5, pH 7.0, pH 7.5, and pH 7.8. Virus solutions of lower pH (i.e., pH 6.5 and pH 7.0) showed higher levels of SvPs after storage for 1 month at a temperature of 25° C. (Table 16). Furthermore, these samples also showed a slight loss in virus particles by NTA after mechanical stress and freeze-thaw cycling. A decrease in TCID₅₀ was observed in all samples stored for 1 month at a temperature of 25° C.

This decrease was most pronounced for the formulation candidates with the highest pH (i.e., pH 7.8) and with the lowest pH (i.e., pH 6.5). Further experiments with a pH 7.0 and 7.3 confirmed stable DP quality with pH 7.3 and pH 7.5.

Based on the results described above prior to the rHA and poloxamer 188 spiking step, a preferred virus solution consists of 10 mM Tris, 150 mM L-arginine, 200 mM trehalose at pH 7.5 (adjusted with phosphoric acid). After spiking rHA and poloxamer 188 to a final concentration of 5 mg/mL each, the formulation has the following composition: 9.75 mM Tris, 146 mM L-arginine, 195 mM trehalose, 5 mg/ml rHA, 5 mg/mL poloxamer 188, pH 7.5 (adjusted with ortho-phosphoric acid).

### Example 9

### Formulation Study, Testing Formulations by Using a “Design of Experiments” Approach

### Readout:

Formulations were tested in a stability study. All formulations containing L-Arginine, poloxamer 188 and recombinant human albumin (rHA) were stable over a wide range of concentrations. Trehalose was not acting as a stabilizer but was acting as an osmolyte. The formulations without poloxamer 188 were not as stable as formulations containing poloxamer 188.

TABLE 17
Formulations tested in stability study

| | | | L- | | Poloxamer | |
|---|---|---|---|---|---|---|
| | | Tris | Arginine | Trehalose | 188 | rHA |
| Formulation | pH¹ | [mM] | [mM] | [mM] | [mg/ml] | [mg/ml] |
| FB01 | 7.5 | 10 | 150 | 100 | 0.0 | 5 |
| FB02 | 7.5 | 10 | 150 | 200 | 5.0 | 5 |
| FB03 | 7.5 | 10 | 100 | 100 | 0.0 | 2 |
| FB04 | 7.5 | 10 | 100 | 0 | 0.0 | 10 |
| FB05 | 7.5 | 10 | 300 | 200 | 5.0 | 10 |
| FB06 | 7.5 | 10 | 150 | 0 | 2.5 | 10 |
| FB07 | 7.5 | 10 | 300 | 100 | 0.0 | 10 |
| FB08 | 7.5 | 10 | 300 | 200 | 2.5 | 5 |
| FB09 | 7.5 | 10 | 150 | 200 | 0.0 | 10 |
| FB10 | 7.5 | 10 | 100 | 0 | 5.0 | 5 |
| FB11 | 7.5 | 10 | 100 | 200 | 2.5 | 10 |
| FB12² | 7.5 | 10 | 150 | 100 | 2.5 | 5 |
| FB13 | 7.5 | 10 | 150 | 100 | 5.0 | 10 |
| FB14 | 7.5 | 10 | 150 | 0 | 2.5 | 2 |
| FB15 | 7.5 | 10 | 300 | 100 | 5.0 | 2 |
| FB16² | 7.5 | 10 | 150 | 100 | 2.5 | 5 |
| FB17 | 7.5 | 10 | 300 | 200 | 0.0 | 2 |
| FB18 | 7.5 | 10 | 300 | 0 | 0.0 | 5 |
| FB19 | 7.5 | 10 | 300 | 0 | 5.0 | 10 |
| FB20 | 7.5 | 10 | 100 | 200 | 5.0 | 2 |

¹pH was adjusted with ortho-phosphoric acid.
²FB12 and FB16 have an identical composition.

1. A pharmaceutical composition comprising an enveloped virus, AND a protein agent and/or a poly(ethylene oxide)/poly(propylene oxide) block copolymer.

2. The pharmaceutical composition of claim 1, wherein the poly(ethylene oxide) and poly(propylene oxide) block copolymer is a poloxamer.

3. The pharmaceutical composition of claim 1, wherein the poly(ethylene oxide) and poly(propylene oxide) block copolymer is poloxamer 188.

4. The pharmaceutical composition of claim 1, wherein the protein agent is albumin or gelatin.

5. The pharmaceutical composition of claim 1, wherein the protein agent is human serum albumin or recombinant human albumin.

6. The pharmaceutical composition of claim 1, wherein the poly(ethylene oxide) and poly(propylene oxide) block copolymer is poloxamer 188, and the protein agent is human serum albumin or recombinant human albumin.

7. The pharmaceutical composition of claim 6, comprising poloxamer 188 in a concentration between 0.01-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.01-40 g/L, 0.01-30 g/L, 0.01-20 g/L, 0.01-10 g/L, 0.01-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

8. The pharmaceutical composition of claim 6, comprising human serum albumin or recombinant human albumin in a concentration between 0.05-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.05-40 g/L, 0.05-30 g/L, 0.05-20 g/L, 0.05-10 g/L, 0.05-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

9. The pharmaceutical composition according to claim 1, further comprising at least one of an amino acid, a buffer, or a sugar.

10. The pharmaceutical composition of claim 9, wherein the amino acid is selected from the group consisting of alanine, arginine, phenylalanine, glutamic acid, glycine, methionine, lysine, and glutamine, preferably arginine.

11. The pharmaceutical composition of claim 9, wherein the buffer is selected from the group consisting of acetate, citrate, histidine, succinate, HEPES, tartrate, phosphate, citrate/phosphate, lactate, and Tris, preferably Tris.

12. The pharmaceutical composition of claim 9, wherein the sugar is selected from the group consisting of dextrose, fructose, galactose, glucose, raffinose, trehalose, and sucrose, preferably trehalose.

13. The pharmaceutical composition of claim 1, wherein the composition further comprises one or more sugar alcohols.

14. The pharmaceutical composition of claim 13, wherein the one or more sugar alcohol is selected from the group consisting of: mannitol, sorbitol, xylitol, maltitol, maltitol symp, lactitol, inositol, glycerol erythritol, isomalt, and hydrogenated starch hydroxylate.

15. The pharmaceutical composition of claim 14, wherein the one or more sugar alcohol is mannitol and/or sorbitol, preferably a combination of mannitol and sorbitol.

16. The pharmaceutical composition of claim 9, wherein the buffer has a concentration between 1-100 mM, 1-90 mM, 1-80 mM, 1-70 mM, 1-60 mM, 1-50 mM, 1-40 mM, 1-30 mM, 1-20 mM, or 1-10 mM.

17. The pharmaceutical composition of claim 9, wherein the sugar has a concentration between 10-1000 mM, 10-900 mM, 10-800 mM, 10-700 mM, 10-600 mM, 10-500 mM, 10-400 mM, 10-300 mM, 10-200 mM, 20-1000 mM, 20-900 mM, 20-800 mM, 20-700 mM, 20-600 mM, 20-500 mM, 20-400 mM, 20-300 mM, 20-200 mM, 30-1000 mM, 30-900 mM, 30-800 mM, 30-700 mM, 30-600 mM, 30-500 mM, 30-400 mM, 30-300 mM, 30-200 mM, 40-1000 mM, 40-900 mM, 40-800 mM, 40-700 mM, 40-600 mM, 40-500 mM, 40-400 mM, 40-300 mM, 40-200 mM, 50-1000 mM, 50-900 mM, 50-800 mM, 50-700 mM, 50-600 mM, 50-500 mM, 50-400 mM, 50-300 mM, or 50-200 mM.

18. The pharmaceutical composition of claim 1, wherein the composition is substantially free of chloride, preferably substantially free of NaCl.

19. The pharmaceutical composition of claim 1, wherein the pH of the composition is between 5 to 9, or between 6 to 9, or between 6.5 to 8.5, or between 6.5 to 8.0, preferably between 7.0 and 8.0.

20. The pharmaceutical composition of claim 19, wherein the pH of the composition is adjusted with phosphoric acid or sodium phosphate.

21. The pharmaceutical composition according to claim 1, comprising:
i) an enveloped virus,
ii) at least one of a buffer, an amino acid, or a sugar, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

22. The pharmaceutical composition according to claim 1, comprising:
i) an enveloped virus,
ii) an amino acid and a buffer, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

23. The pharmaceutical composition according to claim 1, comprising:
i) an enveloped virus,
ii) an amino acid and a sugar, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

24. The pharmaceutical composition according to claim 1, comprising:
i) an enveloped virus,
ii) an amino acid and a buffer and a sugar, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

25. The pharmaceutical composition according to claim 1, comprising:
i) an enveloped virus,
ii) a buffer and a sugar, AND
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

26. The pharmaceutical composition according to claim 1, comprising:
i) an enveloped virus,
ii) at least one of a buffer, an amino acid, or a sugar, wherein the buffer is selected from the group consisting of acetate, citrate, histidine, succinate, HEPES, tartrate, phosphate, citrate/phosphate, lactate, and Tris, preferably Tris, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

27. The pharmaceutical composition according to claim 1, comprising:
i) an enveloped virus,
ii) an amino acid and a buffer, wherein the buffer is selected from the group consisting of acetate, citrate, histidine, succinate, HEPES, tartrate, phosphate, citrate/phosphate, lactate, and Tris, preferably Tris, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

28. The pharmaceutical composition according to claim 1, comprising:
i) an enveloped virus,
ii) an amino acid and a buffer and a sugar, wherein the buffer is selected from the group consisting of acetate, citrate, histidine, succinate, HEPES, tartrate, phosphate, citrate/phosphate, lactate, and Tris, preferably Tris, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

29. The pharmaceutical composition according to claim 1, comprising:
i) an enveloped virus,
ii) a buffer and a sugar, wherein the buffer is selected from the group consisting of acetate, citrate, histidine, succinate, HEPES, tartrate, phosphate, citrate/phosphate, lactate, and Tris, preferably Tris, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

30. The pharmaceutical composition according to claim 1, comprising:
i) an enveloped virus,
ii) at least one of a buffer, an amino acid, or a sugar, wherein the sugar is selected from the group consisting of dextrose, fructose, galactose, glucose, raffinose, trehalose, and sucrose, preferably trehalose, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

31. The pharmaceutical composition according to claim 1, comprising:
i) an enveloped virus,
ii) an amino acid and a buffer, wherein the amino acid is selected from the group consisting of alanine, arginine, phenylalanine, glutamic acid, glycine, methionine, lysine, and glutamine, preferably arginine, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

32. The pharmaceutical composition according to claim 1, comprising:
i) an enveloped virus,
ii) an amino acid and a sugar, wherein the sugar is selected from the group consisting of dextrose, fructose, galactose, glucose, raffinose, trehalose, and sucrose, preferably trehalose, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

33. The pharmaceutical composition according to claim 1, comprising:
i) an enveloped virus,
ii) an amino acid and a buffer and a sugar, wherein the sugar is selected from the group consisting of dextrose, fructose, galactose, glucose, raffinose, trehalose, and sucrose, preferably trehalose, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

34. The pharmaceutical composition according to claim 1, comprising:
i) an enveloped virus,
ii) a buffer and a sugar, wherein the sugar is selected from the group consisting of dextrose, fructose, galactose, glucose, raffinose, trehalose, and sucrose, preferably trehalose, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

35. The pharmaceutical composition according to claim 1, comprising:
i) an enveloped virus,
ii) at least one of a buffer, an amino acid, or a sugar, wherein the amino acid is selected from the group consisting of alanine, arginine, phenylalanine, glutamic acid, glycine, methionine, lysine, and glutamine, preferably arginine, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

36. The pharmaceutical composition according to claim 1, comprising:
i) an enveloped virus,
ii) an amino acid and a sugar, wherein the amino acid is selected from the group consisting of alanine, arginine, phenylalanine, glutamic acid, glycine, methionine, lysine, and glutamine, preferably arginine, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

37. The pharmaceutical composition according to claim 1, comprising:
i) an enveloped virus,
ii) an amino acid and a buffer and a sugar, wherein the amino acid is selected from the group consisting of alanine, arginine, phenylalanine, glutamic acid, glycine, methionine, lysine, and glutamine, preferably arginine, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

38. The pharmaceutical composition according to claim 1, comprising:
i) an enveloped virus,
ii) at least one of a buffer, an amino acid or a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably arginine; wherein the sugar is selected from the group consisting of dextrose, fructose, galactose, glucose, raffinose, trehalose, and sucrose, preferably trehalose, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

39. The pharmaceutical composition according to claim 1, comprising:
i) an enveloped virus,
ii) an amino acid and a buffer, wherein the buffer is selected from the group consisting of acetate, citrate, histidine, succinate, HEPES, tartrate, phosphate, citrate/phosphate, lactate, and Tris, preferably Tris; wherein the amino acid is selected from the group consisting of alanine, arginine, phenylalanine, glutamic acid, glycine, methionine, lysine, and glutamine, preferably arginine, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

40. The pharmaceutical composition according to claim 1, comprising:
i) an enveloped virus,
ii) an amino acid and a sugar, wherein the amino acid is selected from the group consisting of alanine, arginine, phenylalanine, glutamic acid, glycine, methionine, lysine, and glutamine, preferably arginine; wherein the sugar is selected from the group consisting of dextrose, fructose, galactose, glucose, raffinose, trehalose, and sucrose, preferably trehalose, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

41. The pharmaceutical composition according to claim 1, comprising:
i) an enveloped virus,
ii) an amino acid and a buffer and a sugar, wherein the buffer is selected from the group consisting of acetate, citrate, histidine, succinate, HEPES, tartrate, phosphate, citrate/phosphate, lactate and Tris, preferably Tris; wherein the amino acid is selected from the group consisting of alanine, arginine, phenylalanine, glutamic acid, glycine, methionine, lysine, and glutamine, preferably arginine; wherein the sugar is selected from the group consisting of dextrose, fructose, galactose, glucose, raffinose, trehalose, and sucrose, preferably trehalose, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

42. The pharmaceutical composition according to claim 1, comprising:
i) an enveloped virus,
ii) a buffer and a sugar, wherein the buffer is selected from the group consisting of acetate, citrate, histidine, succinate, HEPES, tartrate, phosphate, citrate/phosphate, lactate, and Tris, preferably Tris; wherein the amino acid is selected from the group consisting of alanine, arginine, phenylalanine, glutamic acid, glycine, methionine, lysine, and glutamine, preferably arginine, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

43. The pharmaceutical composition according to claim 1, comprising:
i) an enveloped virus,
ii) a buffer, wherein the buffer is Tris, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

44. The pharmaceutical composition according to claim 1, comprising:
i) an enveloped virus,
ii) an amino acid, wherein the amino acid is arginine, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

45. The pharmaceutical composition according to claim 1, comprising:
i) an enveloped virus,
ii) a sugar, wherein the sugar is trehalose or sucrose, and
iii) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

46. The pharmaceutical composition according to claim 1, comprising:
i) an enveloped virus,
ii) an amino acid, wherein the amino acid is arginine,
iii) a sugar, wherein the sugar is trehalose or sucrose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

47. The pharmaceutical composition according to claim 1, comprising:
i) an enveloped virus,
ii) a buffer, wherein the buffer is Tris,
iii) a sugar, wherein the sugar is trehalose or sucrose, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

48. The pharmaceutical composition according to claim 1, comprising:
i) an enveloped virus,
ii) a buffer, wherein the buffer is Tris,
iii) an amino acid, wherein the amino acid is arginine, and
iv) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

49. The pharmaceutical composition according to claim 1, comprising:
i) an enveloped virus,
ii) a buffer, wherein the buffer is Tris
iii) an amino acid, wherein the amino acid is arginine,
iv) a sugar, wherein the sugar is trehalose or sucrose, and
v) a protein agent and/or a poly(ethylene oxide)/poly(propylene oxide) block copolymer,
wherein the protein agent is selected from human serum albumin or recombinant human albumin and the poly(ethylene oxide)/poly(propylene oxide) block copolymer is poloxamer 188.

50. The pharmaceutical composition of claim 21, wherein the buffer has a concentration between 1-100 mM, 1-90 mM, 1-80 mM, 1-70 mM, 1-60 mM, 1-50 mM, 1-40 mM, 1-30 mM, 1-20 mM, or 1-10 mM.

51. The pharmaceutical composition of claim 21, wherein the sugar has a concentration between 10-1000 mM, 10-900 mM, 10-800 mM, 10-700 mM, 10-600 mM, 10-500 mM, 10-400 mM, 10-300 mM, 10-200 mM, 20-1000 mM, 20-900 mM, 20-800 mM, 20-700 mM, 20-600 mM, 20-500 mM, 20-400 mM, 20-300 mM, 20-200 mM, 30-1000 mM, 30-900 mM, 30-800 mM, 30-700 mM, 30-600 mM, 30-500 mM, 30-400 mM, 30-300 mM, 30-200 mM, 40-1000 mM, 40-900 mM, 40-800 mM, 40-700 mM, 40-600 mM, 40-500 mM, 40-400 mM, 40-300 mM, 40-200 mM, 50-1000 mM, 50-900 mM, 50-800 mM, 50-700 mM, 50-600 mM, 50-500 mM, 50-400 mM, 50-300 mM, or 50-200 mM.

52. The pharmaceutical composition of claim 21, comprising poloxamer 188 in a concentration between 0.01-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.01-40 g/L, 0.01-30 g/L, 0.01-20 g/L, 0.01-10 g/L, 0.01-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

53. The pharmaceutical composition of claim 21, comprising human serum albumin or recombinant human albumin in a concentration between 0.05-50 g/L, 0.1-50 g/L, 0.2-50 g/L, 0.3-50 g/L, 0.4-50 g/L, 0.5-50 g/L, 1-50 g/L, 2-50 g/L, 3-50 g/L, 4-50 g/L, 5-50 g/L, 0.05-40 g/L, 0.05-30 g/L, 0.05-20 g/L, 0.05-10 g/L, 0.05-5 g/L, 0.1-40 g/L, 0.1-30 g/L, 0.1-20 g/L, 0.1-10 g/L, 0.1-5 g/L, 0.2-40 g/L, 0.2-30 g/L, 0.2-20 g/L, 0.2-10 g/L, 0.2-5 g/L, 0.3-40 g/L, 0.3-30 g/L, 0.3-20 g/L, 0.3-10 g/L, 0.3-5 g/L, 0.4-40 g/L, 0.4-30 g/L, 0.4-20 g/L, 0.4-10 g/L, 0.4-5 g/L, 0.5-40 g/L, 0.5-30 g/L, 0.5-20 g/L, 0.5-10 g/L, 0.5-5 g/L, 1-40 g/L, 1-30 g/L, 1-20 g/L, 1-10 g/L, 1-5 g/L, 2-40 g/L, 2-30 g/L, 2-20 g/L, 2-10 g/L, 3-40 g/L, 3-30 g/L, 3-20 g/L, 3-10 g/L, 4-40 g/L, 4-30 g/L, 4-20 g/L, 4-10 g/L, 5-40 g/L, 5-30 g/L, 5-20 g/L, or 5-10 g/L.

54. The pharmaceutical composition of claim 1, comprising:
i) an enveloped virus,
ii) about 1-100 mM Tris,
iii) about 10-500 mM Arginine,
iv) about 10-500 mM Trehalose
v) about 0.1-5 mg/ml Poloxamer 188,
vi) about 0.5-10 mg/ml recombinant Human albumin, and
vii) a pH of about 6 to 8.

55. The pharmaceutical composition of claim 54, comprising:
i) an enveloped virus
ii) about 10 mM Tris
iii) about 150 mM Arginine
iv) about 100 mM Trehalose
v) about 0.5 mg/ml Poloxamer 188,
vi) about 2 mg/ml recombinant Human albumin, and
vii) a pH of about 7.5.

56. The pharmaceutical composition of claim 54, wherein the pH of the composition is adjusted with phosphoric acid or sodium phosphate.

57. The pharmaceutical composition according to claim 1, wherein the enveloped virus is a rhabdoviridae, preferably a vesiculovirus or vesicular stomatitis virus (VSV).

58. The pharmaceutical composition according to claim 57, wherein the enveloped virus is a recombinant vesicular stomatitis virus (VSV), wherein the gene coding for the glycoprotein G of the vesicular stomatitis virus is replaced by the gene coding for the glycoprotein GP of LCMV, and/or the glycoprotein G is replaced by the glycoprotein GP of LCMV.

59. The pharmaceutical composition according to claim 57, wherein the pharmaceutical composition comprises the enveloped virus, preferably the vesiculovirus or vesicular stomatitis virus (VSV), in a concentration of at least 1×10⁵ TCID₅₀/mL, at least 1×10⁶ TCID₅₀/mL, at least 1×10⁷ TCID₅₀/mL, at least 1×10⁸ TCID₅₀/mL, at least 1×10⁹ TCID₅₀/mL, or at least 1×10⁹ TCID₅₀/mL.

60. The pharmaceutical composition according to claim 57, wherein the pharmaceutical composition comprises the enveloped virus, preferably the vesiculovirus or vesicular stomatitis virus (VSV), in a concentration range between 1×10⁵ TCID₅₀/mL to 1×10¹² TCID₅₀/mL, between 1×10⁶ TCID₅₀/mL to 1×10¹² TCID₅₀/mL, between 1×10⁷ TCID₅₀/mL to 1×10¹² TCID₅₀/mL, between 1×10⁸ TCID₅₀/mL to 1×10¹² TCID₅₀/mL, 1×10⁵ TCID₅₀/mL to 1×10¹¹ TCID₅₀/mL, 1×10⁵ TCID₅₀/mL to 1×10¹⁰ TCID₅₀/mL, or 1×10⁵ TCID₅₀/mL to 1×10⁹ TCID₅₀/mL.

61. A pharmaceutical composition according to claim 1, wherein the pharmaceutical composition is a liquid or frozen liquid pharmaceutical composition.

62. The liquid pharmaceutical composition according to claim 61, wherein the composition is frozen and stored at a temperature of about −80° C., −70° C., −60° C., −50° C., −40° C., −35° C., −30° C., −25° C., −20° C., −15° C., −10° C., or −5° C.

63. A product produced by lyophilizing the liquid pharmaceutical composition of claim 62.

64. A dry pharmaceutical composition produced by a method comprising removing water from a pharmaceutical composition according to claim 1.

65. The dry pharmaceutical composition of claim 64, wherein the pharmaceutical composition is frozen to obtain a pharmaceutical composition comprising ice prior to removing water.

66. The dry pharmaceutical composition of claim 65, wherein the method further comprises placing the liquid pharmaceutical composition in a vacuum under controlled temperatures and pressure to remove the water.

67. The dry pharmaceutical composition of claim 64, wherein the method is lyophilization.

68. The dry pharmaceutical composition of claim 64, comprising less than about (0.5%-5%) w/w water.

69. A pharmaceutical composition comprising water and the product of claim 61.


SEQUENCE LISTING (8 sequences)

SEQ ID NO: 1 - protein, 422 aa, Vesicular stomatitis virus
MSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 2 - protein, 265 aa, Vesicular stomatitis virus
MDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 3 - protein, 2109 aa, Vesicular stomatitis virus
MEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 4 - protein, 229 aa, Vesicular stomatitis virus
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 5 - protein, 498 aa, Lymphocytic choriomeningitis mammarenavirus
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 6 - RNA, 11169 nt, Vesicular stomatitis virus
  source 1..11169: mol_type = genomic RNA
tgcttctgtttgtttggtaataatagtaattttccgagtcctctttgaaattgtcattagttttacagacaatgtcagttctcttagtaactgttgtgtcagcatcaaggttttgaaggacgtttactcctaggtcaccttatgggccgtctaatgaagtcttttagtttcctctaaggagaaatgtagttatgatgtttttcaaacagtctagattctcctatacagatggttccggagtttaggcctttacatagttagtatgtacagttgtcgatgaacatacctcgtaatttcctgtaggccccattcaacctatttctaaccagttcaaagccttatttgtagccctttcgtcccctatgttagccttataaactggaacataggaactttcgggacctgccgcatgaaggtctacctcatagcctacgaaggtcttggtcgcgtctactgtttaccaacggaaacatagatgaaccgaatatgtctcacccgtcttgtgtttacggacttatgtcttttttcgagtacctacccgactgtttagttacgttttactagttacttgtcaaacttggagaacacggtcttccagcactgtaaaaactacacacccctttactgtcattaatgtgtttttaacagcgacgtcacctgtacaagaaggtgtacaagttttttgtacttacacggagcaagtctatgccttgataacaaaggtctaagtttctaacacgacgtaaccgttgtaaacctgtggagacgttttattggccttacagatgtcttctacattgctggacctagaacttggctcttcaacgtctactttaccaggtttactacgaaggtccggttctttaactgttccggctaagtatgtacggaataaactagctgaaacctaacagaagattcagaggtataagaaggcagtttttgggacggaaggtgaagacccccgttaactgtcgagaagacgagtctaggtggtctcgttccttacgggctgtcggactactgtaactcatatgtagagaatgatgtcgtccaaacaacatgcgaatacgtcatcctaggagacggctgaaccgtgttgtcaaaacacaacctctattgtttatgtgaggtctactatcatggcctcctaactgctgattacgtggcggtgttccgtctctacaccagcttaccgagcctaccaaacttctagttttgtcttttggctgaggactatactacgtcatacgcttttctcgtcagtacagtgacgttccggattctctcttctgttaaccgttcatacgattcagtcttaaactgtttactgggatattaagagtctagtggataatatataatacgatgtatactttttttgattgtctatagtacctattagagtgttttcaagcactcatagagttcaggataagagcagacctagtccgccatcctctctatctactctagcttcgtgttgctcgacttttcaggttaatactcaacaaggttctcctacctcaccttctcgtatgattcgggagaataaaagtccgtcgtctactaagactgtgtcttagacttggtctttaacttctgttagttccgaacatacgtggtctaggtcttcgactcgttcaacttccgaaatatgtccccggaaatctactgatacgtctactccttcacctacaacataaatgaagcctgacctttgtcggactcgaacttagactgctcgtacctttctggaatgccaactgtagcggtctcccaaattcacctctcgtctttagggtcaccgaaagctgctaatttcgtcagcacgtttcacggtttatgaccttagaccgtctcacgtgtaaacttcgtagccctcttccccagtaatacttcctcgcggtctattgaggcctacatatattccagtgaggtcactacttgtgtgtaggcagggttagtcttcgtcatagtctacaaaccagagagagtttctgtaggtactgaaaggttgggttctttcgttcagaagtcggagagtggtataggaacctacttaacaagagtagatctcctctcaagtagagacagcctccactgcctgcttacagagtatttctccggtaggacgagccggactctatgtttttcaacatgttagtccgctctcagtttataagagacatctgatactttttttcattgtctatagtgctagattcacaatagggttaggtaagtagtactcaaggaatttcttctaagagccagacttcccctttccattctttagattctttaatccctagcgtggtgggggaatacttctcctgtgatcgtacctcatacgaggctcgcgaggttaactgtttaggataaaacctcaactgctctacctgtggatactaggcttagttaattctatactctttaagaagaaatgtcacttttactgccaatctagattagcaggcaagtcttgtatgagtctacaccgtcggcgacatagggtaaccctagtgtacatgtagccttaccgtccctttgcagggaagatgttttagaaccgaaaaaacccaagaagattagatttccggtgaggtcgccataaccgtctagttccagttggtctcatagtgcgagtgacgcttccgtcccgaataaacggtgtatcctaccccttctggggagggtacgagttacatggtctcgtgaagtcttctggtaagttatatccagaaatgttcccttgctaactcgagtgttactggtagatgctactactcagtgaccttcgtcgaggatactagaccctagtaaagttaagaaggtttaaaagactaaagtctctcttccggaattacaaaccggactaacagctctttttccgtagacctcgcacccaggacctgagatagccggtgaagtttactcgatcagattgaagatcgaagacttgttaggggccaaatgagtcagaggggattaaggtcggagagcttgttgattataggacagaaaagatagggatactttttttgattgtctctagctagacaaatgcgcagtgcctagggggcccgacgtccttaagcggtggtacccggtctagcactggtacaagctccgggacggggtgtagtagctgctccactagttgtagcactagtagcacgagtagtagtagtggtcgtagttccggcacatgttgaagcggtggacgccgtaggaccgggaccactcgaaggacaaggaccggccgtcttcgacgccgtacatgccggacttaccggggctatagatgttcccgcacatggtcaagttctcgcacctcaagctgtactcggtggacttggagtggtacgggttgcggacgtcgcggttgttatcggtggtgatgtagtcgtacccgtcgtcgccggacctcaactggaagtggttgctgtcgtaggacttggtgttgaagacgttggagtggtcgcggaagttgttcttttggaagctggtgtgggagtactcgtagcactcgtcggacgtggactcgtagtctccgttgtcgttggtgttccggcactcgacgctgaagttgttgccgtagtggtaggtcatgttggactcgaagtcgctaggagtctcgcggtagtcggtcacgtcttggaagtctccgtctcacgacctgtacaagtcttggcggaagccgccgttcatgtactcttcgccgaccccgacccggccgtcgctgccgttctggtggaccacgtcggtctggtcgatggtcatggagtagtaggtcttgtcttggaccctcttggtgacgtctatgcggcctggaaagccgtactcgtcttaggacaagcgggtcctcttttggttcaaggagtggtcctctgaccggccgtggaagtggacctgggactcgctgtcgtcgccgcacctcttgggaccgccgatgacggagtggttcacctactaggaccggcggctcgacttcacgaagccgttgtggcggcaccggttcacgttgcacttggtgctgctcctcaagacgctgtacgactctgagtagctgatgttgttccggcgggactcgttcaagttcgtcctgcacctctcgcgggacgtgcacaagttctggtggcacttgtcggagtagtcgctggtcgacgagtactctttggtggactctctggagtacccgcacgggatgacgttgatgtcgttcaagaccatagacctcgtgcggttctggccgctctggtcgcacgggttcacgaccgaccactggttaccgtcgatggacttgctctgggtgaagtcgctggtctagctcgtccttcggctgttgtactagtggctctacgactccttcctgatgtagttctctgtcccgtcgtggggggaccgggagtacctagacgagtacaagtcgtggtcgcggatggagtagtcgtagaaggacgtggaccacttctaggggtgggtgtctgtgtagttcccgccgtcgacggggttcggggtgtctgagtggttgttcccgtagacgtcgacgccgcggaagttccacgggccgcacttttggtagaccttctcctctattcgccggcgatgctggagctcttaattaacgatcggtctaagaagtacaaacctggtttagttgaacactatggtacgagtttctccggagttaatataaactcaaaaattaaaaatactttttttgattgtcgttagtaccttcaggtgctaaaactctggctgctcaagttactaaagttacttctactgatacggtgttctcttaaggacttagggctactcgcgtactgcatgaacttagtacgactaatgttggacttaagaggagattaatcactactataactgttaaattagtcctttaagttaagagaaggttaagggagctacaccctatcattcttgaccctacctcaagaactctacaattgcagtacagttcggttagggtagggttgtagagtctacgtatttacctacccttcaaccaattacagactattagtactacggtcagttcccatatcaaaaaatgtacttcacctgtttctccgtctttattgtaaactgcaccacctctggaagtaggcgccgaccccgttgtttggttaacttatgtagtttttcctttctacctgactgagtaagttttaagagcgaataaacacagttttcaaaaacctgaatgtgttcaactgtaattagaatttacgacagagactccaccttaacgagttgaaccgctcctgaaagtttccgtttcagtcttcttcaagagtaccttgcttgtatacgtcctaatcccaagggtcgaacccaggatgaaaataaagtcttcctacccgaatgaagttctttgaactataagattacctggctttgaaagacaattaccagtttctacactaatatccctcctacgtttgccacgataggtaccatacatcttatctgttggacaagagtctcgttctgtagaagagggaagatttatagatgtcttaacctctattttaacacctctccgtccctttaaaaagaatactgaactaattttaccaccttggctatacgttgaacttcgactactttaatcgttctcttagttccggaaatcagggtgttaagggagtaaaacttttagtatagttctgaagacaactacttccccgtttttaactggctccatattctaaggaggtactagtctattactcacacttttgtcacctagagtgtgaccactaaatacctagcaagtctgtaaccccagtaggaaaatatctaataatgtgacctgatctttttaatgtaagggttcattggtacttctttctataactacacagtatacgttttcgtgaacgttcactaaatcgagcctaacaagataaagttgtcaagttactagtatttttcaccaagcacttacctctgaacgagggagtactagtagggaaattttcagtacaatttcttttatgtaccgggtgtcgacgagttcaagttctaaaacctctatttaccgtacttgaaggcgactaatttacaaaactttatgggctgaatgatctgggtagctattatatgagactgttttcagtaagttacttatccagtctccacaactttgtacaggcttacttaggcttgtgaggatagggatcatttttccacaacgtctgatacaacctgtgtttccgatggttaacctttcttaaagaatttctctaactactcttcccgaatctactactactagattaataaccagaatttcctttcctctcccttgacttcaaccgtccatctaaaaagagggattacagaacctttaacgctcttatgaaacattaatggcttataaactatttctgagtaaagcagggatacaaatttccggactgttaccgcctgctagattgacgtcagtaatttttctacaatctaaggagtaggccggttcctaacttcagtatactccgttaaacgtatcggttagtgtaactaatgctttttaccttattggtggtttccttcaatagtttgccgggtcacaaggctcaatacccggtcaagaatccaataggtaggaattagctctcttgagtacttaaaaaactcttttcagaatatatgatgttaccttctggtctgaactacgcacaagtgttgttgtgtgactagttaagttggagggttgctcaaacaaccgttcctgttctcccacctgaccttccagatgccgtttttcctacctcataggagttagatgaccaataagtttctctccgattttagtctttgtgacgacagtttcagaaccgtgttccactattagttcaataaacgtgtgtcatattttgcttctttagctctttgcaacatcttaatgtcccacgagagttagtttaccaaagattattactcttttaatactgacgttagttttatccctgtcccttcaatcctgaaaactatttactgctactctgatacgttagacgtctaatgaacttaatacctttttatggctaaaaggcacctcactaatctcccaatctctggttctctaccagtgctcactgaacacagtggttactggtttatgggtgaacacgattatattactcgagtcaaaggtgtttacgagagtggcatcgagtaaaacgactcttgggttagttacggtactatgtcatgttaataaaaccctgtaaacgatctgagaacaactactacgtactaggacgagaagcagttagtaacatacttcaagttctattctatggcccgaacgtgtcaagatgaaagtttatgcggtacaacataaacctgggaaggtaacctcctcacagcccgtacagaaacaggtccaaaaactaatctcggaagggtctagggcattgtctttcagagagtaagacctctaagtaggtacatgtacgagcttcactcgtagacttcctctactcacgtcataaacctttggggctctatcggttcaaagcttattgagtgtatctgttcgatcatcttctaggttggagagacttgtagcgatacccttactcaggtcgcttgaacaatttctgactccaattttttacgaattagcttagttctgtttggtagtccttggtccactaattcctacgttggtatataaacatagtacttctcctagccgagtcttcaaagaataccagttatttaggagacaagggatctaaaaattcacttaagtttagtccgtgaaaaaaccctcagcgtctgcccgagtagtcagataaagttttaagagcatgataagccttgaggaaattctttttcatagtatcccttaacctactaaactaacactcctcactccataggagaaactgtgtaaatccctttgaagtaaactcttcccctagtacattttacacctgtacaagtcgatgagtacgactgtgtaattctatgtttaggaccccggcatgtcaataaccctgttgacatggggtaggtaatctttacaacccaggtgttgtagcttttctctgaggaacacgtggtacattgtgtagtcccaagttaatacaaagacacgtaacaggtctgccctaggtactgcagaaatcaagtgcccctggtaacggacgaatagatcccagattttgtagacttagatgtagataaaacgtcggaaccctttccctttcgtttcagggtgactaattttctcgatgtgcagaatctctacgatagagaaccaaacaacttgggctgagatttgatcgttactgatatgaaagattgtaggtgagaaattgtccgcttcttacctggttttccgtcgtacccaagttttcttgtcccagacgggaagtatccaaaagctgtagagcctactcggtaccacccaagcgtagagtctcgtgacgtcgtaactggtccaactaccgttgatgtctgtggtactccctagaccctctagtcttaaagctgaaaaataaggttcgttgcaacgagatacgagtttaatggtggtgacaacgttctctgcctacctagtggtcaacatgtctagtaatagtataacggacattcaggacaaactctgggtatcttctctagtgggacctgagttcatacctgatgtgcgggggtctacatagggtacacgacttctgtacctccttaccccttccaagcacccctgttctctattttgtctagataggaaatcttcccttaaccttcttaaatcgtggacgactcgttaggatagttcagccgtctacatatccaaaagatatacctctgaaccgcatatcttttagatgagtacggctcctgtcaagagataaaggagatagatatgttccagcataatctccagctccaaagaattttcccaacgatctgcctaattactctcgttcaacgacggttcattatgtggcctcttcagaccgagtaaacttctccggccggttgcgtcacatgcctccaaactaaatgaactaactatttaactcacatagtggaggtaaggaaagagaatgatctagtcctggataatctctgcttaatctttgctaaggggtgttctagggttggaggataggctgttcgttggcactatacccccactaacagtctttaatgaagtttatggttacggcagattaacttttcccttttatgtctagtgtaataagtgttaataccaataagagtctacagaataggtatctgaagtaacctggtaagagataaaggtggtgggagaacgtttaggatatgttcggtaaaaatagaccctttctattcttactcaactctctcgaccgtttagaaagaagtaacgattctagtcctctccccacccttctgtatgtacactttaagaagtggttcctgtataataacacaggtctcctttagtctgtacgaacgttcaagccctaacgattcctattattatttctgtactcgatagggggaaccccttcccttaggtctccctgttaatgttgttagggacaaataatatgctggtggggaatgggtttctacgatctctacggaggttcttaggttttaggggacgacaggccttagtccaacccggttaatggttgaccgcgagtaatattttaagcctcatataatgtaccttacccttaggtaatgtccctgaagaactcaacacctctgccgaggcctccctactgacgacgtaatgatgctcttttacacgtatcgtctccttataagttatcagacaatcttaatagtcccagtcagtacgctccgcggagaggactcggggggtcacgggatctttgaaatcctcctctatttagctctacacatttaccactttgtacaacccttataggtagactgaatacactgggttcctgaaccctgataaaggaggctgagtttcgtccgaaccccgaagtttaactaaattaacattacctataccttcaagccctaagaagatgatcggacttttaactctgcttacaatctttaatacacgtggcctaaaacctactcgttcctcaaaattagatgttctgaataccttgtatataaacactctcgcttttcttacgtcattgttaggaaccagggtacaagttctgccagctgaatcaagtttgtcttaaatcatcaagagtttgcagacttcatatataccatacatttccaaacttctttaattagctacttgggttagggctaaccagaaggtagttacttaggacctttttggacatgcgtaaggtcagtagtcttgtccttaaacggtctcgtttcttccaatcatgtatgaaatggaactgtccataagggagggttaagtaaggactaggaaaacatttgtaactctgatacgatgtttataagcctcatgggtgcccacacagagtacgccgacggaattttagtagactatctggacgtctaaataactggtaatcggaaaaaatataccgctaatatagcataatattgtagttagtatagtctcatcctggctatggaggcttggggggtagtctaccttaacgtgttttacacccctagcgatattgaccatattcgaaaaccgactcaaactacctctttctgtaaggtgatatagttgtcacaaatcgtcaataggtcgttagtaagggctaatccaccctccgacaaagtcattttcctcctatgttcgtcttcacctcatgatctccactacccgagggttttctatgggcttaaagtctgaggaaccggggttagcccttgacctagtctagagaccttaaccaggctttggttcaagcagatttaggtaagttactctagaacaagttagtcgatacagcatgtcacctattagtaaactttaccagtttaaacgcttctttgtgtccttactaacttacctagttatctgcttaaagttttcttctggccagatatgactacaacttctcactggatgtgctccttttgagaacctctctaattttttagtactcctctgaggtttgaaattcatactttttttgaaactaggaattctgggagaacaccaaaaataaaaaatagaccaaaacaccagaagca

SEQ ID NO: 7 - RNA, 11520 nt, Vesicular stomatitis virus
  source 1..11520: mol_type = genomic RNA
tgcttctgtttgtttggtaataatagtaattttccgagtcctctttgaaattgtcattagttttacagacaatgtcagttctcttagtaactgttgtgtcagcatcaaggttttgaaggacgtttactcctaggtcaccttatgggccgtctaatgaagtcttttagtttcctctaaggagaaatgtagttatgatgtttttcaaacagtctagattctcctatacagatggttccggagtttaggcctttacatagttagtatgtacagttgtcgatgaacatacctcgtaatttcctgtaggccccattcaacctatttctaaccagttcaaagccttatttgtagccctttcgtcccctatgttagccttataaactggaacataggaactttcgggacctgccgcatgaaggtctacctcatagcctacgaaggtcttggtcgcgtctactgtttaccaacggaaacatagatgaaccgaatatgtctcacccgtcttgtgtttacggacttatgtcttttttcgagtacctacccgactgtttagttacgttttactagttacttgtcaaacttggagaacacggtcttccagcactgtaaaaactacacacccctttactgtcattaatgtgtttttaacagcgacgtcacctgtacaagaaggtgtacaagttttttgtacttacacggagcaagtctatgccttgataacaaaggtctaagtttctaacacgacgtaaccgttgtaaacctgtggagacgttttattggccttacagatgtcttctacattgctggacctagaacttggctcttcaacgtctactttaccaggtttactacgaaggtccggttctttaactgttccggctaagtatgtacggaataaactagctgaaacctaacagaagattcagaggtataagaaggcagtttttgggacggaaggtgaagacccccgttaactgtcgagaagacgagtctaggtggtctcgttccttacgggctgtcggactactgtaactcatatgtagagaatgatgtcgtccaaacaacatgcgaatacgtcatcctaggagacggctgaaccgtgttgtcaaaacacaacctctattgtttatgtgaggtctactatcatggcctcctaactgctgattacgtggcggtgttccgtctctacaccagcttaccgagcctaccaaacttctagttttgtcttttggctgaggactatactacgtcatacgcttttctcgtcagtacagtgacgttccggattctctcttctgttaaccgttcatacgattcagtcttaaactgtttactgggatattaagagtctagtggataatatataatacgatgtatactttttttgattgtctatagtacctattagagtgttttcaagcactcatagagttcaggataagagcagacctagtccgccatcctctctatctactctagcttcgtgttgctcgacttttcaggttaatactcaacaaggttctcctacctcaccttctcgtatgattcgggagaataaaagtccgtcgtctactaagactgtgtcttagacttggtctttaacttctgttagttccgaacatacgtggtctaggtcttcgactcgttcaacttccgaaatatgtccccggaaatctactgatacgtctactccttcacctacaacataaatgaagcctgacctttgtcggactcgaacttagactgctcgtacctttctggaatgccaactgtagcggtctcccaaattcacctctcgtctttagggtcaccgaaagctgctaatttcgtcagcacgtttcacggtttatgaccttagaccgtctcacgtgtaaacttcgtagccctcttccccagtaatacttcctcgcggtctattgaggcctacatatattccagtgaggtcactacttgtgtgtaggcagggttagtcttcgtcatagtctacaaaccagagagagtttctgtaggtactgaaaggttgggttctttcgttcagaagtcggagagtggtataggaacctacttaacaagagtagatctcctctcaagtagagacagcctccactgcctgcttacagagtatttctccggtaggacgagccggactctatgtttttcaacatgttagtccgctctcagtttataagagacatctgatactttttttcattgtctatagtgctagattcacaatagggttaggtaagtagtactcaaggaatttcttctaagagccagacttcccctttccattctttagattctttaatccctagcgtggtgggggaatacttctcctgtgatcgtacctcatacgaggctcgcgaggttaactgtttaggataaaacctcaactgctctacctgtggatactaggcttagttaattctatactctttaagaagaaatgtcacttttactgccaatctagattagcaggcaagtcttgtatgagtctacaccgtcggcgacatagggtaaccctagtgtacatgtagccttaccgtccctttgcagggaagatgttttagaaccgaaaaaacccaagaagattagatttccggtgaggtcgccataaccgtctagttccagttggtctcatagtgcgagtgacgcttccgtcccgaataaacggtgtatcctaccccttctggggagggtacgagttacatggtctcgtgaagtcttctggtaagttatatccagaaatgttcccttgctaactcgagtgttactggtagatgctactactcagtgaccttcgtcgaggatactagaccctagtaaagttaagaaggtttaaaagactaaagtctctcttccggaattacaaaccggactaacagctctttttccgtagacctcgcacccaggacctgagatagccggtgaagtttactcgatcagattgaagatcgaagacttgttaggggccaaatgagtcagaggggattaaggtcggagagcttgttgattataggacagaaaagatagggatactttttttgattgtctctagctagacaaatgcgcagtgcctagggggcccgacgtccttaagcggtggtacccggtctagcactggtacaagctccgggacggggtgtagtagctgctccactagttgtagcactagtagcacgagtagtagtagtggtcgtagttccggcacatgttgaagcggtggacgccgtaggaccgggaccactcgaaggacaaggaccggccgtcttcgacgccgtacatgccggacttaccggggctatagatgttcccgcacatggtcaagttctcgcacctcaagctgtactcggtggacttggagtggtacgggttgcggacgtcgcggttgttatcggtggtgatgtagtcgtacccgtcgtcgccggacctcaactggaagtggttgctgtcgtaggacttggtgttgaagacgttggagtggtcgcggaagttgttcttttggaagctggtgtgggagtactcgtagcactcgtcggacgtggactcgtagtctccgttgtcgttggtgttccggcactcgacgctgaagttgttgccgtagtggtaggtcatgttggactcgaagtcgctaggagtctcgcggtagtcggtcacgtcttggaagtctccgtctcacgacctgtacaagtcttggcggaagccgccgttcatgtactcttcgccgaccccgacccggccgtcgctgccgttctggtggaccacgtcggtctggtcgatggtcatggagtagtaggtcttgtcttggaccctcttggtgacgtctatgcggcctggaaagccgtactcgtcttaggacaagcgggtcctcttttggttcaaggagtggtcctctgaccggccgtggaagtggacctgggactcgctgtcgtcgccgcacctcttgggaccgccgatgacggagtggttcacctactaggaccggcggctcgacttcacgaagccgttgtggcggcaccggttcacgttgcacttggtgctgctcctcaagacgctgtacgactctgagtagctgatgttgttccggcgggactcgttcaagttcgtcctgcacctctcgcgggacgtgcacaagttctggtggcacttgtcggagtagtcgctggtcgacgagtactctttggtggactctctggagtacccgcacgggatgacgttgatgtcgttcaagaccatagacctcgtgcggttctggccgctctggtcgcacgggttcacgaccgaccactggttaccgtcgatggacttgctctgggtgaagtcgctggtctagctcgtccttcggctgttgtactagtggctctacgactccttcctgatgtagttctctgtcccgtcgtggggggaccgggagtacctagacgagtacaagtcgtggtcgcggatggagtagtcgtagaaggacgtggaccacttctaggggtgggtgtctgtgtagttcccgccgtcgacggggttcggggtgtctgagtggttgttcccgtagacgtcgacgccgcggaagttccacgggccgcacttttggtagaccttctcctctattcgccggcgatgctggagctgatactttttttgattgtctataggagctgcggtggtacccgaccaggacgtagtaagacaaagaccaccggtgtcggtggccacaggtatcaagactaccgccgcgagtcctgacgacggacttcatgagagtctctttctaggggcggttccagcacgcctcgatgtctttcgttctcggaagagacccgacgagatagggacggtaggacaaagacggggccttctctagtgtccggcttgacacgcggctaggatttcttgacacccacgtcgtcgactacgtcgtagacctgttctggggtagaggagtcttcggacgagttccgactactaaaaatatgatcggtctaagaagtacaaacctggtttagttgaacactatggtacgagtttctccggagttaatataaactcaaaaattaaaaatactttttttgattgtcgttagtaccttcaggtgctaaaactctggctgctcaagttactaaagttacttctactgatacggtgttctcttaaggacttagggctactcgcgtactgcatgaacttagtacgactaatgttggacttaagaggagattaatcactactataactgttaaattagtcctttaagttaagagaaggttaagggagctacaccctatcattcttgaccctacctcaagaactctacaattgcagtacagttcggttagggtagggttgtagagtctacgtatttacctacccttcaaccaattacagactattagtactacggtcagttcccatatcaaaaaatgtacttcacctgtttctccgtctttattgtaaactgcaccacctctggaagtaggcgccgaccccgttgtttggttaacttatgtagtttttcctttctacctgactgagtaagttttaagagcgaataaacacagttttcaaaaacctgaatgtgttcaactgtaattagaatttacgacagagactccaccttaacgagttgaaccgctcctgaaagtttccgtttcagtcttcttcaagagtaccttgcttgtatacgtcctaatcccaagggtcgaacccaggatgaaaataaagtcttcctacccgaatgaagttctttgaactataagattacctggctttgaaagacaattaccagtttctacactaatatccctcctacgtttgccacgataggtaccatacatcttatctgttggacaagagtctcgttctgtagaagagggaagatttatagatgtcttaacctctattttaacacctctccgtccctttaaaaagaatactgaactaattttaccaccttggctatacgttgaacttcgactactttaatcgttctcttagttccggaaatcagggtgttaagggagtaaaacttttagtatagttctgaagacaactacttccccgtttttaactggctccatattctaaggaggtactagtctattactcacacttttgtcacctagagtgtgaccactaaatacctagcaagtctgtaaccccagtaggaaaatatctaataatgtgacctgatctttttaatgtaagggttcattggtacttctttctataactacacagtatacgttttcgtgaacgttcactaaatcgagcctaacaagataaagttgtcaagttactagtatttttcaccaagcacttacctctgaacgagggagtactagtagggaaattttcagtacaatttcttttatgtaccgggtgtcgacgagttcaagttctaaaacctctatttaccgtacttgaaggcgactaatttacaaaactttatgggctgaatgatctgggtagctattatatgagactgttttcagtaagttacttatccagtctccacaactttgtacaggcttacttaggcttgtgaggatagggatcatttttccacaacgtctgatacaacctgtgtttccgatggttaacctttcttaaagaatttctctaactactcttcccgaatctactactactagattaataaccagaatttcctttcctctcccttgacttcaaccgtccatctaaaaagagggattacagaacctttaacgctcttatgaaacattaatggcttataaactatttctgagtaaagcagggatacaaatttccggactgttaccgcctgctagattgacgtcagtaatttttctacaatctaaggagtaggccggttcctaacttcagtatactccgttaaacgtatcggttagtgtaactaatgctttttaccttattggtggtttccttcaatagtttgccgggtcacaaggctcaatacccggtcaagaatccaataggtaggaattagctctcttgagtacttaaaaaactcttttcagaatatatgatgttaccttctggtctgaactacgcacaagtgttgttgtgtgactagttaagttggagggttgctcaaacaaccgttcctgttctcccacctgaccttccagatgccgtttttcctacctcataggagttagatgaccaataagtttctctccgattttagtctttgtgacgacagtttcagaaccgtgttccactattagttcaataaacgtgtgtcatattttgcttctttagctctttgcaacatcttaatgtcccacgagagttagtttaccaaagattattactcttttaatactgacgttagttttatccctgtcccttcaatcctgaaaactatttactgctactctgatacgttagacgtctaatgaacttaatacctttttatggctaaaaggcacctcactaatctcccaatctctggttctctaccagtgctcactgaacacagtggttactggtttatgggtgaacacgattatattactcgagtcaaaggtgtttacgagagtggcatcgagtaaaacgactcttgggttagttacggtactatgtcatgttaataaaaccctgtaaacgatctgagaacaactactacgtactaggacgagaagcagttagtaacatacttcaagttctattctatggcccgaacgtgtcaagatgaaagtttatgcggtacaacataaacctgggaaggtaacctcctcacagcccgtacagaaacaggtccaaaaactaatctcggaagggtctagggcattgtctttcagagagtaagacctctaagtaggtacatgtacgagcttcactcgtagacttcctctactcacgtcataaacctttggggctctatcggttcaaagcttattgagtgtatctgttcgatcatcttctaggttggagagacttgtagcgatacccttactcaggtcgcttgaacaatttctgactccaattttttacgaattagcttagttctgtttggtagtccttggtccactaattcctacgttggtatataaacatagtacttctcctagccgagtcttcaaagaataccagttatttaggagacaagggatctaaaaattcacttaagtttagtccgtgaaaaaaccctcagcgtctgcccgagtagtcagataaagttttaagagcatgataagccttgaggaaattctttttcatagtatcccttaacctactaaactaacactcctcactccataggagaaactgtgtaaatccctttgaagtaaactcttcccctagtacattttacacctgtacaagtcgatgagtacgactgtgtaattctatgtttaggaccccggcatgtcaataaccctgttgacatggggtaggtaatctttacaacccaggtgttgtagcttttctctgaggaacacgtggtacattgtgtagtcccaagttaatacaaagacacgtaacaggtctgccctaggtactgcagaaatcaagtgcccctggtaacggacgaatagatcccagattttgtagacttagatgtagataaaacgtcggaaccctttccctttcgtttcagggtgactaattttctcgatgtgcagaatctctacgatagagaaccaaacaacttgggctgagatttgatcgttactgatatgaaagattgtaggtgagaaattgtccgcttcttacctggttttccgtcgtacccaagttttcttgtcccagacgggaagtatccaaaagctgtagagcctactcggtaccacccaagcgtagagtctcgtgacgtcgtaactggtccaactaccgttgatgtctgtggtactccctagaccctctagtcttaaagctgaaaaataaggttcgttgcaacgagatacgagtttaatggtggtgacaacgttctctgcctacctagtggtcaacatgtctagtaatagtataacggacattcaggacaaactctgggtatcttctctagtgggacctgagttcatacctgatgtgcgggggtctacatagggtacacgacttctgtacctccttaccccttccaagcacccctgttctctattttgtctagataggaaatcttcccttaaccttcttaaatcgtggacgactcgttaggatagttcagccgtctacatatccaaaagatatacctctgaaccgcatatcttttagatgagtacggctcctgtcaagagataaaggagatagatatgttccagcataatctccagctccaaagaattttcccaacgatctgcctaattactctcgttcaacgacggttcattatgtggcctcttcagaccgagtaaacttctccggccggttgcgtcacatgcctccaaactaaatgaactaactatttaactcacatagtggaggtaaggaaagagaatgatctagtcctggataatctctgcttaatctttgctaaggggtgttctagggttggaggataggctgttcgttggcactatacccccactaacagtctttaatgaagtttatggttacggcagattaacttttcccttttatgtctagtgtaataagtgttaataccaataagagtctacagaataggtatctgaagtaacctggtaagagataaaggtggtgggagaacgtttaggatatgttcggtaaaaatagaccctttctattcttactcaactctctcgaccgtttagaaagaagtaacgattctagtcctctccccacccttctgtatgtacactttaagaagtggttcctgtataataacacaggtctcctttagtctgtacgaacgttcaagccctaacgattcctattattatttctgtactcgatagggggaaccccttcccttaggtctccctgttaatgttgttagggacaaataatatgctggtggggaatgggtttctacgatctctacggaggttcttaggttttaggggacgacaggccttagtccaacccggttaatggttgaccgcgagtaatattttaagcctcatataatgtaccttacccttaggtaatgtccctgaagaactcaacacctctgccgaggcctccctactgacgacgtaatgatgctcttttacacgtatcgtctccttataagttatcagacaatcttaatagtcccagtcagtacgctccgcggagaggactcggggggtcacgggatctttgaaatcctcctctatttagctctacacatttaccactttgtacaacccttataggtagactgaatacactgggttcctgaaccctgataaaggaggctgagtttcgtccgaaccccgaagtttaactaaattaacattacctataccttcaagccctaagaagatgatcggacttttaactctgcttacaatctttaatacacgtggcctaaaacctactcgttcctcaaaattagatgttctgaataccttgtatataaacactctcgcttttcttacgtcattgttaggaaccagggtacaagttctgccagctgaatcaagtttgtcttaaatcatcaagagtttgcagacttcatatataccatacatttccaaacttctttaattagctacttgggttagggctaaccagaaggtagttacttaggacctttttggacatgcgtaaggtcagtagtcttgtccttaaacggtctcgtttcttccaatcatgtatgaaatggaactgtccataagggagggttaagtaaggactaggaaaacatttgtaactctgatacgatgtttataagcctcatgggtgcccacacagagtacgccgacggaattttagtagactatctggacgtctaaataactggtaatcggaaaaaatataccgctaatatagcataatattgtagttagtatagtctcatcctggctatggaggcttggggggtagtctaccttaacgtgttttacacccctagcgatattgaccatattcgaaaaccgactcaaactacctctttctgtaaggtgatatagttgtcacaaatcgtcaataggtcgttagtaagggctaatccaccctccgacaaagtcattttcctcctatgttcgtcttcacctcatgatctccactacccgagggttttctatgggcttaaagtctgaggaaccggggttagcccttgacctagtctagagaccttaaccaggctttggttcaagcagatttaggtaagttactctagaacaagttagtcgatacagcatgtcacctattagtaaactttaccagtttaaacgcttctttgtgtccttactaacttacctagttatctgcttaaagttttcttctggccagatatgactacaacttctcactggatgtgctccttttgagaacctctctaattttttagtactcctctgaggtttgaaattcatactttttttgaaactaggaattctgggagaacaccaaaaataaaaaatagaccaaaacaccagaagcatcggacgagttccgact

SEQ ID NO: 8 - RNA, 12603 nt, Vesicular stomatitis virus
  source 1..12603: mol_type = genomic RNA
tgcttctgtttgtttggtaataatagtaattttccgagtcctctttgaaattgtcattagttttacagacaatgtcagttctcttagtaactgttgtgtcagcatcaaggttttgaaggacgtttactcctaggtcaccttatgggccgtctaatgaagtcttttagtttcctctaaggagaaatgtagttatgatgtttttcaaacagtctagattctcctatacagatggttccggagtttaggcctttacatagttagtatgtacagttgtcgatgaacatacctcgtaatttcctgtaggccccattcaacctatttctaaccagttcaaagccttatttgtagccctttcgtcccctatgttagccttataaactggaacataggaactttcgggacctgccgcatgaaggtctacctcatagcctacgaaggtcttggtcgcgtctactgtttaccaacggaaacatagatgaaccgaatatgtctcacccgtcttgtgtttacggacttatgtcttttttcgagtacctacccgactgtttagttacgttttactagttacttgtcaaacttggagaacacggtcttccagcactgtaaaaactacacacccctttactgtcattaatgtgtttttaacagcgacgtcacctgtacaagaaggtgtacaagttttttgtacttacacggagcaagtctatgccttgataacaaaggtctaagtttctaacacgacgtaaccgttgtaaacctgtggagacgttttattggccttacagatgtcttctacattgctggacctagaacttggctcttcaacgtctactttaccaggtttactacgaaggtccggttctttaactgttccggctaagtatgtacggaataaactagctgaaacctaacagaagattcagaggtataagaaggcagtttttgggacggaaggtgaagacccccgttaactgtcgagaagacgagtctaggtggtctcgttccttacgggctgtcggactactgtaactcatatgtagagaatgatgtcgtccaaacaacatgcgaatacgtcatcctaggagacggctgaaccgtgttgtcaaaacacaacctctattgtttatgtgaggtctactatcatggcctcctaactgctgattacgtggcggtgttccgtctctacaccagcttaccgagcctaccaaacttctagttttgtcttttggctgaggactatactacgtcatacgcttttctcgtcagtacagtgacgttccggattctctcttctgttaaccgttcatacgattcagtcttaaactgtttactgggatattaagagtctagtggataatatataatacgatgtatactttttttgattgtctatagtacctattagagtgttttcaagcactcatagagttcaggataagagcagacctagtccgccatcctctctatctactctagcttcgtgttgctcgacttttcaggttaatactcaacaaggttctcctacctcaccttctcgtatgattcgggagaataaaagtccgtcgtctactaagactgtgtcttagacttggtctttaacttctgttagttccgaacatacgtggtctaggtcttcgactcgttcaacttccgaaatatgtccccggaaatctactgatacgtctactccttcacctacaacataaatgaagcctgacctttgtcggactcgaacttagactgctcgtacctttctggaatgccaactgtagcggtctcccaaattcacctctcgtctttagggtcaccgaaagctgctaatttcgtcagcacgtttcacggtttatgaccttagaccgtctcacgtgtaaacttcgtagccctcttccccagtaatacttcctcgcggtctattgaggcctacatatattccagtgaggtcactacttgtgtgtaggcagggttagtcttcgtcatagtctacaaaccagagagagtttctgtaggtactgaaaggttgggttctttcgttcagaagtcggagagtggtataggaacctacttaacaagagtagatctcctctcaagtagagacagcctccactgcctgcttacagagtatttctccggtaggacgagccggactctatgtttttcaacatgttagtccgctctcagtttataagagacatctgatactttttttcattgtctatagtgctagattcacaatagggttaggtaagtagtactcaaggaatttcttctaagagccagacttcccctttccattctttagattctttaatccctagcgtggtgggggaatacttctcctgtgatcgtacctcatacgaggctcgcgaggttaactgtttaggataaaacctcaactgctctacctgtggatactaggcttagttaattctatactctttaagaagaaatgtcacttttactgccaatctagattagcaggcaagtcttgtatgagtctacaccgtcggcgacatagggtaaccctagtgtacatgtagccttaccgtccctttgcagggaagatgttttagaaccgaaaaaacccaagaagattagatttccggtgaggtcgccataaccgtctagttccagttggtctcatagtgcgagtgacgcttccgtcccgaataaacggtgtatcctaccccttctggggagggtacgagttacatggtctcgtgaagtcttctggtaagttatatccagaaatgttcccttgctaactcgagtgttactggtagatgctactactcagtgaccttcgtcgaggatactagaccctagtaaagttaagaaggtttaaaagactaaagtctctcttccggaattacaaaccggactaacagctctttttccgtagacctcgcacccaggacctgagatagccggtgaagtttactcgatcagattgaagatcgaagacttgttaggggccaaatgagtcagaggggattaaggtcggagagcttgttgattataggacagaaaagatagggatactttttttgattgtctctagctagacaaatgcgcagtgcctagggggcccgacgtccttaagcggtggtacccggtctagcactggtacaagctccgggacggggtgtagtagctgctccactagttgtagcactagtagcacgagtagtagtagtggtcgtagttccggcacatgttgaagcggtggacgccgtaggaccgggaccactcgaaggacaaggaccggccgtcttcgacgccgtacatgccggacttaccggggctatagatgttcccgcacatggtcaagttctcgcacctcaagctgtactcggtggacttggagtggtacgggttgcggacgtcgcggttgttatcggtggtgatgtagtcgtacccgtcgtcgccggacctcaactggaagtggttgctgtcgtaggacttggtgttgaagacgttggagtggtcgcggaagttgttcttttggaagctggtgtgggagtactcgtagcactcgtcggacgtggactcgtagtctccgttgtcgttggtgttccggcactcgacgctgaagttgttgccgtagtggtaggtcatgttggactcgaagtcgctaggagtctcgcggtagtcggtcacgtcttggaagtctccgtctcacgacctgtacaagtcttggcggaagccgccgttcatgtactcttcgccgaccccgacccggccgtcgctgccgttctggtggaccacgtcggtctggtcgatggtcatggagtagtaggtcttgtcttggaccctcttggtgacgtctatgcggcctggaaagccgtactcgtcttaggacaagcgggtcctcttttggttcaaggagtggtcctctgaccggccgtggaagtggacctgggactcgctgtcgtcgccgcacctcttgggaccgccgatgacggagtggttcacctactaggaccggcggctcgacttcacgaagccgttgtggcggcaccggttcacgttgcacttggtgctgctcctcaagacgctgtacgactctgagtagctgatgttgttccggcgggactcgttcaagttcgtcctgcacctctcgcgggacgtgcacaagttctggtggcacttgtcggagtagtcgctggtcgacgagtactctttggtggactctctggagtacccgcacgggatgacgttgatgtcgttcaagaccatagacctcgtgcggttctggccgctctggtcgcacgggttcacgaccgaccactggttaccgtcgatggacttgctctgggtgaagtcgctggtctagctcgtccttcggctgttgtactagtggctctacgactccttcctgatgtagttctctgtcccgtcgtggggggaccgggagtacctagacgagtacaagtcgtggtcgcggatggagtagtcgtagaaggacgtggaccacttctaggggtgggtgtctgtgtagttcccgccgtcgacggggttcggggtgtctgagtggttgttcccgtagacgtcgacgccgcggaagttccacgggccgcacttttggtagaccttctcctctattcgccggcgatgctggagctgatactttttttgattgtctataggagctgcggtggtacccggtgtgttcttccgtcccttgttcggggtcgttcacggggatggacttgaagaaagtcgacgaccacgaccggccggactcggtgaaaacaagaccgcactaggtgcactggtttcttcactttctccagcggtgtgactcgacgccggtgttgcaaagtcaccttcttgaccgggtctggtcttagatgaccgtctttcttttcttttaccacgactggtactactcgccgctgtacttgtagaccgggctcatgttcttggcctggtagaagctgtagtggttgttggactcgtagcactaagaccgggactccggaagactactcccgtggatactcacgcaccacgacttcatgctcttcctgcggaagttcgcgctcgtagaccggcttcactgtgactcgcacttccggctgaaagggtgtggatcgtagtcgctgaagctctaggggtggtcgttgtagtctgcctagtagacatcgtggtcgccgccgaaaggtctcggagtagacagaaccgaccttttgccgctccttgacttgcggtagttgtggtggcacagagtcctggggctctgtctcgacatacggcacaggtcgttcgacctgaagttgtactggtggttggtgtcgaagtacacggactaattcatgccggtggactctcacttggtctggaagttgaccttgtggtggttcgttctcgtgaaggggctgctgttctgggtgtggacaggaggtacaggacgaggtcttgacgagccgcctgggaggcacaaagacaagggaggtttcggattcctgtgggactactagtcgtcttggggacttcactggacgcaccaccacctacacagagtgctcctgggtcttcacttcaagttaaccatgcacctgccgcaccttcacgtgttgcggttctggttcggatctctccttgtcatgttgtcgtggatgtctcaccacaggcacgactggcacgacgtagtcctgaccgacttgccgtttctcatgttcacgttccacaggttgttccgggacggacgaggatagctcttttggtagtcgttccggttccctgtcggggcccttggagttcacatatgggacggaggatcggcccttctctactggttcttagtccacagggactggacggagcacttcccgaagatgggaaggctatagcggcaccttaccctctcgttaccggtcggactcttgttgatgttctgttggggaggacacgacctgtcgctgccgagtaagaaggacatgtcgtttgactggcacctgttctcgtctaccgtcgtcccgttacacaagtcgacgtcgcactacgtgctccgggacgtgttggtgatgtgggtcttttcggactcggacagaggaccgactagttgatcggtctaagaagtacaaacctggtttagttgaacactatggtacgagtttctccggagttaatataaactcaaaaattaaaaatactttttttgattgtcgttagtaccttcaggtgctaaaactctggctgctcaagttactaaagttacttctactgatacggtgttctcttaaggacttagggctactcgcgtactgcatgaacttagtacgactaatgttggacttaagaggagattaatcactactataactgttaaattagtcctttaagttaagagaaggttaagggagctacaccctatcattcttgaccctacctcaagaactctacaattgcagtacagttcggttagggtagggttgtagagtctacgtatttacctacccttcaaccaattacagactattagtactacggtcagttcccatatcaaaaaatgtacttcacctgtttctccgtctttattgtaaactgcaccacctctggaagtaggcgccgaccccgttgtttggttaacttatgtagtttttcctttctacctgactgagtaagttttaagagcgaataaacacagttttcaaaaacctgaatgtgttcaactgtaattagaatttacgacagagactccaccttaacgagttgaaccgctcctgaaagtttccgtttcagtcttcttcaagagtaccttgcttgtatacgtcctaatcccaagggtcgaacccaggatgaaaataaagtcttcctacccgaatgaagttctttgaactataagattacctggctttgaaagacaattaccagtttctacactaatatccctcctacgtttgccacgataggtaccatacatcttatctgttggacaagagtctcgttctgtagaagagggaagatttatagatgtcttaacctctattttaacacctctccgtccctttaaaaagaatactgaactaattttaccaccttggctatacgttgaacttcgactactttaatcgttctcttagttccggaaatcagggtgttaagggagtaaaacttttagtatagttctgaagacaactacttccccgtttttaactggctccatattctaaggaggtactagtctattactcacacttttgtcacctagagtgtgaccactaaatacctagcaagtctgtaaccccagtaggaaaatatctaataatgtgacctgatctttttaatgtaagggttcattggtacttctttctataactacacagtatacgttttcgtgaacgttcactaaatcgagcctaacaagataaagttgtcaagttactagtatttttcaccaagcacttacctctgaacgagggagtactagtagggaaattttcagtacaatttcttttatgtaccgggtgtcgacgagttcaagttctaaaacctctatttaccgtacttgaaggcgactaatttacaaaactttatgggctgaatgatctgggtagctattatatgagactgttttcagtaagttacttatccagtctccacaactttgtacaggcttacttaggcttgtgaggatagggatcatttttccacaacgtctgatacaacctgtgtttccgatggttaacctttcttaaagaatttctctaactactcttcccgaatctactactactagattaataaccagaatttcctttcctctcccttgacttcaaccgtccatctaaaaagagggattacagaacctttaacgctcttatgaaacattaatggcttataaactatttctgagtaaagcagggatacaaatttccggactgttaccgcctgctagattgacgtcagtaatttttctacaatctaaggagtaggccggttcctaacttcagtatactccgttaaacgtatcggttagtgtaactaatgctttttaccttattggtggtttccttcaatagtttgccgggtcacaaggctcaatacccggtcaagaatccaataggtaggaattagctctcttgagtacttaaaaaactcttttcagaatatatgatgttaccttctggtctgaactacgcacaagtgttgttgtgtgactagttaagttggagggttgctcaaacaaccgttcctgttctcccacctgaccttccagatgccgtttttcctacctcataggagttagatgaccaataagtttctctccgattttagtctttgtgacgacagtttcagaaccgtgttccactattagttcaataaacgtgtgtcatattttgcttctttagctctttgcaacatcttaatgtcccacgagagttagtttaccaaagattattactcttttaatactgacgttagttttatccctgtcccttcaatcctgaaaactatttactgctactctgatacgttagacgtctaatgaacttaatacctttttatggctaaaaggcacctcactaatctcccaatctctggttctctaccagtgctcactgaacacagtggttactggtttatgggtgaacacgattatattactcgagtcaaaggtgtttacgagagtggcatcgagtaaaacgactcttgggttagttacggtactatgtcatgttaataaaaccctgtaaacgatctgagaacaactactacgtactaggacgagaagcagttagtaacatacttcaagttctattctatggcccgaacgtgtcaagatgaaagtttatgcggtacaacataaacctgggaaggtaacctcctcacagcccgtacagaaacaggtccaaaaactaatctcggaagggtctagggcattgtctttcagagagtaagacctctaagtaggtacatgtacgagcttcactcgtagacttcctctactcacgtcataaacctttggggctctatcggttcaaagcttattgagtgtatctgttcgatcatcttctaggttggagagacttgtagcgatacccttactcaggtcgcttgaacaatttctgactccaattttttacgaattagcttagttctgtttggtagtccttggtccactaattcctacgttggtatataaacatagtacttctcctagccgagtcttcaaagaataccagttatttaggagacaagggatctaaaaattcacttaagtttagtccgtgaaaaaaccctcagcgtctgcccgagtagtcagataaagttttaagagcatgataagccttgaggaaattctttttcatagtatcccttaacctactaaactaacactcctcactccataggagaaactgtgtaaatccctttgaagtaaactcttcccctagtacattttacacctgtacaagtcgatgagtacgactgtgtaattctatgtttaggaccccggcatgtcaataaccctgttgacatggggtaggtaatctttacaacccaggtgttgtagcttttctctgaggaacacgtggtacattgtgtagtcccaagttaatacaaagacacgtaacaggtctgccctaggtactgcagaaatcaagtgcccctggtaacggacgaatagatcccagattttgtagacttagatgtagataaaacgtcggaaccctttccctttcgtttcagggtgactaattttctcgatgtgcagaatctctacgatagagaaccaaacaacttgggctgagatttgatcgttactgatatgaaagattgtaggtgagaaattgtccgcttcttacctggttttccgtcgtacccaagttttcttgtcccagacgggaagtatccaaaagctgtagagcctactcggtaccacccaagcgtagagtctcgtgacgtcgtaactggtccaactaccgttgatgtctgtggtactccctagaccctctagtcttaaagctgaaaaataaggttcgttgcaacgagatacgagtttaatggtggtgacaacgttctctgcctacctagtggtcaacatgtctagtaatagtataacggacattcaggacaaactctgggtatcttctctagtgggacctgagttcatacctgatgtgcgggggtctacatagggtacacgacttctgtacctccttaccccttccaagcacccctgttctctattttgtctagataggaaatcttcccttaaccttcttaaatcgtggacgactcgttaggatagttcagccgtctacatatccaaaagatatacctctgaaccgcatatcttttagatgagtacggctcctgtcaagagataaaggagatagatatgttccagcataatctccagctccaaagaattttcccaacgatctgcctaattactctcgttcaacgacggttcattatgtggcctcttcagaccgagtaaacttctccggccggttgcgtcacatgcctccaaactaaatgaactaactatttaactcacatagtggaggtaaggaaagagaatgatctagtcctggataatctctgcttaatctttgctaaggggtgttctagggttggaggataggctgttcgttggcactatacccccactaacagtctttaatgaagtttatggttacggcagattaacttttcccttttatgtctagtgtaataagtgttaataccaataagagtctacagaataggtatctgaagtaacctggtaagagataaaggtggtgggagaacgtttaggatatgttcggtaaaaatagaccctttctattcttactcaactctctcgaccgtttagaaagaagtaacgattctagtcctctccccacccttctgtatgtacactttaagaagtggttcctgtataataacacaggtctcctttagtctgtacgaacgttcaagccctaacgattcctattattatttctgtactcgatagggggaaccccttcccttaggtctccctgttaatgttgttagggacaaataatatgctggtggggaatgggtttctacgatctctacggaggttcttaggttttaggggacgacaggccttagtccaacccggttaatggttgaccgcgagtaatattttaagcctcatataatgtaccttacccttaggtaatgtccctgaagaactcaacacctctgccgaggcctccctactgacgacgtaatgatgctcttttacacgtatcgtctccttataagttatcagacaatcttaatagtcccagtcagtacgctccgcggagaggactcggggggtcacgggatctttgaaatcctcctctatttagctctacacatttaccactttgtacaacccttataggtagactgaatacactgggttcctgaaccctgataaaggaggctgagtttcgtccgaaccccgaagtttaactaaattaacattacctataccttcaagccctaagaagatgatcggacttttaactctgcttacaatctttaatacacgtggcctaaaacctactcgttcctcaaaattagatgttctgaataccttgtatataaacactctcgcttttcttacgtcattgttaggaaccagggtacaagttctgccagctgaatcaagtttgtcttaaatcatcaagagtttgcagacttcatatataccatacatttccaaacttctttaattagctacttgggttagggctaaccagaaggtagttacttaggacctttttggacatgcgtaaggtcagtagtcttgtccttaaacggtctcgtttcttccaatcatgtatgaaatggaactgtccataagggagggttaagtaaggactaggaaaacatttgtaactctgatacgatgtttataagcctcatgggtgcccacacagagtacgccgacggaattttagtagactatctggacgtctaaataactggtaatcggaaaaaatataccgctaatatagcataatattgtagttagtatagtctcatcctggctatggaggcttggggggtagtctaccttaacgtgttttacacccctagcgatattgaccatattcgaaaaccgactcaaactacctctttctgtaaggtgatatagttgtcacaaatcgtcaataggtcgttagtaagggctaatccaccctccgacaaagtcattttcctcctatgttcgtcttcacctcatgatctccactacccgagggttttctatgggcttaaagtctgaggaaccggggttagcccttgacctagtctagagaccttaaccaggctttggttcaagcagatttaggtaagttactctagaacaagttagtcgatacagcatgtcacctattagtaaactttaccagtttaaacgcttctttgtgtccttactaacttacctagttatctgcttaaagttttcttctggccagatatgactacaacttctcactggatgtgctccttttgagaacctctctaattttttagtactcctctgaggtttgaaattcatactttttttgaaactaggaattctgggagaacaccaaaaataaaaaatagaccaaaacaccagaagca